(19) United States
(12) Patent Application Publication
(10) Pub. No.: US 2015/0173898 A1
(43) Pub. Date: Jun. 25, 2015

(54) Transcatheter Mitral Valve Replacement Apparatus

(71) Applicants: William Joseph Drasler, Minnetonka, MN (US); Mark Lynn Drasler, Greenfield, MN (US); Richard Charles Kravik, Champlin, MN (US); William Joseph Drasler, II, Minnetonka, MN (US)

(72) Inventors: William Joseph Drasler, Minnetonka, MN (US); Mark Lynn Drasler, Greenfield, MN (US); Richard Charles Kravik, Champlin, MN (US); William Joseph Drasler, II, Minnetonka, MN (US)

(21) Appl. No.: 14/559,912

(22) Filed: Dec. 3, 2014

Related U.S. Application Data
(60) Provisional application No. 61/963,412, filed on Dec. 3, 2013.
(60) Provisional application No. 61/955,293, filed on Mar. 19, 2014.
(60) Provisional application No. 61/995,283, filed on Apr. 7, 2014.
(60) Provisional application No. 61/998,131, filed on Jun. 19, 2014.
(60) Provisional application No. 61/999,333, filed on Jul. 23, 2014.

(51) Int. Cl. A61F 2/24

(52) U.S. Cl. CPC A61F 2/2445; A61F 2210/0057; A61F 2230/0069

(57) ABSTRACT

Transcatheter mitral valve replacement is provided which includes a stabilizing stent portion for placement at a mitral annulus, with a narrowed waist portion to avoid significantly dilating the annulus, and expanded bulb portions at the atrial and ventricular ends to secure the device in position. A housing is attached to the stabilizing stent and reduces in diameter to a smaller tract portion where replacement valve leaflets are secured. The resulting smaller replacement mitral valve offers benefits including a lower introduction profile, yet fits and secures to the native annulus by the securing stent and housing. Expansion limiting elements can limit expansion of portions of self-expanding stent if desired, avoiding excessive mitral annulus dilation or providing a fixed diameter in other portions of the stent or housing. Two-step implantation can be accomplished by making the housing functional as a temporary valve.

### RELATED PATENT DOCUMENTS

This patent application claims benefit from the earlier filed U.S. Provisional Applications No. 61/963,412 filed Dec. 3, 2013 entitled “Small Diameter Mitral Valve”, No. 61/955,293 filed Apr. 7, 2014 entitled “Adapter for Small Diameter Mitral Valve”, No. 61/995,283 filed Apr. 7, 2014 entitled “Post Dilation Stent”, No. 61/998,131 filed Jun. 19, 2014 entitled “Mitral Valve Stent Design”, No. 61/999,333 filed Jul. 23, 2014 entitled “Mitral Valve Expandable Housing”, all pending, which are hereby incorporated into this application by reference as if fully set forth herein.

### BACKGROUND

1. Field of the Invention

The present invention pertains generally to medical devices, and particularly to medical devices for catheter-based treatments, and more particularly, for transcatheter replacement of the atrioventricular valves of the heart.

2. Description of the Prior Art

Valves of the heart including the aortic valve and mitral valve can become hardened from atherosclerotic plaque and calcium and no longer function normally. Alternately these valve can prolapse and allow blood to pass through the valve in a retrograde manner that is opposite to the normal direction of flow through the valve. Such regurgitant flow can require repair or replacement of the valve. Surgical repair or replacement of such valve is often the gold standard at present for those patients able to withstand the rigors of surgery. An alternate and less invasive approach would be desirable via access to the valve from the femoral vasculature, vasculature of the arms, the apex of the heart, aortic access, or via other less invasive sites.

Transcatheter aortic valve replacement (TAVR) has evolved to become an accepted less invasive procedure for replacing diseased or incompetent aortic valves in high risk patients. Such less invasive surgical procedures are not as well developed for replacing abnormally functioning mitral valves.

Often the regurgitant mitral valve is a result of excessive expansion of the left ventricle (LV) leading to abnormal tension and angulation imposed on the mitral valve leaflet. The mitral valve leaflet is often unable to coapt properly with its neighboring leaflet and will therein allow retrograde blood flow to occur through the valve. The mitral valve annulus can also expand in diameter reducing the ability of the mitral valve leaflets to coapt properly. It is therefore not desirable to place a stent into the mitral annulus to push it further outwards as is done with TAVR procedures onto the aortic valve annulus.

The mitral anatomy also provides that the anterior mitral leaflet not only helps close the mitral annulus during systole, but also provides one surface of the left ventricular outflow tract (LVOT) during systolic pumping of blood out of the LV. It is therefore not acceptable to expand a stent indiscriminately outwards as is done in TAVR due to the potential for blockage of the LVOT by the anterior mitral valve leaflet.

An approach for replacement of dysfunctional mitral valves via less invasive means is needed that will not apply excessive outward force to the mitral annulus, yet still be able to secure the replacement mitral valve without migration. The device should also not cause interference with the anterior mitral valve leaflet in a way that could affect blood outflow through the LVOT.

Self-expanding (SE) stents are used to provide support to tubular vessels of the body, for delivery of drugs to these vessels and to house structures such as valves that can be delivered via transcatheter methods to the site of a lesion. One application of a self-expanding stent is in providing a housing for a stent-valve used for implantation of a transcatheter aortic valve replacement (TAVR) device. Other applications for self-expanding stents or stent valves include the treatment of coronary arteries, arteries or veins of the lower body or leg, the esophagus, tubular ducts, or other tubular vessels of the body. Although this discussion will focus on the use of SE stents or stent-valves used in the treatment of stenotic aortic valve disease, it is understood that the discussion applies to stents and stent-valves used to treat other cardiac valves, other tubular tissues, and other vessels or chambers of the body that can be treated by a SE stented device or that are currently treated by a balloon-expandable (BE) stent and could alternatively be treated by a SE stent or stented device.

SE stents tend to have a force profile vs their diameter that exposes the tubular tissue of the body to an ongoing outward force after it has been allowed to expand from its small diameter as constrained within a delivery sheath for delivery to the lesion site to its larger diameter configuration to make contact with the vessel wall. For a transcatheter aortic valve replacement (TAVR) device, or for a transcatheter mitral valve replacement (TMVR) device, for example, the SE stent could contain a replacement tissue valve that is attached to the inner lumen of the stent. After the TAVR stent, for example, is located adjacent the aortic annulus, it is released from the constraining sheath and allowed to expand into contact with the walls of the aortic annulus, the left ventricular outflow tract (LVOT) and also may contact the aorta for some specific designs. The SE stent can continue to impose an outward force against the wall of the tissues in which it is in contact. For a SE stent-valve, the outward force must be large enough the ensure that a good seal is made with the tissues to ensure that perivalvular leaks are not present and to ensure that stent migration either upstream or downstream does not occur. Excessive outward force against the tissue of the annulus or vessel can cause the struts of the stent to migrate into or through the annulus or vessel wall or can apply a continual outward force causing trauma to the tissue, leakage from the vessel wall, thrombosis, infection, physiological dysfunction, and possibly death. It is difficult to provide a SE stent that makes contact with the vessel wall but does not provide too large of an outward force against the wall. For a TMVR stent, the continued outward force against the mitral annulus that has been already dilated due to functional mitral disease can similarly produce undesirable conditions stemming from further annular dilation.

For the case of treatment of aortic stenosis with a SE aortic stent-valve or SE TAVR device, the application of a continued outward force is generated by some TAVR devices as the stent makes contact with the LVOT near the membranous septum where the nerves that carry the electrical stimulating signals to the heart cross over from the right atrium into the inner surface of the LV. The nerve in this region can include, for example, the Bundle of His, and carries electrical stimulating signals to cause contraction of the LV. The continued exposure of such nerves can result in left bundle branch block (LBBB) and can result in the need to place a permanent pacer into this patient. For the case of TMVR, the continued force against the mitral annulus can cause further dilation of the mitral annulus resulting in potential centro-valvular leaks or poor valvular function, such as mitral regurgitation.

The SE stent-valve has an advantage over some BE stent-valves used for TAVR or TMVR due to the lower profile for the SE device and also the ability to achieve a functioning valve during the deployment or release of the valve from the constraining sheath. The LVOT, atrioventricular blood flow path, or other blood flow path is not temporarily blocked as it is for delivery of a BE stent-valve or BE TAVR device which blocks blood flow while a balloon is used to expand the BE TAVR device into position adjacent the native valve leaflets. There is continued need for a SE stent valve that provides low profile and does not block the flow of blood during its delivery, while avoiding the continued outward force that can result in the need for a permanent pacemaker or creation of trauma to the tubular vessel of the body, blood leakage, or poor valvular function.

Mitral valvular disease can be of a primary or congenital origin resulting in mitral regurgitation due to mitral valvular prolapse. In this instance the mitral annulus can be of a normal diameter and application of a SE stent against the mitral annulus to hold a TMVR device in place without migration or perivalvular leakage may be acceptable. For functional mitral disease stemming from a left ventricular enlargement, the mitral annulus is often dilated resulting in mitral regurgitation. In this case application of a continued outward force against the mitral annulus is undesirable.

There is continued need for improved devices and methods for less invasive mitral valve replacement, with lower profile, with reduced complications, and minimizing or eliminating the need to block blood flow during deployment, without causing ongoing annular dilation, and without interfering with the LVOT, cardiac conduction pathways, or other structures and tissues.

### SUMMARY OF THE INVENTION

The general purpose of the present invention is to provide a better way of replacing a dysfunctional mitral valve.

According to embodiments of the present invention, there is provided apparatus for transcatheter placement of a replacement mitral valve.

According to embodiments of the present invention, there are provided methods for transcatheter placement of a replacement mitral valve.

According to embodiments of the present invention, there is provided apparatus for placement of a smaller transcatheter replacement valve into a larger valve annulus.

According to embodiments of the present invention, there are provided methods for placement of a smaller transcatheter replacement valve into a larger valve annulus.

According to embodiments of the present invention, there is provided self-expanding stent apparatus which limit expansion of at least a portion of the stent.

In one example, the present invention comprises a valve intended for transcatheter replacement of the aortic or mitral valve of the heart. Although most of the discussion is for its application as a mitral valve replacement through subclavian veins of the arms, femoral veins of the leg, or apex of the heart, it is understood that the invention is also applicable to aortic or mitral valve replacement via femoral artery access, aortic access, apical access, or other less invasive access.

The delivery systems used in current TAVR procedures are applicable in part to the delivery system used for delivery of the present invention as a mitral valve replacement. As a mitral valve replacement, generally access to the mitral valve is gained either via crossing the intra-atrial septum or via access to the LV from the apex of the heart.

Some embodiments of the present invention comprise a stent that is expanded via a mechanical means such as a balloon; other embodiments are formed from a self-expanding material and are released via withdrawal of a sheath in a manner similar to that taken with some current TAVR devices.

In one embodiment a valve that is able to be used for Transcatheter Mitral Valve Replacement (TMVR) has a BE stent with a varying-diameter stabilizing portion, with larger ends and a narrower central waist. This varying-diameter stabilizing portion surrounds the mitral annulus with an upper stent bulb pushing outwards against the wall of the left atrium (LA) above the annulus and a lower stent bulb pushing outwards against the mitral leaflets below the mitral annulus in the left ventricle, and a central waist that is expanded less than the upper and lower stent bulbs. Once this BE stent has been deployed, the central waist of the BE stent places no further outward force against the annulus that could tend to cause further annular dilation. The upper and lower stent bulbs prevent the valve from migrating either toward the LA or toward the LV.

The BE stabilizing portion (or SE in some embodiments) of the stent located upstream and attached to a downstream SE tract portion that forms the outflow tract from the LA to the LV. This tract portion is considerable smaller in diameter than the waist of the stabilizing portion; its purpose is to hold the native leaflets outwards and provide a space for function of the replacement leaflets which are attached to the tract portion while not impinging upon the native anterior mitral valve leaflet to cause a restriction in blood outflow through the LVOT, or impinge upon the lateral wall of the LV. The smaller diameter of the tract portion is about 2-2.5 cm, for example, and is significantly (at least 20% less) less than the diameter of the waist which is located adjacent the mitral annulus (typically 2.7-3.5 cm in diameter or more). The small diameter portion can range in some embodiments from 15-30% less than the diameter of the mitral annulus or the diameter of the waist of the stabilizing stent.

The smaller diameter tract portion ensures that the anterior leaflet of the mitral valve is not pushed outward to obstruct the LVOT. Even though the provided diameter for flow is less than that provided by a healthy mitral valve, it is adequate to sustain normal everyday function of most patients under most circumstances. Further, the smaller diameter for the tract portion provides for less valvular material required to span the smaller opening; hence, the profile of the device for delivery to the site will be less and its flexibility will be greater.

A covering is attached to the SE stent portion to prevent regurgitant flow through the TMVR; the covering can extend throughout the entire stent structure, including the BE stabilizing portion to ensure that retrograde blood leakage does not occur. The covering can be formed from a fabric material such as a woven or knitted fabric or a polymeric sheet material; the material for the fabric can be nondistendable fabric such as PET or Nylon that resists expansion of the covering upon exposure to expansion forces. Alternately, the covering can be formed in some embodiments from an expandable material such as polyurethane or spandex that will allow expansion of the covering; the restraining forces from the covering can serve to limit excessive expansion.

The leaflets contained within and attached to the tract portion (or for some embodiments the leaflets can be attached to the stabilizing stent) of the stent can be a bileaflet valve similar to that found in the venous system of the body or the native mitral valve, or can be a trileaflet valve similar to that found in a native aortic, pulmonary, or tricuspid valve of the heart. The material for the valve leaflets can be bovine, porcine, or other animal pericardium or other tissue, collagen, fibrin, or other valve material commonly used or anticipated for use in replacement valves. Alternately, a synthetic valve material can be used including material such as polyurethane, ePTFE, NiTi, carbon, or composite materials, or synthetic analogs of biological materials. Valve materials can also include portions or components comprising other metal or ceramic materials. Attachment of the leaflets to the tract portion (or stabilizing portion) of the stent follows a curved or crown-shaped path that is similar to that found in the attachment of aortic valve leaflets or venous valve leaflets to their respective conduit. Bileaflet replacement mitral valve leaflets that are oriented such that the major axis of the native mitral annulus is oriented with the commissures of the bileaflet valve will allow for improved coaptation of the leaflets over a greater range of ovality of the mitral annulus resulting in less regurgitation and improved durability for the replacement leaflets. Valves having four leaflets could also be used to replace dysfunctional mitral valves.

The BE stabilizing portion of the stent can be formed from various stent geometries by incorporating an expansion limitation element into the waist portion of the stent. A expansion limitation element is placed into the stent geometric structure that limits the amount of radial expansion that the waist portion can attain. The upper and lower bulb regions on each side of the waist are able to expand freely and extend in diameter further than the waist. The result is a cupped-shape stent that grasps above and below the mitral annulus to prevent migration, whereas the waist does not exert a force against the mitral annulus.

The BE stabilizing stent portion can be attached to the SE tract portion via a number of means. In one embodiment the BE stent and the SE stent are contiguous and formed from a metal such as NiTi. The BE portion is machined such that the expansion sites focus the expansion deformation and thereby undergo plastic deformation. This can be accomplished by forming expansion sites of a stent structure such that the junction where two struts meet is an expansion site having a width and thickness (radial dimension) which are smaller than those of the struts and making the length of this expansion site small in comparison to a strut length. Thus a single stent formed from NiTi can undergo BE character in the stabilizing portion as well as normal SE behavior in the tract portion. Alternately, the stent can be formed from stainless steel or one of the many alloys being currently used for stents. The stainless steel stent structure can be designed and machined to reduce the deformation focus by increasing the length of the expansion sites and thereby remain generally elastic in comparison to other portions of the stent. Thermal treatment of either the NiTi or the standard stainless steel or alloy can also alter the behavior of the stent to perform as a BE or SE stent. A soft stainless steel, for example, will behave plastically like a BE stent and a full hard metal will behave elastically like a SE stent. As an alternate embodiment, a standard BE stent can be attached to a SE stent via a number of attachment methods including welding, brazing, sewing, interweaving, forming loops, bonding, or other means.

The balloon used for delivery in this embodiment can be any balloon currently found in the industry for dilation of large vessels, tubular tissue spaces or openings. Such a balloon can be a cylindrical balloon when the waist of the stent is formed in a manner that restricts the diameter.

The delivery system for this embodiment could include an internal balloon placed within a portion of the stent to dilate the BE stabilization portion and a sheath over the stent to provide the SE Tract portion in a collapsed configuration during delivery to the site.

An alternate embodiment for the present invention is similar to the embodiment just described except that the both the stabilizing portion and tract portion of the stent are formed as a BE stent. Forming the tract portion out of a BE stent rather than a SE stent does not allow it as much flexibility during contractions of the heart. The BE stent used in this embodiment allows this embodiment to be delivered via an expandable balloon or other mechanical expansion means. The balloon can be similar to the one described for the above embodiment. The BE tract portion can be designed to be smaller in diameter to ensure that it does not impinge upon the lateral wall of the LV during systole and does not excessively push the native anterior mitral leaflet into the LVOT.

In another embodiment the stabilizing portion and the tract portion of the stent can be formed with a SE character from a SE stent material such as NiTi. The stabilizing portion of the stent can be thermally formed using standard methods to create a waist that is smaller in diameter than either of the bulbs located on each end of the waist. The waist diameter is designed such that it is similar to or preferably slightly smaller than the effective diameter of the mitral annulus so that it does not exert significant force onto the annulus. Since the mitral annulus is not exactly planar and also has a slight D-shape, the perimeter of the stent waist in its fully expanded configuration should be slightly smaller than the perimeter of the annulus. The bulbs, on the other hand, should be designed to have a 2-10 mm larger diameter than the effective diameter of the annulus (effective diameter=diameter of a circle with the same perimeter as the annulus). The bulbs will prevent migration of the device into the LA and into the LV. Delivery of the valve of this embodiment is accomplished by removal of an external sheath that holds the stent into a smaller nondeployed configuration during delivery to the site of implantation.

In yet another embodiment the valve of the present invention comprises two components that are delivered separately. The first component comprising a BE or SE stabilizing stent and a housing is delivered first to the location requiring a replacement valve via a balloon and sheath as described in an earlier embodiment. The BE stabilizing stent has a waist that is expanded adjacent the mitral annulus; the stabilizing stent has a bulb located above the waist in the LA and a bulb located below the waist in the LV. The bulbs have a larger diameter that the waist to prevent migration of the valve; the waist diameter is slightly smaller than the effective diameter of the annulus. The BE stabilizing stent is attached to a housing that is formed from a fabric such as Dacron weave, ePTFE film, or other thin flexible material that is strong, biocompatible, non-biodegradable, preferably microporous for tissue adhesion, but not allow blood flow to pass through its wall. Attached to the wall of the housing in an axial direction and spaced apart along its perimeter are a series of 6-20 axial fibers. The axial fibers can be metal such as stainless steel wire or NiTi, monofilament strand polymer such as PET, for example, or other metal or polymer strand that does not easily bend along its axis due to forces associated with blood pressure and flow. The housing can comprise metal portions or reinforcements in addition to the axial fibers.

The housing extends between the native anterior and posterior leaflets of the mitral valve; the fabric of the housing is able to fold in an axial direction between the axial fibers to act as a temporary mitral valve prior to delivery of the second component of this embodiment. The housing also serves to hold the second component (i.e., a smaller diameter stent-valve) and serve as a skirt or covering for the second component. The housing can have a conical shape to ensure that fluid forces of the blood will cause the temporary leaflets to close during systole. The housing can also have a flange located at its junction with the stabilizing stent. The flange allows the housing to have a significantly (approx. 25%) smaller diameter than the stabilizing stent and reduce the likelihood of interference with the native anterior leaflet.

The fabric of the housing can also be formed from a material that provides some elastic expansion in a radial direction; such materials include polyurethane, silicone, spandex, or other materials. Upon implantation of a stent-valve or a TAVR device into the lumen of the housing, the housing in some embodiments can expand outwards by 10-30% to provide a uniform and leak-free contact with the stent-valve and also to make contact with the stabilizing stent of some embodiments of the present invention. The housing can be formed from a metallic stent frame, for example, that is either joined to or contiguous with the stabilizing stent.

The second component of this embodiment is a SE or BE tract stent or stent-valve with replacement leaflets attached on its inner surface. The second component of this embodiment can also be a stent-valve having a smaller diameter by at least 20% than the diameter of the mitral valve annulus or the diameter of the waist of the stabilizing stent. The stent-valve can be a TAVR device that would normally be used for aortic valve replacement or a TAVR device or a stent-valve with a diameter similar to that used for TAVR. A TAVR device can be modified somewhat to work with the housing of the present invention. For example, the skirt or covering of a TAVR device could be modified or removed if the covering of the housing could serve to provide the function of preventing leakage of blood past the valve leaflets of the TAVR device or stent-valve device. The second component is delivered after the first component has been successfully positioned and attached across the mitral annulus. The second component is positioned such that the SE or BE stent-valve is located adjacent the housing. Release of the SE tract stent or smaller diameter stent-valve is accomplished by removal of an external sheath that was holding the SE stent and its contained replacement leaflets in a collapsed configuration. The second component could in an a modified embodiment be formed instead from a BE stent and delivered to the housing mounted onto a balloon shaped to fit within the housing.

The diameter of a mitral valve annulus is typically 31 mm and ranges from 27-35 mm in most patients; some patients could have an enlarged mitral valve annulus that is larger than 40 mm. The stabilizing stent of the present invention has a waist that is located adjacent the mitral annulus and is approximately 31 mm for an average diameter to match the diameter of the mitral annulus. The aortic valve annulus is significantly smaller than the mitral valve annulus with an average diameter of approximately 24 mm and ranging from 19-29 mm. The use of a smaller diameter device such as a 24 mm diameter TAVR device or stent-valve for a mitral valve replacement (TMVR) would require approximately a 20% reduction in the diameter below that of the mitral valve annulus or below that of the waist of the stabilizing stent. The advantages of using an established and developed TAVR device for mitral valve replacement are clear cost and development time advantages. Other advantages include the lower profile associated with the smaller diameter device, less impingement on the anterior mitral valve leaflet, and less impingement on the LV lateral wall.

A greater reduction in diameter for the tract stent or stent-valve that is placed within the housing smaller in diameter than that of the mitral annulus or waist of the stabilizing stent is also possible. For example the cylindrical portion of the housing of the present invention could be up to 50% smaller than the diameter of the mitral valve annulus to provide a housing for a stent-valve with a smaller diameter that is similar to that used for TAVR procedures. For example, a patient with a 40 mm diameter mitral annulus could use a 20 mm cylindrical portion diameter for the housing that flairs up to 40 mm to meet the diameter of the annulus and meet the stabilizing stent having a 40 mm stabilizing stent waist diameter.

In still another embodiment a BE stabilizing stent portion having a smaller diameter waist and two larger diameter bulbs is attached across the mitral annulus as described earlier. In this embodiment a SE tract portion is located upstream of the stabilizing portion and is located in the LA. The SE tract portion contains replacement leaflets and a covering as described earlier; the covering can extend onto and include covering the stabilizing stent.

Placement of the tract stent in the LA offers the advantage that no structure of the valve device is impinging upon the native anterior mitral leaflets and pushing it into partial obstruction of the LVOT. Additionally, no structure is located adjacent the lateral wall of the LV which could interfere with contraction of the LV.

Location of the replacement leaflets and the tract portion of the stent in the LA causes the volume contained within the LV to be somewhat larger than it was when the native mitral leaflets were working and located at the mitral annulus. It is therefore important to make the structures of the tract portion of the stent, including its covering, from materials that will not stretch upon each contraction of the heart. Even though the effective volume of the LV has increased, the cardiac output will not be adversely affected by placing the replacement leaflets in the LA as long as the materials for construction of the tract stent and its covering do not expand under systolic pressure. To help minimize the amount of expansion of the tract portion of the stent, the tract portion can be alternately formed from a BE material and can be contiguous with the BE stabilizing stent portion. Alternatively, strong reinforcement fibers can be used to prevent expansion of the tract portion of the stent.

In still yet another embodiment the tract portion of the stent containing the replacement leaflets can be located in the LA upstream of the stabilizing portion, but in this instance, both stent portions are formed from a SE material such as NiTi. The stabilizing portion can have a structure similar to the SE stabilizing stent portion described earlier. A covering can be placed along the tract portion or both portions of the stent. The SE stent portions of this embodiment allow the device to be delivered via an external sheath; the advantages can include a lower profile and the ability of the device to begin operating as a replacement valve without obstructing flow during its implantation. The SE tract portion can have a generally curved or rounded shape to ensure that contact of the tract stent with the walls of the LA are atraumatic. To help minimize the amount of expansion of the tract portion of the stent, the tract portion can be alternately formed from a BE material and can be joined with the SE stabilizing portion via means described earlier.

In a further embodiment a stabilizing stent portion having a smaller diameter waist located adjacent the annulus and two larger bulbs, one located above and another located below the annulus are formed from either a BE material or a SE material. Located within the stabilizing stent and attached to its inner surface along the waist are replacement leaflets. The smaller diameter of the waist provides for a reduction in the diameter for blood flow and hence reduced surface area for the valve material; one benefit is a lower profile for the device.

In a still further embodiment the previous embodiment can be formed such that the waist is formed from a BE material while the bulbs are each formed from a SE material. In this embodiment, the waist is not exposed to an outward force while the regions both above and below the waist exert an outward force; the upper bulb ensures that the device does not migrate toward the LV; the lower bulb ensures migration does not occur toward the LA and also holds the native mitral leaflets outwards.

Replacement of the native mitral valve with a TMVR device that has a smaller diameter than the native mitral annular diameter for blood flow offers several advantages. First the smaller diameter TMVR device will allow the lateral wall of the left ventricle to contract without hitting the TMVR device. Secondly, the TMVR device will not intrude upon the LVOT by pushing the anterior leaflet of the mitral valve into the LVOT. Thirdly, the smaller diameter TMVR will allow blood flow to occur between the TMVR and the lateral wall of the left ventricle and ensure that moving blood maintains the surface of the TMVR and native leaflets free of thrombus that could otherwise embolize resulting in a stroke.

Another embodiment of the present invention is an adapter that allows placement of an existing BE or SE TAVR device, or a stent-valve that could be used for aortic valve replacement, or a stent-valve that is at least 20% smaller than the mitral valve annulus, to be used as a component of a transcatheter mitral valve replacement (TMVR) system. This embodiment has a stabilizing portion that attaches to the mitral valve annulus and a housing portion that provides a housing or holding structure into which an existing TAVR device or smaller stent-valve can be implanted as a stent-valve that provides a blood-flow tract with a valve leaflet such as a tissue valve leaflet. The advantages of this adapter invention include that the smaller diameter tract stent or stent-valve can have a smaller profile for delivery than one with a diameter similar to a mitral valve diameter, it will not impinge onto the anterior mitral valve leaflet, and will not impinge upon the lateral LV wall. The adapter provides for a temporary mitral valve function after it has been attached across the mitral valve annulus (making the native mitral valve non-functional) until the TAVR device or other permanent stent-valve has been implanted within the housing of the adapter.

The present embodiment can be considered the first component of two-component systems described herein, where the second component comprises a permanent stent-valve such as a TAVR-type device. In one embodiment a stabilizing stent is either a BE or SE stent that wraps around the mitral valve annulus such that the stent has a larger diameter upper bulb above the mitral annulus and within the LA, and lower diameter lower bulb below the mitral annulus within the LV, with a smaller waist which is located between the upper and lower bulbs; a housing provides a place for secure placement of the second component. Another embodiment of the stabilizing stent has an inner stent and an outer stent component that can be either SE or BE in any combination. The outer stent component or tissue-contact component makes contact with tissue at or near the mitral valve annulus to hold the stabilizing stent from migration either toward the LV or toward the LA. The inner stent component or small-diameter stent component is attached to the outer stent component and provides a smaller diameter inner surface onto which a housing portion of the adapter can be attached. The smaller diameter inner surface has a diameter that is at least 20% smaller than the mitral valve annulus and at least 20% smaller than the waist of the stabilizing stent and the upper and lower bulbs. The inner surface can provide some functions of the tapered or flange portion of the housing found in some embodiments herein. In yet another embodiment of the stabilizing stent a single stabilizing stent component has two portions, one portion makes contact with the tissues of the mitral valve annulus and tissues of the LA and mitral valve leaflets to hold it from migration; another portion forms a smaller diameter inner surface with a diameter that is at least 20% smaller than the mitral annulus and provides an attachment surface for the housing component of the adapter.

The diameter of a mitral valve annulus can typically range from 27-35 mm and can exceed 40 mm in patients with enlarged hearts. The stabilizing stent of the present invention has a waist or outer waist that is similar in diameter to the mitral annulus and is approximately 27-35 mm in diameter. The diameter of waist of the stabilizing stent can be greater than 40 mm for those hearts having an enlarged mitral annulus. The aortic valve annulus diameter is significantly smaller than the mitral valve annulus with an average diameter of approximately 24 mm and ranging from 19-29 mm. The use of a smaller diameter device such as a 24 mm diameter (range 19-29 mm) TAVR device or stent-valve for a mitral valve replacement (TMVR) would require approximately a 20% reduction in the diameter below that of the mitral valve annulus or below that of the waist of the stabilizing stent. The advantages of using an established and developed TAVR device for mitral valve replacement include clear cost and development time advantages and proven safety and effectiveness. Other advantages include the lower profile associated with the smaller diameter device, less impingement on the anterior mitral valve leaflet, and less impingement on the LV lateral wall.

A greater reduction in diameter for the tract stent-valve that is placed within the housing below that of the mitral annulus or waist of the stabilizing stent is also possible. For example a stent-valve with a diameter reduction of up to 50% of the diameter of the mitral valve annulus could be used for implant into the housing of the present invention, especially for those patients having an enlarged mitral valve annulus. For example, a patient with a 40 mm diameter mitral annulus could use a 26 mm (range 20-31 mm) cylindrical portion diameter for the housing that flairs up to 40 mm to meet and attach to a waist of a stabilizing stent having a 40 mm waist diameter located adjacent the mitral valve. Alternately, the stabilizing stent can have an outer stent waist diameter that is 40 mm to make contact or near contact with the mitral annulus and an inner stent waist diameter that is approximately 26 mm (range 20-31 mm) to attach to the diameter of a cylindrical housing of 26 mm (range 20-31 mm) diameter. The cylindrical housing of 26 mm (range 20-31 mm) diameter could provide a housing for a 26 mm (range 20-31 mm) TAVR device or other stent-valve device.

The housing portion of the adapter is attached at its proximal or upstream end to the stabilizing stent; the attachment can be to the center of the stabilizing stent adjacent the annulus or it can be attached to the distal or downstream end of the stabilizing stent. The housing can be formed from a fabric material with axial fibers such that the housing performs as a temporary valve. The housing can be tapered or it can have a flange or funnel on its inlet or upstream end where it is attached to the stabilizing stent; the housing can have a cylindrical portion to provide a location for deployment and implantation of a TAVR device or other smaller diameter stent-valve within the interior surface of the housing. The housing can also be cylindrical in shape throughout and attach to the smaller diameter inner stent component of a stabilizing stent. The housing can also contain temporary valve leaflets to form a temporary valve function prior to delivery of a TAVR device or other stent-valve into the housing. The leaflets can be formed from polymer and attached to the surface of the housing. Such polymeric temporary leaflets have a thin wall that is thinner than standard tissue valve leaflets to improve the profile of the device for delivery. The temporary valve can take the form of a trileaflet valve such as the aortic valve or a bileaflet valve such as a venous valve or a duckbill valve. When the housing contains temporary valve leaflet(s), the housing itself can be more rigid, such as a metal structure similar to a stent.

Following implant of the adapter across the mitral valve annulus and within the native mitral valve leaflets, a TAVR device or other tract stent-valve is implanted within the housing. The housing does not expand appreciable thereby providing friction with the tract stent-valve to hold it in place from migration. The tract stent-valve can be a BE TAVR device or a SE TAVR device that is delivered via balloon expansion or via release from an outer constraining sheath.

The stabilizing stent or adapter for a small diameter TMVR valve device forms a tight seal with the mitral annulus to prevent leakage and prevents migration of the TMVR toward the left atrium and toward the left ventricle; a covering or skirt can be provided to help prevent leakage. The stabilizing stent for the TMVR or TMVR adapter must not push the mitral annulus outward with a continued force that would cause further mitral annular dilation for the patient with functional mitral disease. The stabilizing stent for the TMVR or TMVR adapter should allow postdilation for the patient that having stenotic mitral valve leaflets to ensure a tight fit between the stabilizing stent and the mitral annulus to ensure a good seal without leakage and without likelihood for stent or device migration. The TMVR adapter should provide full support and seal to a smaller diameter stent valve such as a TAVR device that is inserted into the stabilizing stent as a second step.

The present invention includes a structure for the wall of a SE stent, a portion of the wall of a SE stent, or a stent portion of a stented device that allows a lower outward force to be applied to the tubular vessel of the body into which it is being delivered. The stent is well suited for use as a SE stent component in a TAVR device as well as a TMVR device. The invention can be used for stents, for example, for coronary artery treatment, peripheral artery or vein treatment, or in treating other tubular vessels or orifices of the body. Due to the lower outward force from the present invention in its expanded configuration, the tubular body vessel will be exposed to less trauma and will have less likelihood of the stent struts migrating through the tubular tissue wall or otherwise causing undesired complications. The lower outward force after deployment reduce the outward force applied against a catheter lumen or the delivery sheath when the stent is constrained during delivery to the site of the lesion. Once the SE stent is delivered to the site of the lesion, a post dilation can be provided to place the stent into greater contact with the wall of the vessel; this post dilation activates BE elements of the stent to expand the stent in a controlled manner, without exposing the vessel wall to any significant ongoing outward forces.

The SE stent described here can be used in a stent-valve such as a TAVR device used for treatment of aortic valve stenosis, or a stent used as a component of a TMVR system. It is understood that the concepts described in the present disclosure can be applied to a SE stent or stent-valve used anywhere in a tubular vessel of the body.

In one embodiment a SE stent has an open cell structure that can have, for example, at least some of the components of the stent wall structure in the shape of zig-zag stent or similar open-cell stent structure found commonly in current stents used throughout the body. The stent is comprised of hinge regions (or hinges) and strut regions (or struts); the hinges are the curved regions of the stent and undergo the vast majority of the deformation as the stent expands outwards during expansion deformation either via a balloon or via elastic energy storage within the hinge that is released in part during expansion of the stent. The struts are typically linear regions that connect two or more hinges together to form the stent wall structure. In the present invention some of the hinges are formed from a BE or plastically deformable material or have a BE shape; other hinges are formed from a SE or elastically deformable material or have a SE shape.

As the zig-zag stent is collapsed to a smaller diameter configuration for delivery to the lesion site within a constraining sheath, the SE hinges collapse elastically but the BE hinges are retained in a collapsed configuration and do not significantly deform while the stent is being constrained, nor do they significantly deform later when the stent is released from the constraining sheath and allowed to expand elastically into the vessel of the body. As the SE hinges deform to allow the stent to expand outwards upon release from the delivery sheath, the outward force that they generate drops off as the diameter of the stent enlarges. The outward force generated by the stent of the present invention is lower in its initial expanded state after release from the constraining sheath than by a normal SE stent. This lower force is acceptable since the stent of the present invention is not required to retain as much outward force after it is release against the tubular tissue and is not required to attain as large of a diameter in its expanded configuration in free space (i.e., no constraining member) as a normal SE stent. The lower outward force of the present invention generates less trauma to the tissues of the tubular vessel and for the TAVR application it results in less formation of heart block. For a TMVR application, this lower outward force avoids significant dilation of the mitral annulus, or reduces the displacement of the native anterior mitral leaflet which could interfere with the LVOT, for example. For other applications in tubular vessels or orifices in the body, the lower outward force can similarly reduce trauma to the tissues of the vessel, or reduce strut migration through the tissue, for example.

The stent of the present invention is then able to be further expanded via a post-dilation from a dilation balloon or other expansion means after it has been partially deployed via expansion of its SE hinges but not fully deployed via its non-deployed BE hinges. The BE hinges found in the zig-zag wall structure can be expanded to allow the stent to undergo a second expansion or post-dilation to a larger diameter. This second expansion causes the BE hinges to undergo a plastic deformation to place the stent into a greater contact with the wall of the tubular vessel to reduce the likelihood for perivalvular leaks or stent-valve migration. This secondary expansion does not generate a continued outward force onto the tubular vessel like other SE stents but instead is expanded outwards in a manner similar to other BE stents; these outward forces imposed by the stent onto the tubular tissue will reduce over a short period of time (i.e., hours or days) and will not result in long term need for a permanent pacemaker (due to continued forces normally imposed by other SE stents onto the bundle of HIS) in the case of a TAVR application. In a TMVR application, this will avoid significant outward force and continued dilation of the mitral annulus.

To provide adequate strength to the BE hinges, they can be constructed with a greater height or a larger dimension in the radial direction than that of the struts. In this manner the hinges can retain their closed curved shape at a small radius of curvature (RC) as the stent is being forced into the constraining sheath for delivery and also retain the small RC after the stent has been released from the constraining sheath.

The wall structure of the present invention can also have a closed cell structure. The closed cell structure contains at least some BE hinges and some SE hinges. The BE and SE hinges are comprised of at least some series arrangement of BE hinges with the SE hinges. The BE and SE hinges can be arranged within the wall structure of the stent such that the stent can be compressed to a smaller diameter configuration via elastic compression of some or all of the SE hinges as it is constrained within a delivery sheath; the stent can be released from the constraining sheath and the SE hinges can expand outward via elastic expansion to a first enlarged diameter. The presence of additional BE hinges located in series with the SE stent wall portion allows the wall structure to be further expanded via a balloon expansion to achieve a diameter that is an even larger second diameter to allow the stent to come into intimate contact with the surrounding tissues. The enlarged stent of the second larger diameter does not provide the continued outward force that is typically found with a standard SE stent.

An expansion limiter can be located to subtend from one strut to another across a SE hinge; the expansion limiter is a metallic or polymeric connection that is easily bent but is strong in holding tension as the SE hinge is expressing an outward force to try to cause further expansion of the SE hinge. The expansion limiter can provide two characteristics to the stent wall structure of the present invention. First, the expansion limiter prevents the SE hinge from expanding outwards after the SE hinge has expanded a specified set amount and hence the SE hinge does not provide a continual outward force against the wall of the tubular vessel. Secondly, the expansion limiter prevents the SE hinges from undergoing further expansion during a post dilation of the stent via a balloon or mechanical expansion means; the expansion of the BE hinges due to the post dilation are hence required to expand without any further expansion of the SE hinges.

The BE hinge of the present invention can be formed from an elastic material such as NiTi or a generally plastic metal material such as stainless steel or polymer as long as it is formed with a shape that provides plastic character to the BE hinge. The BE hinge should be formed with a hinge length that is short in comparison to that of a SE hinge by a factor of at least two. In this manner the BE hinge will focus the expansion deformation and will provide a plastic deformation during expansion deformation. The hinge height in the radial direction should be larger than the height of the struts such that the hinge has enough strength such that it does not deform during the compression of the stent into the delivery sheath. A hinge height of approximately at least two times the strut height will provide the necessary strength to resist deformation of the BE hinge within the delivery sheath. The BE hinge is contained within the delivery sheath in a bent configuration having a small radius of curvature. The width of the struts adjacent the BE hinge is at least twice the width of the BE hinge to obviate expansion deformation of the strut and thereby causing expansion deformation to occur in any BE hinges adjacent the strut.

The SE hinge of the present invention can be formed from an elastic metal material such as NiTi or a generally plastic metal material such as stainless steel as long as it is formed with a shape that provides an elastic character to the SE hinge. Note that for other lesser strength applications such as for stents used in the coronary artery or peripheral artery, the material can be a polymeric or a biodegradable material. The SE hinge should be formed with a hinge length that is long in comparison to that of a BE hinge by a factor of at least two. In this manner the SE hinge will not focus the expansion deformation and will provide an elastic deformation during expansion deformation. The hinge height in the radial direction should provide the necessary outward force to the stent to ensure that it expands the stenotic tissue of the tubular vessel outwards. The SE hinge is contained within the delivery sheath in a bent configuration having a small radius of curvature and expands outwards upon release from the constraining sheath to a larger radius of curvature.

The stabilizing stent of the present invention is a component of a TMVR system. The stabilizing stent forms an attachment to the mitral annulus or across the mitral annulus. In several embodiments the stabilizing stent also is attached to a housing that contains temporary mitral valve leaflets; such stabilizing stent is a part of an adapter for the TMVR system. A smaller stent valve such as a TAVR device can be placed into the housing as a second step. In other embodiments the stabilizing stent is attached to the permanent replacement mitral valve leaflets and the TMVR device is implanted as a single step. Some embodiments are intended for patients with functional mitral regurgitation and other embodiments are intended for patients having congenital or primary mitral valvular disease. The stent structure, for example, can include open or closed cell structure, configurations of hinges and struts, presence of connectors between stent rings, etc. The stent structure can be similar to the stent structures found in stents currently being used in the body.

In one embodiment the stabilizing stent is formed from an open or closed cell wall structure consisting of a series of rings having a zig zag pattern found in many cardiovascular or coronary stents. The stabilizing stent is formed from SE materials such as NiTi or other elastic materials. The stent is formed such that upon release from a delivery sheath the upper bulb and lower bulb form a larger diameter than the waist which is placed in line with the mitral annulus in an axial direction. The bulbs and waist can be formed or laser cut from a tubing such that the length of the struts of the bulb are similar to each other but are significantly longer than the struts of the waist portion. Alternately, the angle of the struts of the waist portion can be of a greater angle with respect to the stabilizing stent axis than the angle of the struts of the upper and lower bulbs.

In another embodiment the stabilizing stent has the bulbs formed from a SE material in a series of zig zag rings. The waist is formed with a zig zag ring pattern wherein some of the hinges are BE and other of the hinges are SE; this combination BE/SE waist portion of the stabilizing stent is able to expand outwards upon release from the delivery sheath but can be expanded further outwards via a balloon or other expansion means to form a larger diameter. The SE hinges of the waist portion can contain expansion limiters as described earlier to ensure that the BE hinges are plastically deformed during any further expansion of the waist. If this embodiment is used as a stabilizing stent for an adapter wherein further implant of a stent-valve occurs within the stabilizing stent, the combination BE/SE waist portion allows the stent-valve to seat within the waist of the stabilizing stent and securely hold the stent-valve via its BE characteristics.

Still another embodiment for the stabilizing stent is a pinching stent that shortens axially in a pinching motion on the inlet and outlet sides of the mitral annulus as it is expanded outwards in diameter. This stabilizing stent has a larger proximal bulb and distal bulb and a smaller waist portion. Delivery of this stabilizing stent can occur by containing the open ends of the stent within a cone and body of a delivery sheath.

In yet another embodiment the stabilizing stent is again formed from a series of open cell or closed cell ringlets of zig zags that are generally connected via connecting members (i.e., flexible strut-like elements that are typically easily deformed) and covered with a material that does not allow free passage of blood though its wall. The stabilizing stent of this embodiment is formed with an inner waist stent and an outer waist stent along with an upper and lower bulb stent portions. The stabilizing stent serves as a support or adapter for further implant of a stent-valve within its interior. The outer waist stent is a combination SE/BE stent as described earlier to allow BE contact of the outer stent with the mitral annulus. The inner waist stent is a SE stent that allows post dilation of the outer stent outwards to meet the mitral annulus (if necessary) but elastically contracts back to a smaller diameter to allow to match the diameter of a smaller diameter stent-valve that can be implanted within its interior as a second step. The outer waist stent acts as a BE stent such that it does not apply continued outward forces against the mitral annulus; such a stabilizing stent has definite application in treating functional mitral disease. The stabilizing stent component of the adapter along with the adapter and temporary valve leaflets can be delivered via a delivery catheter in a manner similar to a standard SE stent. Temporary leaflets are attached to the stabilizing stent in a manner similar to any of the embodiments of the present invention described in this application.

In a further embodiment for the stabilizing stent the waist portion is formed from an inner and outer waist stent. The outer waist stent is a SE stent and the inner waist stent is a BE or combination SE/BE stent having expansion limiters. The stabilizing stent is delivered by release from a delivery catheter in a manner similar to that of standard SE stent delivery. The bulb portions of the stent locate on each side of the mitral annulus. The outer waist stent is expanded outwards against the mitral annulus and provides a continued outward force against the mitral annulus; this outward force is well tolerated by patients with congenital mitral leaflet disease, mitral valve prolapse, or for patients with stenotic mitral valve leaflets. Once the stabilizing stent is located in place across the mitral annulus, and the temporary valve leaflets are functioning properly, the smaller diameter stent-valve, such as a TAVR device or modified TAVR device can be placed within the inner waist. The inner waist can expand in diameter to accommodate the small diameter stent-valve implanted within its interior. The BE portion of the inner waist can expand outwards to form a restraining force that will hold the TAVR device from embolizing. Expansion limiters located across the SE hinges can prevent further SE member expansion during TAVR placement within the inner waist.

An important aspect of TMVR design is to ensure that blood can flow across all free surfaces and reduce the amount of thrombus formation that occurs that can result in emboli particles that may result in strokes or other embolic complications.

In another embodiment, a smaller diameter stent-valve is attached to the stabilizing stent such that both the stabilizing stent and the stent-valve are delivered in one step. The stent-valve is attached to the stabilizing stent near its inflow end. The permanent valve leaflets attached to the stent structure of the stent-valve are attached in a crown-shaped pattern that provides open areas in the stent of the stent-valve that allow free blood flow across the walls of the stent-valve frame but not across the leaflets. This blood flow through the walls of the stent-valve frame ensure that the inside surface of the native mitral leaflets are maintained free of thrombus. The smaller diameter of the stent-valve allows blood flow behind the native mitral leaflets and ensures that thrombus does not collect on the outer surface of the native leaflets.

In yet another embodiment the housing for the adapter device is formed with a conical shape having the smaller diameter of the cone nearest the outflow end of the housing. The conical housing allows the native valve leaflets to lie against the outer surface of the housing cone in a stable position. During systole, the blood pressure and flow generated in the left ventricle push the native leaflets against the outer conical surface of the housing where the leaflets can eventually attach and heal. Blood flow cleanses the outer surface of the native leaflets during systole. The conical surface also provides clear and free access for blood flow out of the LVOT. A similar conical design for the housing of a stent-valve that is permanently attached to the stabilizing stent is also anticipated.

In another further embodiment the stabilizing stent of the present invention can have a cylindrical waist portion that extends for a length of the temporary mitral valve leaflets (approx. 0.75-1.0 inches). This cylindrical portion is the inner waist stent and is attached to the outer stent. The inner stent is a combination BE/SE stent or a SE stent with expansion limiters. The outer stent has a waist portion that is SE and two SE bulbous portion on each side of the waist portion. A set of temporary mitral leaflets is attached to the wall of the inner stent along its length. After implantation of this temporary adapter, a stent-valve having a smaller diameter, such as a TAVR device or a modified TAVR device is implanted within the cylindrical portion of the inner waist stent.

An additional embodiment for the stented mitral valve or adapter valve replacement apparatus of the present invention comprises a fixation means to attach the stabilizing stent to the mitral annulus or the mitral leaflets. In one embodiment the waist region of the stabilizing stent comprises multiple barbs for fixation that are attached to the stent via a BE hinge. Upon release of the stented valve across the mitral annulus the generally SE stent or combination stent having both BE and SE hinges expands outwards to contact the mitral annulus. For the combination BE/SE stent further balloon dilation of the stent can force the stent into more immediate contact with the mitral annulus. After delivery of the SE stent or combination BE/SE stent, a dilation balloon within the lumen of the stent is inflated to push the barbs outwards. The barbs pivot about the BE hinge and are driven into the mitral annulus or the mitral leaflet along the perimeter of the mitral valve.

Various configurations of the fixation means have been contemplated, including penetrating barbs, grippers and combinations. In some embodiments, barbs are attached to the stent at junction points (which can be, but are not required to be, hinges) that connect various strut elements. The barbs can be located principally along the waist of the stabilizing stent or they can alternatively be located in the lower bulb region of the stabilizing stent where contact and attachment can be made near the base regions of the native mitral valve leaflets such as approximately 1-5 mm from the leaflet attachment to the mitral annulus. The barbs can be oriented such that they undergo a directional movement during activation by the dilation balloon to move them in an axial and radial direction. An alternate configuration for the barbs places them in a more circumferential orientation around the perimeter of the stent. Dilation of the stent causes these barbs to move both radially outwards and also circumferentially during activation of the barbs.

The barbs can be formed from a bistable geometry (such as having a thin-walled spherical section) that allows the barb to assume either of two equilibrium positions; one position has the barb located in a nondeployed position such that it does not extend beyond the outer perimeter of the stent during delivery in the sheath and also after release from the sheath. Upon exposure to a specified balloon pressure of the dilation balloon, the barb moves from one bistable state in the nondeployed position to a another bistable state in the deployed position that extends the barb outwards and into the tissue of the mitral annulus or mitral leaflet. The benefit of this barb design is that the barb would not be deployed into the tissue until the stent is fully positioned across the annulus and then the barb is activated and pushed into the surrounding tissue to anchor or attach the stent against migration when desired; further, the barb acts to hold the mitral tissue inwards and to help prevent the potential continual enlargement of the mitral annulus thereby reducing the likelihood of later development of perivalvular leaks around the stented mitral valve.

In one embodiment the mitral stent valve or adapter of the present invention comprises either a SE stabilizing stent or a combination BE and SE stent portion. The mitral valve or adapter of this construction is generally delivered through a sheath by a pusher and expands outwards upon expansion release from the sheath. In one embodiment the upper bulb of the stabilizing stent further comprises recapture elements such as struts or filaments that are attached to the upstream end of the upper bulb. The recapture struts can be generally longer in axial length than other struts of the stabilizing stent to allow the stabilizing stent to be released from the sheath (except for portions of the recapture elements) but still be releasably held to the pusher via the recapture struts. The pusher can be retracted within the sheath to allow the stent valve to be reintroduced back into the sheath after the stent has been expansively released. In this way, the stent valve or adapter can be repositioned so that the waist of the stabilizing stent is located adjacent to the mitral annulus, or removed completely if necessary. The attachment of the recapture struts to the pusher can then be released to allow a full release of the stent valve when the position of the valve is correctly positioned across the mitral annulus. In some embodiments, the recapture elements comprise filaments releasably attached to the upper bulb of the stabilizing stent. In some embodiments, the recapture elements comprise struts or wires which are fusibly attached to the upper bulb of the stabilizing stent; when the position of the stent valve or adapter is determined to be satisfactory, fusible connection portions are broken such as by thermal, electrical, or mechanical means. In some embodiments, the recapture elements comprise mechanical attachment mechanisms such as clamping features, jaws, interference fits, threaded or geared elements, and so forth.

The pusher can be formed from a hollow tube such that upon expansion release of the stent valve from the sheath, a dilation balloon catheter can be introduced through the hollow pusher and placed within the stented mitral valve. Expansion of the balloon can then cause the barbs located in the waist or lower bulb of the stabilizing stent to be activate and push into the annulus tissue or leaflet tissue. After activation of the barbs while the replacement valve or adapter is held in the proper position securely fixes the replacement valve or adapter in position, the recapture struts can be detached from the hollow pusher. The attachment of the recapture struts to the pusher can be in the form of a cord that is released, a screw mechanism, a clasp mechanism, or any mechanical, thermal, or electrical release mechanism that would release the recapture struts from the pusher.

One key aspect and feature of the present invention provides an adapter which allows transcatheter implantation of a replacement mitral valve which is smaller than the native valve, reducing the delivery profile of the replacement valve, and allowing the use of TAVR valve devices which are well developed and readily available to be used in the larger mitral annulus.

Another key aspect and feature of the present invention is structure which provides improved blood flow around the implanted device(s) and the native mitral leaflets for reduced thrombus generation and reduced particle embolization.

Yet another key aspect and feature of the present invention is a first component comprising a temporary valve to provide mitral valve function until a second permanent mitral valve replacement component is implanted.

Still another key aspect and feature of the invention is a two-step transcatheter mitral valve replacement.

A further key aspect and feature of the present invention is a stabilizing stent which does not cause further mitral annulus dilation, yet securely anchors the replacement mitral valve in position at the mitral annulus.

A still further key aspect and feature of the present invention is a combination balloon-expandable and self-expanding functionality in a single stent structure.

Still an additional key aspect and feature of the present invention is a structure which provides controlled and limited expansion of a portion of a self-expanding stent with ongoing self-expanding elastic forces in other portions of the stent.

Having thus briefly described one or more embodiments of the present invention, and having mentioned some significant aspects and features of the present invention, it is the principal object of the present invention to provide apparatus and methods for transcatheter mitral valve replacement.

Additional objects of embodiments of the present invention include: providing lower profile transcatheter mitral valve replacement, providing combination self-expanding/balloon expandable stents for use in various applications in tubular vessels and orifices of the body, and providing an adapter for placing a smaller replacement valve in a larger annulus, and providing methods of fabrication of the stents and valve apparatus, and providing methods of treating dysfunctional mitral valves.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIGS. 1A, 1B, 1C, and 1D illustrate example valve replacement apparatus of the present invention, comprising BE and SE material.

FIGS. 2A, 2B, and 2C illustrate delivery and deployment apparatus and methods for the embodiment of FIG. 1A-1D.

FIG. 3 illustrates another example valve replacement apparatus of the present invention, comprising BE material.

FIG. 4A and FIG. 4B illustrate delivery and deployment apparatus and methods for the embodiment of FIG. 3.

FIGS. 5A, 5B, and 5C illustrate another example valve replacement apparatus of the present invention, comprising SE material, and delivery and deployment apparatus.

FIGS. 6A, 6B, and 6C illustrate another example valve replacement apparatus of the present invention, comprising two separate components, placed in two steps.

FIGS. 7A, 7B, 7C, and 7D illustrate details of embodiments of a housing for embodiments including that of FIG. 6A-6C.

FIGS. 8A, 8B, 8C, 8D, and 8E illustrate delivery and deployment apparatus and methods for embodiment such as that of FIG. 7A-7D.

FIGS. 9A, 9B, 10A, 10B, 11, and 12 illustrate examples of valve replacement apparatus of the present invention similar to that of FIGS. 1A-5C, but where the replacement valve is placed in the left atrium.

FIG. 13 illustrates another example valve replacement apparatus of the present invention where the replacement valve is placed in the left atrium, but where the tract element is curved.

FIG. 14 illustrates another example similar to those of FIG. 9A-9B, and a corresponding delivery system and method.

FIG. 15, 16, 17A, an 17B illustrate examples similar to those of FIGS. 1A-5C, but where the replacement leaflets are located in the waist, and a corresponding delivery system and method.

FIGS. 18A, 18B, 18C, 19A, 19B, 19C, and 19D illustrate an embodiment of the present invention configured as an adapter.

FIGS. 20A, 20B, 20C, and 20D illustrate a cylindrical housing for the embodiment of FIG. 18A-18C.

FIGS. 21A, 21B, 21C, 21D, 21E, 21F, and 21G illustrate further examples of adapters and housings similar to those of FIGS. 18A-20D, showing various housing configurations.

FIGS. 22A, 22B, 23A, and 23B illustrate placement of a replacement valve using the adapter and housing of FIGS. 18A-21G.

FIGS. 24A, 24B, 25A, 25B, 26A, 26B, 26C, 27, 28A, 28B, 28C, 29A, 29B, 29C, 30A, 30B, 31A, and 31B illustrate examples of self-expanding stents of the present invention which are adapted for post-dilation, and associated methods.

FIGS. 32A, 32B, 32C, 32D, 33A, 33B, and 33C illustrate examples of stents of the present invention incorporating features shown in FIGS. 24A-31B, which are adapted for stable implantation at a valve annulus and providing for additional components to be implanted therein, and associated methods.

FIGS. 34A, 34B, 34C, 34D, 35A, and 35B illustrate an example stent of the present invention adapted for pinching or grasping the valve annulus, and associated delivery apparatus and methods.

FIGS. 36A, 36B, 37A, and 37B illustrate stabilizing stents of the present invention having inner and outer portions.

FIG. 38 illustrates valve replacement apparatus of the present invention adapted with stabilizing stents such as those of FIGS. 36A-37B.

FIG. 39 illustrates another embodiment of the present invention having a conical tract portion.

FIGS. 40A and 40B illustrate an embodiment of valve replacement adapter of the present invention including temporary or permanent replacement leaflets.

FIGS. 41A, 41B, 41C, 42A, 42B, 43A, and 43B illustrate additional embodiments of valve replacement adapter configured for low profile delivery with subsequent delivery of a smaller replacement valve than the native valve.

FIGS. 44A, 44B, 44C, 45A, 45B, and 45C illustrate alternate configurations for the waist of a stabilizing stent for an adapter such as those of FIGS. 40A-43B.

FIGS. 46A, 46B, 46C, 46D, 46E, 46F, 47A, 47B, 47C, 47D, 47E, 48, 49A, 49B, 49C, 49D, 49E, 49F, 49G, 49H, 50A, 50B, 51A, 51B, 51C, and 51D illustrate configurations for attachment means to secure the device in position.

### DETAILED DESCRIPTION OF THE PREFERRED EMBODIMENTS

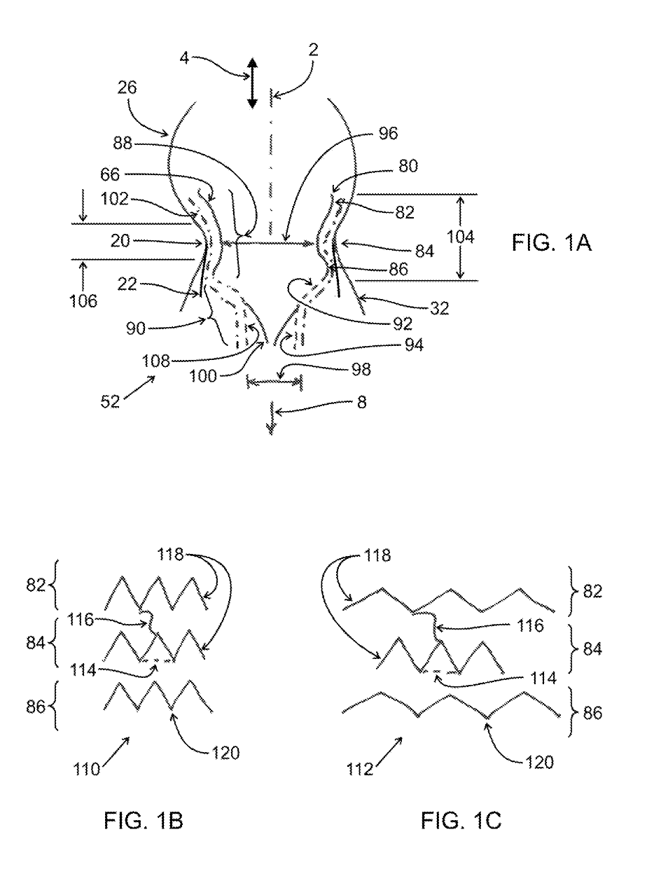

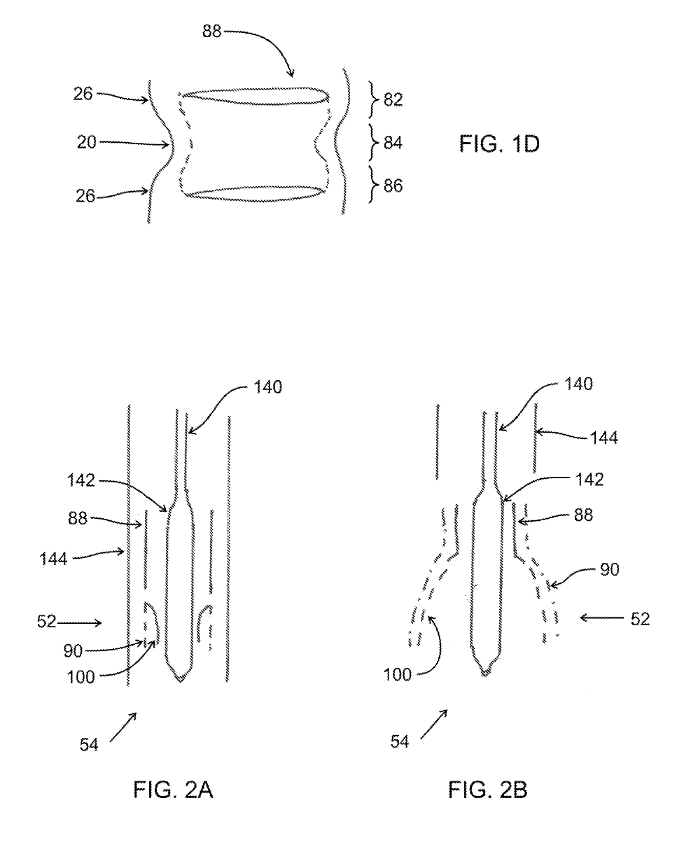

One embodiment of the valve of the present invention is shown in FIGS. 1A-1D. Replacement valve 52 has a mitral annular holding structure or stent 66 comprising stabilizing portion 88. Replacement valve 52 also comprises tract element 90. Stabilizing portion 88 of stent 66 for this embodiment is formed from a BE material such as stainless steel, cobalt alloy, or other materials, including polymeric materials used for forming stents; the BE material can also be NiTi or other elastic metals used in medical stents that is machined to form a focused region at the expansion sites that will deform plastically. Stabilizing portion 88 has a waist 84 with a waist diameter 96 that is somewhat smaller than the mitral annulus 20 to which it is positioned adjacently. A somewhat enlarged region or upper bulb 82 is joined to waist 84 and is of a larger diameter by approximately 2-10 mm than waist 84; the upper bulb 82 preferably has a free end 80 which is curved toward the centerline or axis 82 of stent 66 to avoid abrasion due to contact with the left atrial wall 26. Another somewhat enlarged region or lower bulb 86 of stabilizing portion 88 is also larger than waist 84 by approximately 2-10 mm and makes direct contact with native mitral leaflets 22 to push them outwards near mitral annulus 20 by approximately 2-10 mm. Stabilizing portion 88 has an axial length 104 of approximately 6-20 mm and waist 84 has an axial length of approximately 5-10 mm. FIG. 1A, and many other similar views of various embodiments, illustrates a cross section through various elements for clarity of illustration; the elements typically are approximately axisymmetric, so that rotating the illustrated portions around approximately in a circular path would construct the complete elements, parts, anatomy, or embodiment. Certain features can deviate from this general configuration as noted, such as the LVOT and the left ventricular lateral wall, or having 3 leaflets, and so forth. Also, the native anatomy and therefore elements of various embodiments intended to be placed within the native anatomy, is known to be not perfectly circular, but can be somewhat oval, or somewhat “D”-shaped. Other than the one, two, three, or other number of leaflets of temporary and permanent replacement valves of various embodiments herein, the other elements of the invention are typically symmetric when in their delivery configuration during advancement into position, but can take on a somewhat out-of-round shape to match the native anatomy when deployed and functioning for valve replacement. The native anatomy may also reshape somewhat when the various components of embodiments of the present invention are deployed, tending towards somewhat more round configuration.

Stabilizing portion 88 of stent 66 is attached or joined to tract element 90. Tract element 90 can be a portion of stent 66 as illustrated in FIG. 1, or can be a housing as illustrated in other figures and further described below, which can be a separate tubular element such as a cylinder, for example, that is attached to the stabilizing portion and is able to hold or house valvular leaflets or house a separate stent-valve that contains valve leaflets. In this embodiment, tract element 90 is formed from a SE material to allow for bending and movement of the tract element 90 as it comes into contact with left ventricular (LV) lateral wall 32. The SE material of tract element 90 provides reduced abrasion of the LV lateral wall 32 during systolic contraction of the heart. Tract element 90 has a tapered region 92 that extends from a diameter 2-10 mm larger than the mitral annulus 20 to a diameter that is approx. 5-15 mm smaller than the mitral annulus 20. A mitral annulus can range in effective diameter from approximately 30-45 mm in a typical adult. The diameter for the cylindrical region of tract element 90, tract diameter 98, is approximately 15-25 mm. Contained within tract element 90 and in some embodiments, within cylindrical region 94, are replacement leaflets 100. Replacement leaflets 100 are attached to the inner surface 108 of tract element 90. Leaflets 100 are attached to inner surface 108 along a curved path or crown-shaped path similar to the attachment line of native trileaflet cardiac valves such as natural aortic valve leaflets attached to the wall of the aortic sinus. Replacement leaflets 100 can be formed from tissue materials such as pericardium or from synthetic materials such as polymeric materials. Replacement leaflets 100 can be attached to tract element 90 via sutures, adhesive, thermal means, or other attachment means. Covering 102 is placed around tract element 90 and attached to it via sutures, adhesive, thermal methods, or via other attachment means. Covering 102 is intended to direct blood flow through replacement leaflets 100 during diastole and not allow blood to pass around replacement leaflets 100 during systole. Covering 102 can extend around stabilizing portion 88 of stent 66, if desired, to ensure that blood does not find a leak path around replacement leaflets 100, which could result in a perivalvular leak around replacement valve 52. Covering 102 can comprise a Dacron® or other polyester weave, knit, or fibrous fabric, or other polymeric fabric, or ePTFE, or a composite material fabric. Covering 102 can comprise a fibrous polymeric mat, foam layer; covering 102 is preferably porous, with small porosity to allow tissue ingrowth but limit or eliminate significant blood leakage, but can be filled with or coated with a degradable or non-degradable material to reduce leakage early after implant but allow later tissue ingrowth, or can be non-porous. Covering 102 in some embodiments can be formed from an elastic material such as polyurethane, silicone, or spandex; such elastic covering materials can allow covering 102 to expand uniformly as stent 66 and/or tract element 90 to which it is attached is expanded outwards in a radial direction during delivery and deployment of the apparatus.

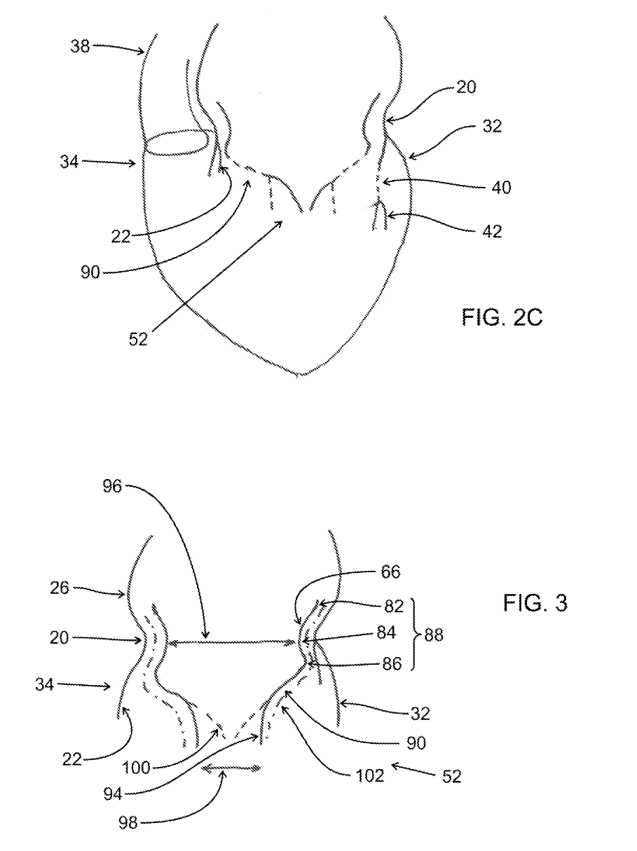

As shown in FIGS. 1B-1D stabilizing portion 88 of stent 66 can have one or more expansion limiter(s) 114 that limit waist 84 from expansion beyond a set diameter; the set diameter is preferably less than the effective diameter (diameter of a circle with the same perimeter) of mitral annulus 20. Expansion limiter 114 is a metal or polymeric fiber or strand that connects between two or more struts 118 of stent 66 in such a way as to prevent the struts 118 of stent 66 from separating fully as stent 66 is expanded. Alternatively, an altered stent design can be used to restrict the amount of expansion one portion of a stent will expand relative to a neighboring portion. For example, struts 118 can be formed with a greater angle in a circumferential direction that restricts further expansion to the extent that neighboring struts having a lower angle can expand outwards. In another example, some struts 118 can have a shorter length between expansion sites 120, so that when those struts are reoriented during expansion, they form a smaller circumference than do other struts 118. The geometry of some expansion sites 120 can also be chosen to reduce the circumference of portions of the stent. As balloon 142 (FIG. 2) pushes waist 84 and upper bulb 82 and lower bulb 86 outwards, waist 84 remains smaller in diameter than upper bulb 82 and lower bulb 86. Upper bulb 82 and lower bulb 86 expand to a diameter that is larger than that of mitral annulus 20 and thereby grab or capture mitral annulus 20 from the LA side and the LV side.

FIGS. 1B and 1C illustrate portions of stent 66, with FIG. 1C showing stent 66 in a more expanded state. FIG. 1D illustrates a portions of stent 66, mitral annulus 20, left atrial wall 26, and left ventricular wall 30, to show how the expanded shape of stent 66 interacts with mitral annulus 20, left atrial wall 26, and left ventricular wall 30 to secure stent 66 in place.

Note that in order to better show the invention apparatus and its functions and methods, portions of the human body are described and depicted in some of the figures; these elements are for reference only and in no way form part of the invented apparatus.

FIGS. 2A-2C show the embodiment of FIG. 1 being delivered to the site requiring a transcatheter mitral valve replacement (TMVR). FIG. 2A shows stabilizing portion 88 of stent 66 temporarily affixed onto the outside of balloon 142 located at the end of delivery catheter 140 in a delivery configuration. Tract element 90 containing replacement leaflets 100 is being held in a nondeployed configuration via sheath 144. As sheath 144 is partially withdrawn, the SE stent portion begins to deploy and replacement valve 52 remains affixed to the balloon as shown in FIG. 2B. As illustrated in FIG. 2C, expansion of balloon 142 causes waist 84 of stent 66 to expand adjacent mitral annulus 20, and upper bulb 82 and lower bulb 86 to expand above and below mitral annulus 20 to a diameter greater than that of mitral annulus 20. The replacement valve of some embodiments of the present invention can be expanded with a dilation balloon 142. Balloon 142 can be a cylindrical balloon such as those known in the art. As the balloon expands to dilate the upper bulb, waist, and lower bulb of the stabilizing stent, the stent waist can restrict further expansion of the balloon at the waist. The tract portion of the stent can restrict further expansion of the distal portion of the balloon. Thus, the various portions of the stabilizing stent and tract portion can dilate expand different amounts, even when using a typical cylindrical balloon as is known in the art.

Upon deflation of balloon 142 and withdrawal of delivery catheter 140 and sheath 144 as shown in FIG. 2C, the TMVR replacement valve 52 is implanted across mitral annulus 20 and holds native leaflets 22 outwards, but not so far outwards that tract element 90 pushes a native valve leaflet into the left ventricular outflow tract 34 and blocking outflow of blood through the aorta 38, or into and interfering with the left ventricular lateral wall 32 thereby restricting left ventricular wall motion. The replacement valve 52 is deployed so that it is far enough from chordae tendinae 40 and papillary muscle 42 to avoid interfering with function of replacement valve 52.

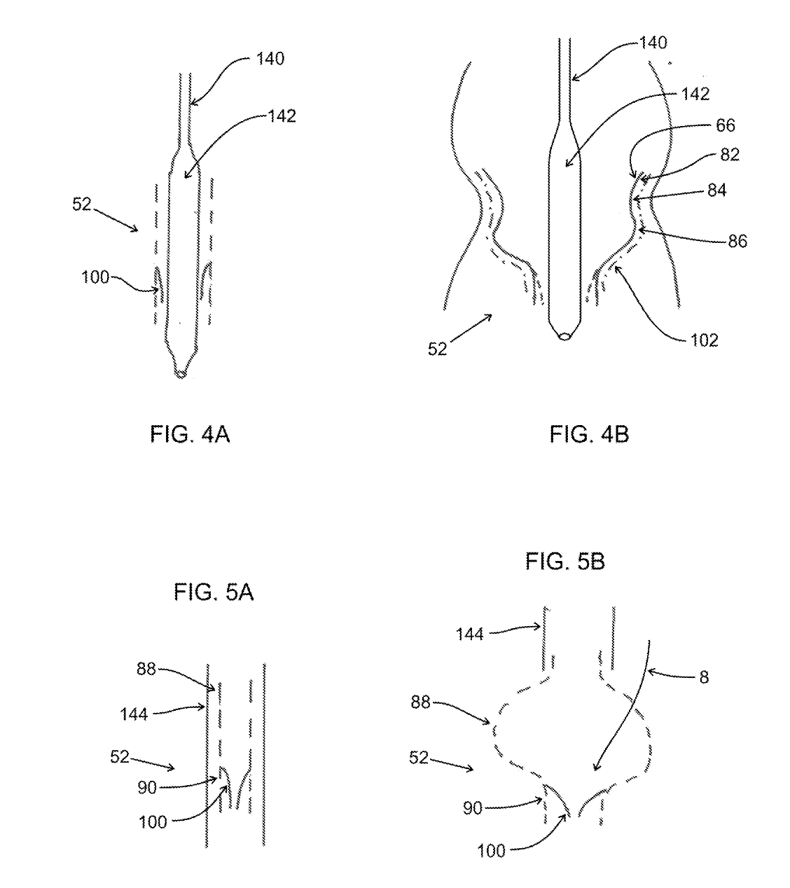

An alternate embodiment of the present invention is shown in FIG. 3; this embodiment has exchanged the SE tract portion stent of the previous embodiment for a BE tract portion stent. The BE tract stent can be formed of the same material as the BE stabilizing stent and can be contiguous with it. The delivery system for this embodiment is shown in FIG. 4A-4B. FIG. 4A illustrates a delivery configuration, in which replacement valve 52 comprising replacement leaflets 100 is affixed onto the outside of delivery balloon 142 located at the end of delivery catheter 140. Delivery catheter 140 comprises balloon 142, which is a dilation balloon as is known in the art, such as a cylindrical balloon used for dilating vessels, chambers, or openings in the body, or for expanding and deploying stents into body vessels. Upon reaching the appropriate site across the mitral valve, balloon 142 is inflated to place the BE stent 66 into position adjacent the mitral annulus 20. Waist 84 is again sized to be slightly smaller than mitral annulus 20. Upper bulb 82 and lower bulb 86 are expanded outwards to a larger diameter than mitral annulus 20. FIG. 4B illustrates the deployed configuration of stent 66 and replacement valve 52, after balloon 142 has been deflated.

In its deployed configuration, the BE tract element 90 has a tapered region 92 and a cylindrical region 94 where replacement leaflets 100 are attached as described earlier. Covering 102 is placed and attached to the outside of the tract element 90 and also to the stabilizing portion 88 to prevent perivalvular leaks. The tract diameter 98 of the cylindrical region 94 is small enough to not make contact with left ventricular lateral wall 32 and small enough to not push the anterior native valve leaflet 22 outwards into the left ventricular outflow tract 34. The cylindrical region 94 of the tract element 90 has a diameter in the range of about from 15-30 mm.

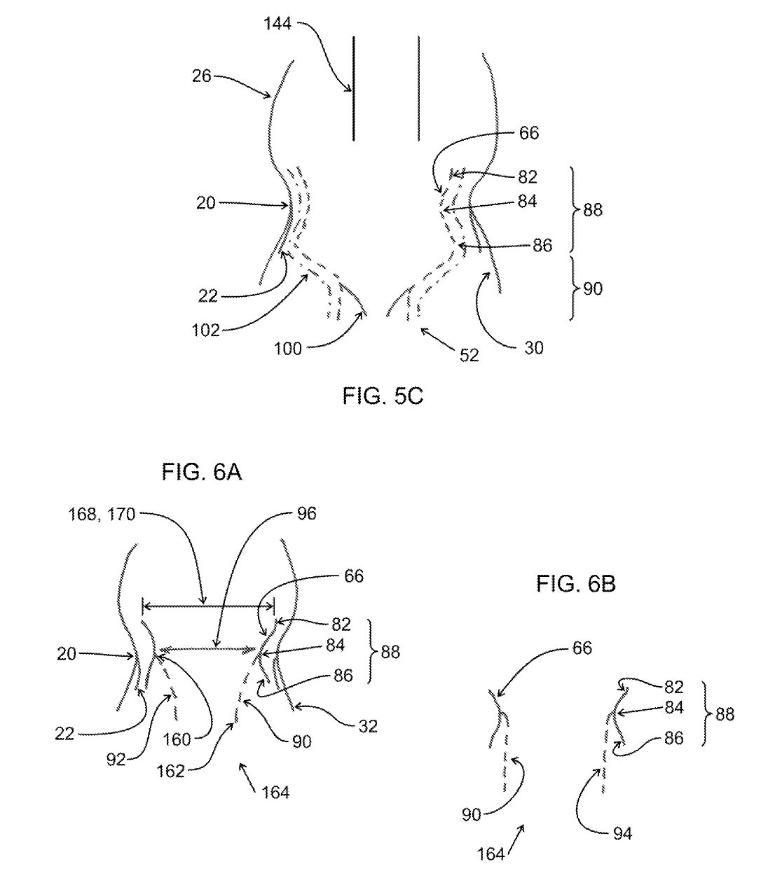

Another embodiment of the present invention has a stabilizing portion 88 and a tract element 90 formed from a SE material as shown in FIGS. 5A-5C. The stabilizing portion 88 is formed via thermal forming of NiTi or other elastic metals such that waist 84 has a diameter that is smaller than mitral annulus 20 so that waist 84 does not exert continuous outward forces that could cause further expansion of mitral annulus 20. Upper bulb 82 and lower bulb 86 are formed such that they have larger diameter than waist 84 and mitral annulus 20. The stent structure, stent angles, or expansion limiters described in the embodiments of FIGS. 1-4 can be used to form the smaller waist 84. Reference numerals found on this figure are similar to those used in previous drawings (FIGS. 1-4).

The delivery of the embodiment shown in FIG. 5 is shown in FIG. 5A-5C. Replacement valve 52 is held via sheath 144 in its non-deployed configuration during its delivery through the vasculature to the site of implant. Upon removal of sheath 144, replacement valve 52 expands outwards as shown in FIG. 5B. Stabilizing portion 88 and tract element 90 expand outwards to a smaller diameter to allow antegrade blood flow 8 to pass through replacement leaflets 100 while the proximal end of replacement valve 52 is still held by sheath 144. Upon release from sheath 144 as shown in FIG. 5C, stabilizing portion 88 expands outward towards mitral annulus 20 such that the upper bulb 82 and lower bulb 86 engage left atrial wall 26 and native valve leaflets 22 in the left ventricle to hold replacement valve 52 from migration. Sheath 144 can alternatively be configured such that a portion of sheath 144 is advanced downstream to allow stabilizing portion 88 of stent 66 to be deployed first and lock onto mitral annulus 20. Further distal movement of sheath 144 releases tract element 90 adjacent native valve leaflets 22.

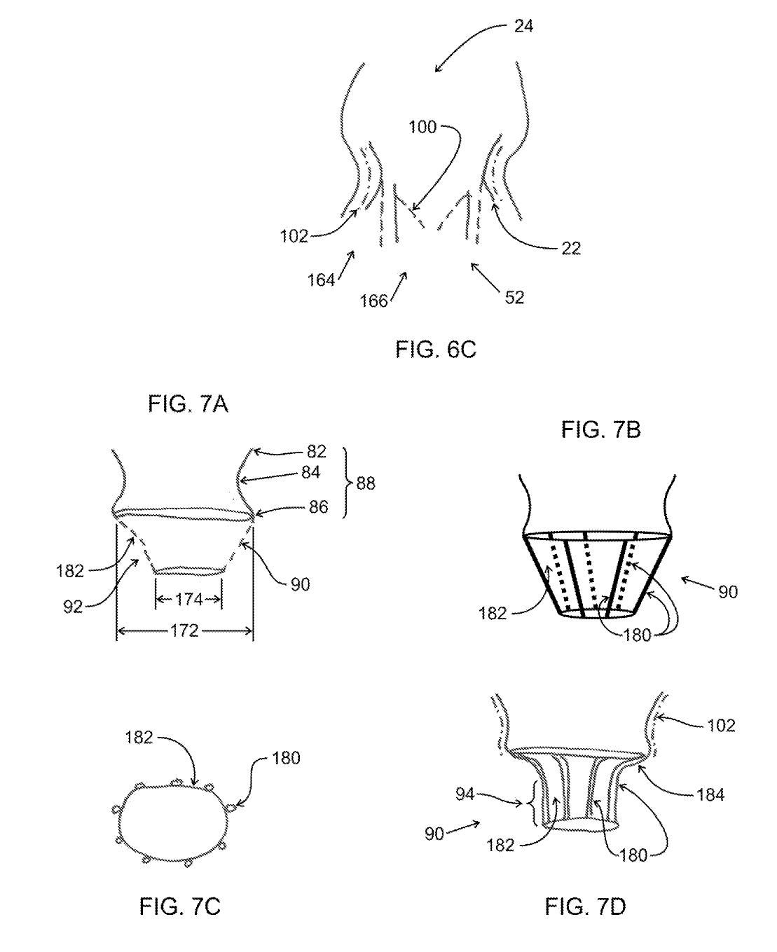

Another embodiment for the present invention is shown in FIGS. 6A-8E, this embodiment places the TMVR in two steps in order to provide a lower profile for the initially placed device containing the stabilizing stent and temporary valve leaflets during delivery and also provide a more accurate delivery adjacent the mitral annulus. In this embodiment, first component 164 is placed in position and deployed first, and second component 166 is placed in position and deployed second, where at least a portion of second component 166 is located within and secured to a portion of first component 164. FIGS. 6A-6C shows first component 164, which comprises stent 66 having a BE stabilizing portion 88 with a smaller diameter waist 84 and a larger diameter upper bulb 82 and larger diameter lower bulb 86. Attached to stabilizing portion 88 is tract element 90. Tract element 90 can comprise a conical or other tapered shape in tapered region 92 as shown in FIG. 6A. Alternatively, tract element 90 can comprise a cylindrical region 94 as shown in FIG. 6B. Tract element 90 can be attached to waist 84 of stabilizing portion 88 or to the upper bulb 82 or lower bulb 86 of the stabilizing portion 88. First component 164, comprising stent 66 with stabilizing portion 88, and tract element 90 are implanted in the first step. Waist diameter 96 is smaller than upper bulb diameter 168 and lower bulb diameter 170. Upper bulb diameter 168 and lower bulb diameter 170 can be the same, as illustrated in FIG. 6A, or they can be different diameters. Tract element 90 extends between the native valve leaflets 22. Tapered region 92 with a tapered region outlet 162 significantly (i.e., at least 20%) smaller than tapered region inlet 160 and a gradually enlarged diameter further upstream towards the tapered region inlet 160 and mitral annulus 20 provides added space between the tract element 90 and left ventricle lateral wall 32 to ensure that blood flow occurs in that space and that blood stagnation does not occur in that area. Also, the conical shape of tapered region 92 of tract element 90 allows hemodynamic forces due to blood pressure and blood flow to push the native valve leaflets 22 against the outside wall of the tract element 90 to reduce the space between the native valve leaflets 22 and the tract element 90 and reduce the potential for blood stagnation that can lead to thrombo-emboli and the potential for stroke. Tract element 90 can also have both a cylindrical region 94 and tapered region 92, where tapered region 92 comprises a flange or funnel at the inlet end of tract element 90 as shown in FIG. 7D, for example. Tract element 90 provides for the function of a temporary valve and provides the strength and support for implantation of second component 166 therein, as shown in FIG. 6C. Second component 166 comprises replacement valve 52, where the replacement valve 52 comprises a SE (or BE) structure having replacement leaflets 100 attached to its inner surface and is placed within tract element 90 as shown in FIG. 6C. Tract element 90 can provide for temporary valve function by containing temporary leaflets, or it can provide temporary valve function by being formed from fabric 182 as shown in FIG. 7A, which folds or collapses inward to form a valve when hemodynamic pressures would cause retrograde flow through tract element 90. At least a portion of tract element 90 can comprise axial fibers 180 as shown in FIG. 7B-7D which help to maintain integrity of the structure yet allow fabric 182 of tract element 90 to collapse to function as a temporary valve. A replacement valve 52 such as a SE or BE TAVR device or other stent-valve having a smaller diameter (at least 20% smaller) than the mitral annulus 20 can be implanted into tract element 90 of the present invention to form a functioning mitral valve implant. Implantation of replacement valve 52 within tract element 90 is performed as the second step. Covering 102, such as a skirt or other layer or outer covering such that blood cannot leak around the tract stent thereby forming a perivalvular leak, is attached to tract element 90.

Covering 102 of the present embodiment shown in FIGS. 6A-7D can be contiguous with tract element 90. Covering 102 can expand outwards to a larger diameter as the stabilizing portion 88 is enlarged to a larger diameter. Tract element 90 can be fabric material such as Dacron, for example, that achieves a specific diameter that is significantly smaller than that of the mitral valve annulus and retains that diameter when exposed to outward forces such as those imposed by replacement valve 52 or other secondarily implanted stent-graft implanted into tract element 90. In an alternate embodiment, tract element 90 can be expandable such that it can enlarge in the radial direction, for example, when a stent-valve is implanted within its lumen or against its inner surface. Such an expandable housing can be formed from polyurethane, silicone, spandex, or other materials having an elastic character, or by fabric or fibrous materials having fiber orientation allowing such expansion. Tract element 90 of this embodiment can expand into contact with the stabilizing portion 88 which can restrict diameter increase for tract element 90 and hold tract element 90 to a diameter that is significantly smaller (i.e., at least 20% smaller) than the diameter of the mitral annulus 20 and significantly smaller than upper bulb 82 and lower bulb 86 of stabilizing portion 88.

Stabilizing portion 88 can have a waist diameter 96 that is similar to the diameter of the mitral annulus 20 which typically has an average diameter of 31 mm (range 27-35 mm) in an adult but could be of a larger diameter (greater than 40 mm) in patients having an enlarged or dilated heart. Upper bulb 82 and lower bulb 86 of the stabilizing portion 88 are 1-8 mm larger than the diameter of waist diameter 96 or the diameter of the mitral annulus 20.

FIGS. 7A-7D show the structure for tract element 90 of the present embodiment. Tract element 90 as shown in FIGS. 7A and 7B is formed from fabric 182 having a conical shape extending from tapered region inlet 160 having a tapered region inlet diameter 172 which matches the diameter of the stabilizing portion 88 to which it is attached, to a smaller diameter at tapered region outlet 162 having a tapered region outlet diameter 174 which is smaller than tapered region inlet diameter 172 but still large enough to deliver blood from the LA to the LV, approximately 15-25 mm effective diameter. Tract element 90 can attach to the downstream end of lower bulb 86 as shown or to waist 84 of stabilizing portion 88. The conical shape provides a natural hemodynamic force against the outer surface of fabric 182 to close tract element 90 and prevent retrograde blood flow toward the LA during systole; tract element 90 itself can thereby provide the function of a temporary valve. The diameter of tract element 90 ranges from approximately 24 mm (range 19-29 mm) at its outlet end to 31 mm (range 27-35 mm for a normal-sized heart or over 40 mm for an enlarged heart) at its inlet or upstream end where it is attached to the stabilizing portion. Incorporated with tract element 90 is a series of 1-20 axial fibers 180 (preferably 4-16) spaced around the perimeter of fabric 182. Tract element 90 could have a single axial fiber 180 attached to fabric 182 and the single axial fiber 180 could function to prevent the housing from everting or migrating into the left atrium. Axial fibers 180 prevent fabric 182 from everting and extending into the LA due to hemodynamic forces created by the LV during systole.

Fabric 182 of tract element 90 in some embodiments can be formed from a weave, knit, or braid of a polymeric material such as PET or Dacron, for example. Alternately, fabric 182 can be formed from a sheet or tube of ePTFE or other polymeric sheet material. Further, fabric 182 can be a fibrous material formed from fiber spinning or a composite material with metal or polymeric fibers contained. Fabric 182 can be formed from a spandex material or other elastic fiber material, for example, that allows for expansion up to a set dimension and then no longer will expand and will provide friction to hold a stent-valve such as replacement valve 52 that is implanted within tract element 90. Such fiber is formed by placing a Dacron fiber wind around a polyurethane core, for example. Fabric 182 should be able to support the stress load created by blood pressure and should be flexible so that it can fold easily between the axial fibers 180. Axial fibers 180 should be of a strong enough modulus to resist bending that could cause eversion of the leaflets but not so high of a modulus to negatively affect flexibility for delivery via a catheter to the site of mitral replacement. A metal axial fiber formed from a nickel-titanium alloy, stainless steel, or monofilament PET or other higher modulus polymer monofilament strand ranging in diameter from 0.005-0.014 inches would serve to provide these characteristics, for example. Axial fiber(s) 180 are attached to fabric 182 via adhesive, thermal bonding, thermal sandwiching, sutures, weaving it into the fabric, or via other means known in the medical device industry. Axial fibers 180 can be metal, which are attached to stabilizing portion 88 via forming it contiguously with the stabilizing stent, or by brazing, welding, bonding, suturing, mechanically affixing, or other methods used to join metals. As shown in FIG. 7D, fabric 182 can have a flange 184 or funnel located on the end that is attached to the stabilizing portion 88. The flange allows tract element 90 to have a cylindrical region 94 that is significantly smaller in diameter (i.e., at least 20% smaller) than the mitral annulus 20 or waist diameter 96. Tract element 90 can range in diameter between approximately 15-25 mm, but may be larger at the tapered portion inlet or flange, to match the diameter of the portion of stabilizing portion 88 to which it is attached. Cylindrical region 94 provides a landing zone for the implantation of the replacement valve 52 or stent-valve containing the replacement leaflets 100.

Tract element 90 of the present invention can comprise a cylindrical, flanged, or conically shaped stent frame. The stent frame can have an open or closed cell wall structure and can be formed, for example, from ringlets 122 having a zig zag structure, and the ringlets can be connected together, for example, by connectors. Alternately, the stent wall structure for tract element 90 can similar to that of any known stent structure used in coronary or peripheral vascular stenting or used in current TAVR devices. The stent wall structure can, for example, be a SE stent formed from NiTi or other elastic metal including stainless steel alloys. The stent used for tract element 90 can be contiguous with stabilizing portion 88 of stent 66.

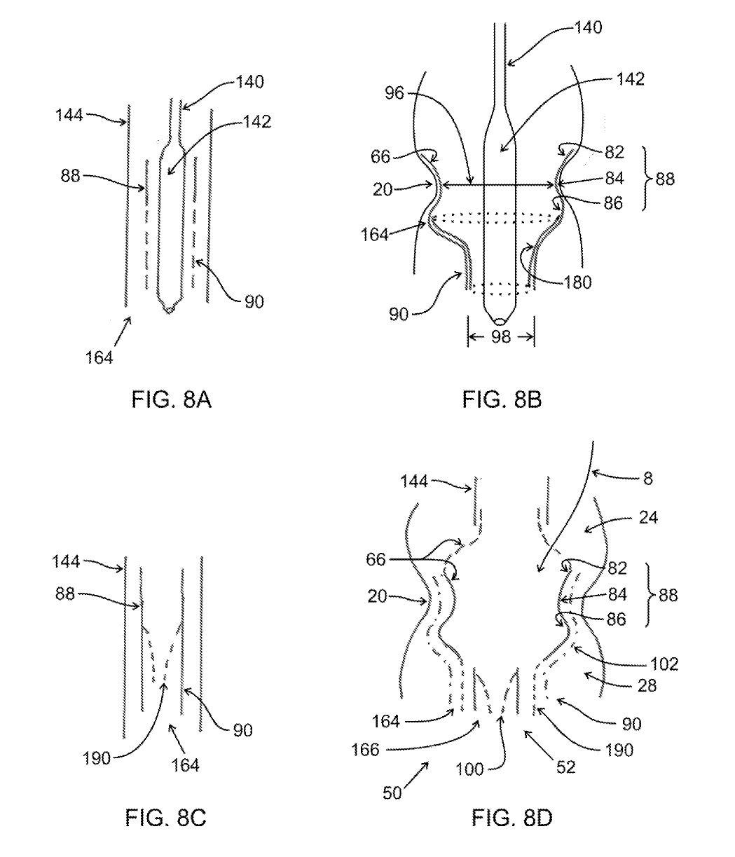

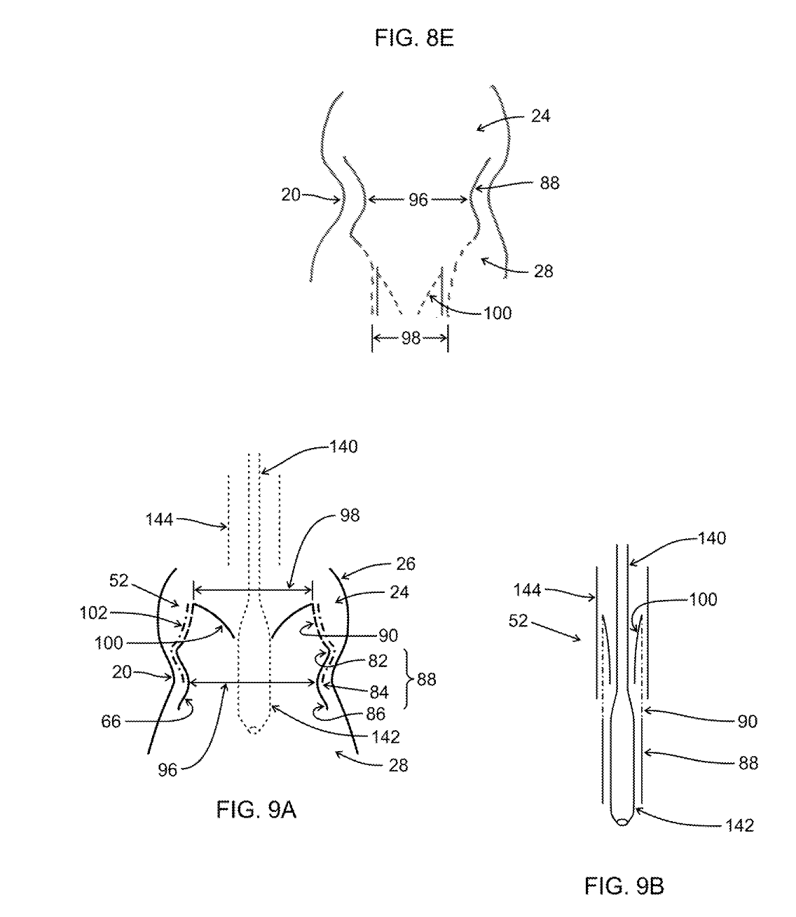

A two-step embodiment of the present invention can comprise replacement valve 52 as a TMVR device delivered as a second component 166 after mounting a first component 164 comprising a BE stabilizing portion 88 onto the outside of balloon 142 at the end of delivery catheter 140 as shown in FIG. 8A. Sheath 144 holds the tract element 90, which can include a SE stent structure, in a smaller diameter configuration for delivery. Upon retraction of sheath 144, any SE structures of tract element 90 expand, and fabric 182 can move to function a temporary valve supported by axial fiber(s) 180. After retraction of sheath 144, balloon 142, such as those cylindrical dilation balloons, for example, that are known in the art, is inflated to expand the BE stabilizing portion 88 adjacent mitral annulus 20 with upper bulb 82 and lower bulb 86 expanding to a diameter larger than mitral annulus 20 and the waist 84 slightly smaller than mitral annulus 20 as described earlier. Tract element 90 can also comprise a BE stent structure, in which case balloon 142 is longer so that tract element 90 also expands outward. Whether tract element 90 comprises SE stent elements, BE stent elements, or a combination, tract element 90 expands to a smaller tract diameter 98 than the waist diameter 96 or the diameter of upper and lower bulbs 82 and 86 as shown in FIG. 8B. A shorter length balloon that extends only through the stabilizing portion 88 is also anticipated.

FIGS. 8C and 8D show an alternate embodiment of the device similar to that illustrated in FIGS. 8A and 8B except that the stabilizing portion 88 is now formed from a SE material. Tract element 90 can have temporary leaflets 190 attached to the tract element 90 as shown; tract element 90 can be formed as previously described including axial fibers 180. The delivery of the device through the vasculature in the non-deployed configuration is accomplished by placing sheath 144 over the stabilizing portion 88 of stent 66 and tract element 90 as shown in FIG. 8C. Upon reaching the site of implantation, the sheath 144 is retracted and stabilizing portion 88 is expanded into contact, with the with upper bulb 82 in the left atrium 24 and the lower bulb in the left ventricle 28 against the native mitral valve leaflets 22. The waist 84 of the stabilizing portion 88 is adjacent mitral annulus 20 but does not exert outward force against the mitral annulus that could lead to possible expansion of the mitral annulus. A proximal portion of stent 66 can extend into sheath 144 as shown in FIG. 8D to allow repositioning or removal of the replacement valve 52 if not satisfied with its position; this proximal stent portion may be non-covered to allow antegrade blood flow 8 through this portion; alternatively, covering 102 can extend along the surface of stent 66. Prior to release of the stabilizing portion 88 and tract element 90, tract element 90 will function as a temporary valve awaiting the approval of the physician regarding its position adjacent the annulus. After the stent 66, stabilizing portion 88, and tract element 90 are released from sheath 144, the second component 166 of the replacement valve apparatus 50 for TMVR will be brought into the position indicated in FIG. 8D. The second component 166 of replacement valve apparatus 50, comprises replacement valve 52, which can be a stent-valve similar to a TMVR device, for example, with a stent structure and replacement leaflets. A sheath 144 (which can be the same sheath as in FIG. 8C, or a similar sheath) holds the replacement valve 52, which in this example comprises a SE stent structure and replacement leaflets 100, in a smaller non-expanded configuration. Retraction of sheath 144 allows the SE stent structure of replacement valve 52 to expand into the tract element 90 of first component 164 as shown in FIG. 8E. Alternatively, a replacement valve 52 which comprises a BE stent structure and replacement leaflets 100 can be placed into tract element 90 of first component 164.

Tract diameter 98 (the diameter of tract element 90) is significantly smaller than waist diameter 96 (i.e., at least 25% smaller); tract element 90 has a fixed diameter that can support the implantation of a stent-valve or TAVR device such as replacement valve 52 within the lumen of tract element 90 without significant expansion of tract element 90 and without leakage of blood or migration of the replacement valve 52. A covering 102 is attached along at least a portion of the surface of the stabilizing portion 88 and attached to tract element 90 to form a fluid-tight seal to prevent leakage of blood from the left ventricle 28 to the left atrium 24 when replacement valve 52 is closed. In an alternate embodiment, replacement valve 52 is a stent-valve with a stent structure which is expandable in the radial direction thereby allowing the implanted replacement valve 52 to stretch fabric 182 or other material of tract element 90 in a radial direction. In this alternate embodiment, the tract element 90 will resist expansion of the replacement valve 52 to a diameter that is at least 20% less than the diameter of upper bulb 82 or lower bulb 84.

FIGS. 9A-9B show a further embodiment of the present invention. The embodiment is similar to that described in FIG. 1 except that the tract element 90 is located upstream of the stabilizing portion 88 and is located in the left atrium. The stabilization portion of stent 66 is a BE stent such that it does not exert any continued excessive force against the annulus as could be found with some SE stents. The BE stabilizing portion has a smaller waist 84 positioned adjacent the annulus and a larger diameter upper bulb 82 located in the left atrium 24 and a larger diameter lower bulb 86 located in the left ventricle 28 as described in earlier embodiments. A SE tract element 90 is attached to stabilizing portion 88 and extends upstream into the left atrium. Located within tract element 90 and attached to its inner surface are replacement leaflets 100. In some embodiments, stent 66 comprises stabilizing portion 88 and tract element 90 and a single structure, such as a stent formed as a single piece of metal. In other embodiments stent 66 comprises stabilizing portion 88 but tract element 90 is a separate structure which has been attached to stent 66; this is one way to provide materials and structures with differing properties, such as a balloon expandable stabilizing portion and a self expandable tract element, for example.

The replacement valve 52 can be delivered as shown in FIG. 9B by mounting the BE stabilizing portion 88 onto balloon 142 of delivery catheter 140 as shown in FIG. 9B. Sheath 144 located proximal to balloon 142 can hold the SE tract element 90 in a non-expanded configuration for delivery through the vasculature to the mitral site. Expansion of balloon 142 deploys waist 84 of stabilizing portion 88 adjacent mitral annulus 20 and the upper bulb 82 in the left atrium 24 and lower bulb 84 in the left ventricle 28. Once satisfied with the position of replacement valve 52, the sheath 144 is retracted to allow tract element 90 to expand in the left atrium 24 as shown in FIG. 9A. Tract diameter 98 is smaller than the waist diameter 96 to ensure that tract element 90 does not contact or abrade the left atrial wall 26.

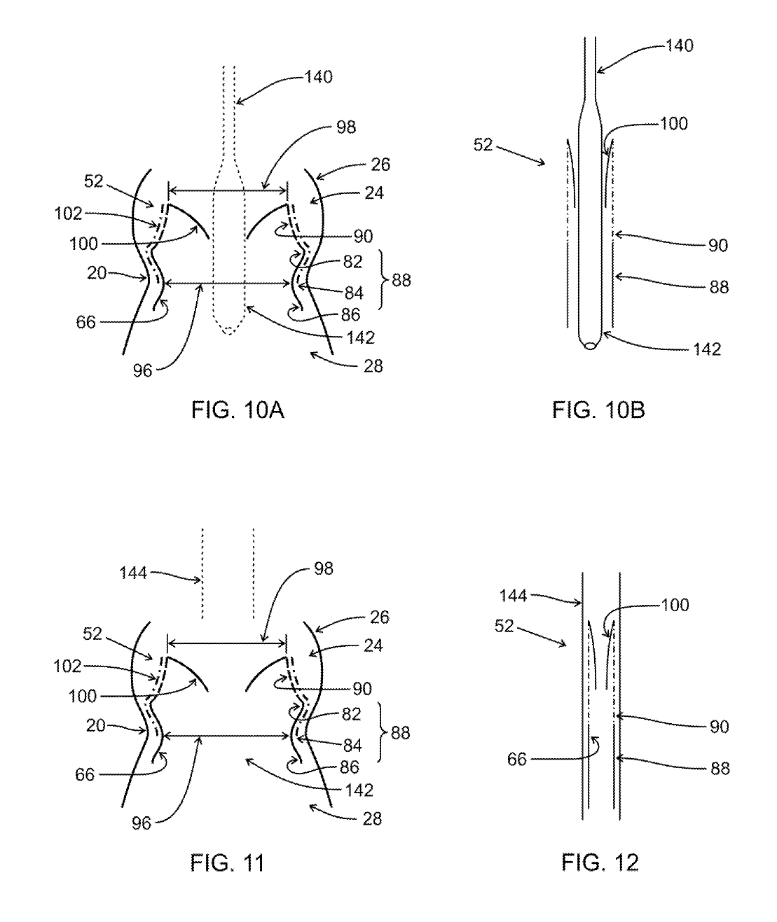

FIG. 10A-10B shows an embodiment similar to that of FIGS. 9A-9B except that the both the stabilization portion 88 and tract element 90 are formed from a BE material. The replacement valve 52 is delivered by affixing it to the outside of balloon 142 located on the end of delivery catheter 140. The waist 84 of stabilization portion 88 is positioned adjacent to mitral annulus 20 and balloon 142 is inflated. The waist 84 of stabilizing portion 88 is smaller than the mitral annulus 20 as described earlier. Upper bulb 82 and lower bulb 86 are larger in diameter than waist 84 and extend into the left atrium 24 and left ventricle 28 respectively to prevent migration of the device. Tract element 90 of stent 66 has covering 102 attached to it to prevent leakage of blood around the replacement leaflets 100. Tract element 90 of stent 66 is curved to ensure that the upstream edge of tract element 90 does not contact or abrade the wall of the left atrium.

FIGS. 11-12 show an embodiment similar to that of FIGS. 9A-9B except that both the stabilization portion 88 and tract element 90 are formed from a SE material. Forming the entire stent out of a SE material allows several advantages. The profile of the device in its non-expanded configuration as illustrated in FIG. 12 is smaller due to the presence of only sheath 144 to hold stent 66 in a non-expanded configuration and the absence of a balloon. Additionally, the device can be withdrawn back into the external sheath if the physician does not like its position.

As in other embodiments, the stabilizing portion 88 has a smaller diameter waist 84 and a larger diameter upper bulb 82 and lower bulb 86 as. The two bulbs hold the stent 66 in position across the mitral annulus 20 without the waist 84 making contact with the annulus or pushing the annulus outwards significantly. The tract element 90 containing replacement leaflets 100 is located in the left atrium 24 upstream of the stabilizing portion 88 and is contiguous with the stabilizing portion. The tract diameter 98 is smaller than the waist diameter 96; tract diameter 98 can be significantly smaller than waist diameter 96 (i.e., at least 25% smaller) to prevent contact of the upstream edge of the stent 66 with the left atrial wall 26.

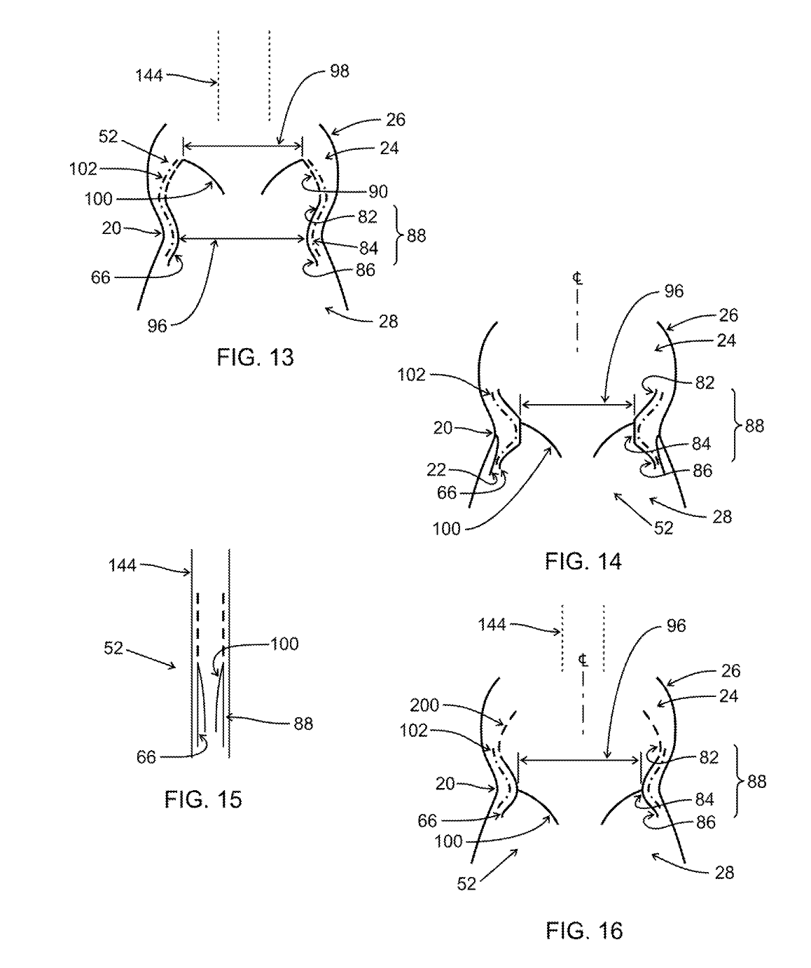

Replacement valve 52 is loaded into sheath 144 as shown in FIG. 12 for delivery through the vasculature. Initial retraction of sheath 144 allows the stabilizing portion 88 to make contact with the tissue of the LA and LV above and below the annulus and the waist 84 to be positioned adjacent the annulus. At this point, repositioning or removal of the device can be accomplished by advancing sheath 144 again if the positioning, sizing, or other aspects are not optimal. Further retraction of sheath 144 allows the tract element to expand and be released into the left atrium as shown in FIG. 11. Tract element 90 can be straight, curved outward as depicted in FIG. 11, or curved inward as shown in FIG. 13; the curved shape of FIG. 13 allows tract element 90 to follow a rounded shape of the left atrium and thereby to avoid sharp contact of the end of stent 66 with the wall of the left atrium thereby avoiding abrasion or potential arrhythmias.

An additional embodiment for the device is similar to that shown in FIGS. 9A-9B except that stabilizing portion 88 of stent 66 is a SE material and the tract element 90 is a BE material. The delivery of this device to the mitral site requires both a balloon for expansion of the BE portion and an external sheath for release of the SE portion. In this embodiment, the BE tract element 90 is affixed to the balloon and is not expanded until the position of the SE stabilizing portion 88 adjacent mitral annulus 20 is acceptable to the physician.

In many embodiments of the invention described herein, shown in the tract diameter 98 of the tract element 90 is significantly smaller (i.e., at least 25% smaller) in diameter than the waist diameter 96 or the effective annulus diameter. This significantly smaller tract diameter allows the tract element of the stent to avoid contact with the LA wall thereby avoiding abrasion of the LA wall and also provides a smaller profile to the TMVR replacement valve. A tract diameter of approximately 26 mm (range 20-31 mm) will provide adequate blood flow from the LA to the LV under most circumstances.

Yet another embodiment of the present invention is shown in FIG. 14. In this embodiment a BE stabilizing stent has a waist that is placed adjacent the annulus with a diameter significantly (i.e., at least 20% smaller diameter) smaller than the annulus. At each end of the waist are located an upper bulb and a lower bulb; the upper bulb has a larger diameter than the waist and is positioned in the LA. The upstream edge of the upper bulb can curve toward the centerline of the mitral valve to avoid contact of the upstream edge of the stent with the wall of the LA. The upper bulb can expand into the LA by 5-15 mm to assist in axial alignment of the stabilizing stent with the axis of the mitral valve annulus. The lower bulb extends into the LV for approx. 2-10 mm in the axial direction and has a larger diameter than the waist. The upper and lower bulbs have axial lengths ranging from 4-10 mm. The waist has an axial length of approx. 3-15 mm and has a generally cylindrical shape. In an alternate embodiment, the stabilizing stent can be formed with a diameter that applies direct contact with the mitral annulus to provide additional attachment force for alignment and for preventing migration of the stent.

In this embodiment, the replacement leaflets are located within the waist and are attached to the inner surface of the waist. A covering is located along the surface of the stabilizing stent to help reduce the likelihood for perivalvular leaks. In this embodiment, replacement leaflets 100 are attached to waist 84; therefore, this embodiment does not require a distinct tract element 90. Since the waist diameter 96 is smaller than the diameter of mitral annulus 20, this approach still allows smaller replacement leaflets 100 to be used, providing a smaller delivery profile. Since the waist diameter of the present embodiment is smaller than the effective annulus diameter, the area of valve leaflet material will be less than that of the native mitral valve leaflets; this reduction in area helps to reduce the profile of the device in its non-expanded configuration.

The BE TMVR is loaded onto a dilation balloon and positioned such that the waist of the TMVR stabilizing portion 88 is adjacent the mitral annulus 220. Upon inflation of the balloon, the waist of the stabilizing portion is located adjacent the annulus and the upper and lower bulbs of the stabilizing stent portion have been expanded outwards to a bulb diameter that is larger than the waist diameter. The upper bulb located in the LA prevents antegrade migration of the TMVR and the lower bulb in the LV prevent retrograde migration of the TMVR. The lower bulb also holds the native mitral valve leaflets outwards to make unrestricted space for the operation of the replacement leaflets.

In another embodiment, the stabilizing portion 88 is formed from a SE material as shown in FIGS. 15 and 16. In this embodiment, the waist 84 and upper bulb 82 and lower bulb 86 have similar diameters and functions as described for the BE stabilizing portions previously described. In this embodiment, the waist 84 is still smaller than the mitral annulus 20 thereby affording the benefit of smaller profile for the device in a non-expanded configuration (FIG. 15). In this embodiment, replacement leaflets 100 are attached to waist 84; therefore, this embodiment does not require a distinct tract element 90. Since the waist diameter 96 is smaller than the diameter of mitral annulus 20, this approach still allows smaller replacement leaflets 100 to be used, providing a smaller delivery profile.

Since the stent 66 used in this embodiment is totally SE, it can be held in a non-expanded configuration using only the sheath 144. Partial retraction of the sheath will allow positioning of the lower bulb 86 in the left ventricle 28, waist 84 at mitral annulus 20, and upper bulb 82 in the left atrium 24, so replacement valve 52 is placed across mitral annulus 20. Upper bulb 82 and lower bulb 86 each have a larger diameter than the waist 84. Upper bulb 82, which is located in the LA has a non-covered portion 200 located upstream of replacement leaflets 100 that may not require a covering to avoid retrograde blood leakage past replacement valve 52. At least part of non-covered portion 200 can be retained within sheath 144 until the position of replacement valve 52 is determined to be correct and the upper bulb 82 and lower bulb 86 are correctly positioned above and below the annulus. This allows sheath 144 to be advanced over replacement valve 52. withdrawing replacement valve 52 back into sheath 144, so that replacement valve 52 can be repositioned or removed if needed. Non-covered portion 200 can be released into the LA upon further retraction of the external sheath as shown in FIG. 16B when the position of replacement valve 52 is determined to be correct. Non-covered portion 200 can be curved toward the centerline of the mitral annulus such that the edge of the upper bulb does not contact or abrade the left atrial wall, which could lead to potential arrhythmias or damage to the left atrial wall. The lower bulb 86 has the requirement of preventing migration of replacement valve 52 in a retrograde direction toward the LA. The lower bulb 86 also is required to hold the native valve leaflets 22 outward and provide space for operation of the replacement leaflets 100. The lower bulb 86 cannot push the anterior leaflet outward too far as to restrict blood flow through the LVOT.

In an alternate embodiment shown in FIGS. 17A-17B, a TMVR valve similar to those shown in FIGS. 15-16 can be formed from a stent 66 with a stabilizing portion 88 that has a waist 84 formed from a BE material and an upper bulb 82 and lower bulb 86 formed from a SE material. The dimensions of waist 84, upper bulb 82, and lower bulb 86 are as described for FIGS. 15-16. The replacement leaflets 100 are contained within and attached to waist 84. The delivery system comprises delivery catheter 140 with balloon 142 as well as sheath 144 as shown in FIG. 17A. Stabilizing portion 88 is affixed onto balloon 142 for delivery. Upon partial retraction of sheath 144, the SE lower bulb 86 expands outwards making contact with the native mitral leaflets in the LV. Further retraction of sheath 144 past waist 84 allows waist 84 to be dilated by balloon 144. Full retraction of sheath 144 allows SE upper bulb 82 to expand into contact with the LA just above mitral annulus 20. Free end 80 at the upstream edge of upper bulb 82 can be curved toward the centerline of the mitral annulus to ensure that abrasion of the left atrial wall 26 does not occur as shown in FIG. 17B.

In embodiments of the invention shown in FIGS. 14-17B waist 84 is significantly smaller (i.e., at least 25% smaller) in diameter than the effective diameter of mitral annulus 20 (i.e., the diameter of a circle with the same perimeter as the annulus). This significantly smaller waist diameter provides a smaller profile to the TMVR valve. The waist diameter of these embodiments can range from 20 mm-31 mm and will provide adequate blood flow from the LA to the LV for normal heart function during sedentary or low-activity conditions.

The balloon described herein for delivery and deployment of the various stents and can be a dilation balloon known in the art for dilating or separating tissues of the body, such as a vessel, tube, or opening. The balloon can have a small diameter for introduction and can expand to a larger diameter when inflated, as is known in the art. Such balloons can be cylindrical in shape, and can be constructed of standard materials known in the art, such as PET, nylon, pebax, polyurethane, PVC, polyolefin, and others.

Transcatheter aortic valve replacement (TAVR) devices are being used for treatment of aortic valve disease, but the larger mitral valve does not yet have a viable transcatheter replacement. Current TAVR devices cannot be used in the mitral position; reasons for this include the larger size of the mitral annulus, and differences in anchoring requirements due to differences in adjacent tissues. The current TAVR devices could not be adapted for the mitral position simply by re-sizing the TAVR devices. First, a TAVR-type device sized large enough to fit a mitral annulus, especially in function mitral regurgitation (a common problem requiring mitral valve treatment, in which the annulus is dilated to an abnormally large size), because the resulting device would be too large for practical delivery via catheter. In addition, any advancement of disease that resulted in further dilation of the mitral annulus could result in dislodgement of the replacement “TMVR” valve device. Further, the existing TAVR devices anchor in place at the aortic valve by expansion of balloon-expandable (BE) or self-expanding (SE) stents which exert force on adjacent tissues to hold the TAVR device in position; such expansile forces could cause the already oversized mitral annulus to dilate even further, which is undesirable. For these and other reasons, prior TAVR devices are not used for treating mitral valve dysfunction. In addition to the various replacement valve embodiments described herein, the present invention provides an adapter that allows implantation of currently available TAVR devices for treatment of mitral valve dysfunction. Stent-valves with diameters that are less than those of typical mitral valve annuli can also be implanted to treat mitral valve dysfunction by use of the present invention. It is anticipated that the present adapter will allow TAVR devices (or other stent valves) that are typically 10-70% smaller in diameter than a mitral valve annulus to be used to provide transcatheter mitral valve replacements, TMVR; in a vast majority of cases the diameter of the aortic annulus (and aortic stent-valve) is at least 20% smaller than the mitral annulus and is often 30-50% smaller. In one embodiment the adapter provides a housing or tract element for a stent-valve having a diameter 15-25% smaller than the mitral valve annulus; such stent-valves, like those used for TAVR procedures, can range from 21-29 mm in diameter. The adapter in another embodiment can provide a housing for a stent valve that is 10-50% smaller than the diameter of the mitral valve annulus. One advantage of such an adapter is that current TAVR devices (or similar sized stent-valve devices) have a significantly lower profile while being delivered to the treatment site than a large diameter stent-valve sized to fit the larger diameter of the mitral valve annulus. In addition, the smaller aortic sized (i.e., approximately 21-29 mm diameter) stent-valve devices are less likely to impact upon the anterior mitral valve leaflet to interfere with the LVOT, and also less likely to impact upon the LV lateral wall during LV contraction.

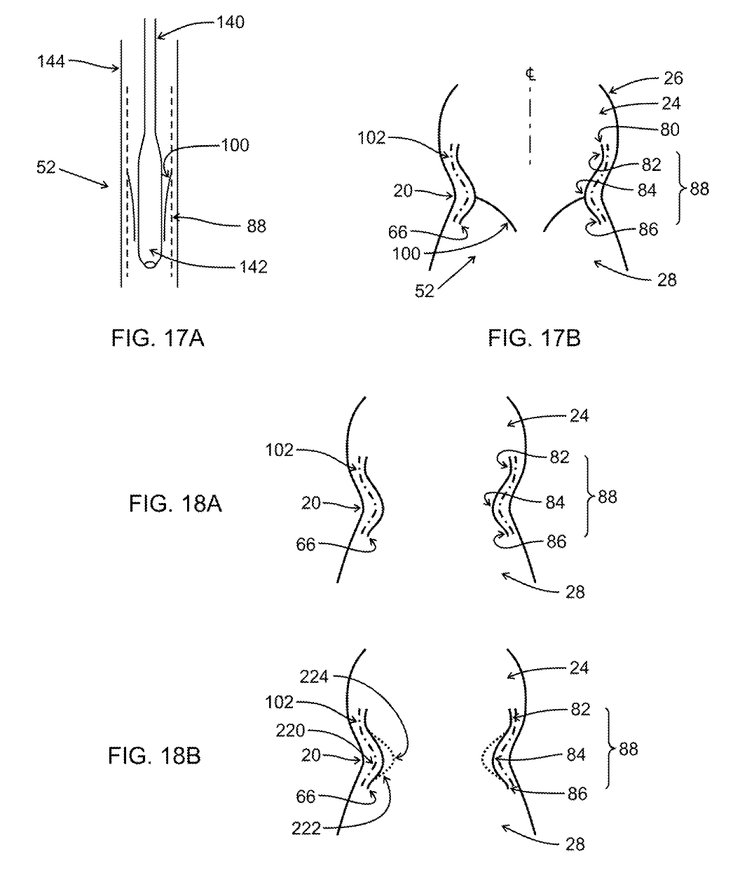

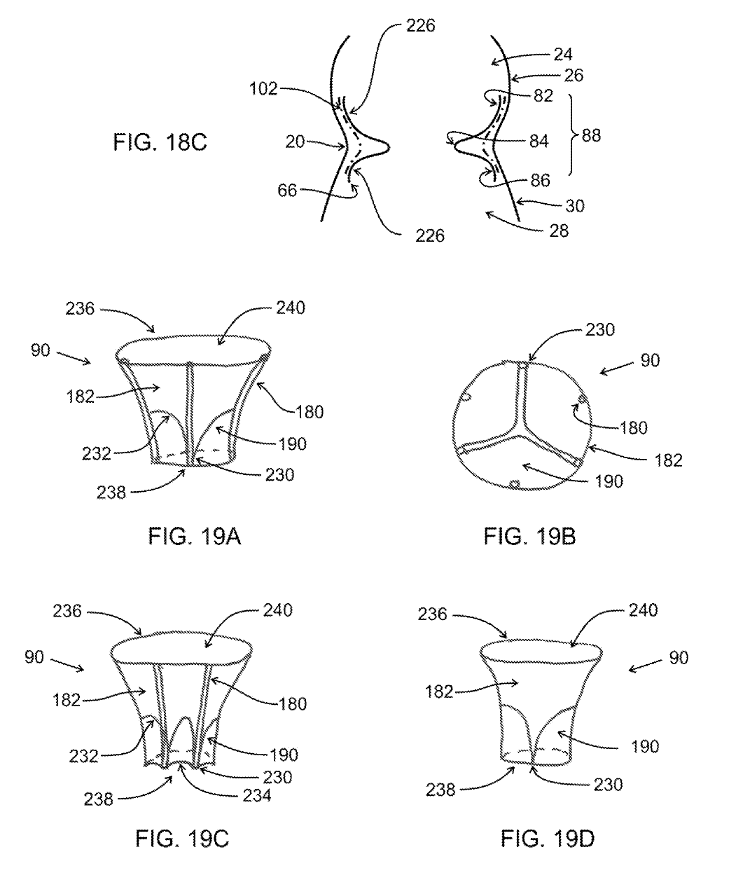

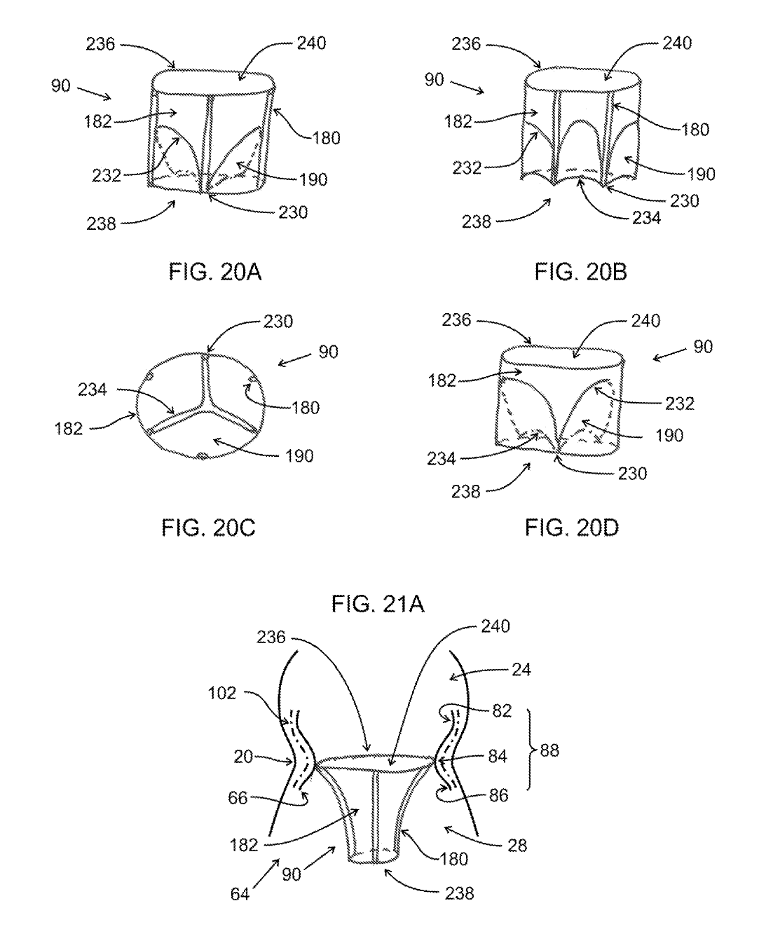

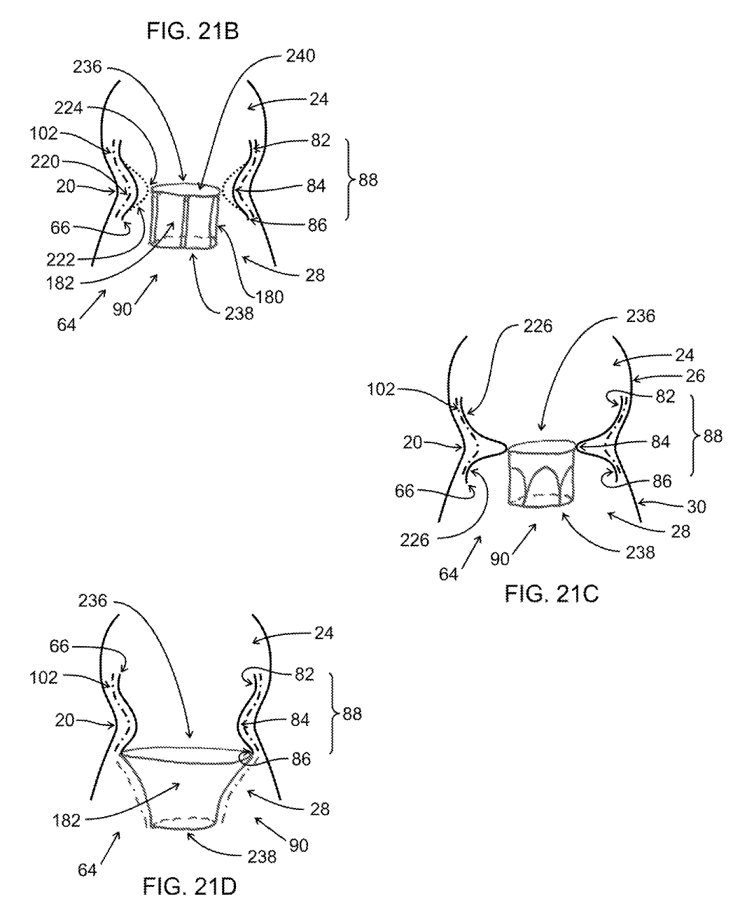

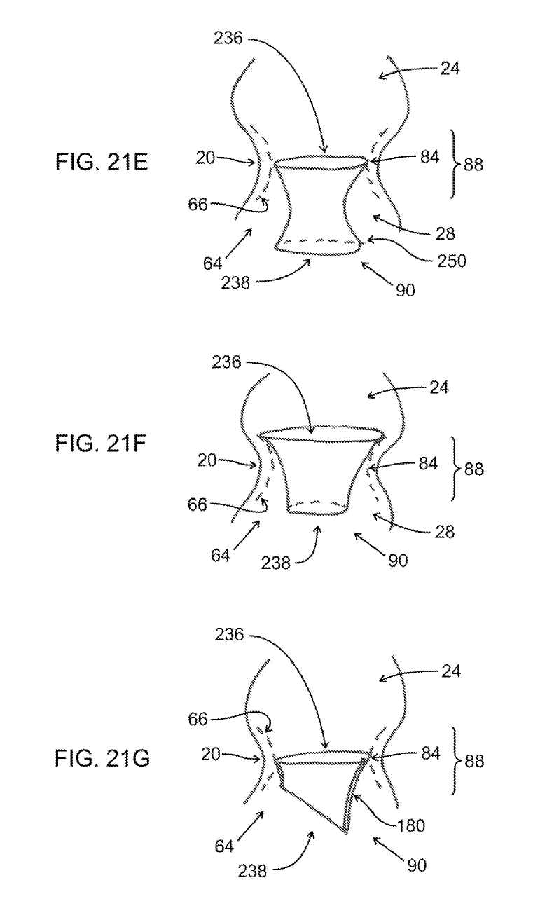

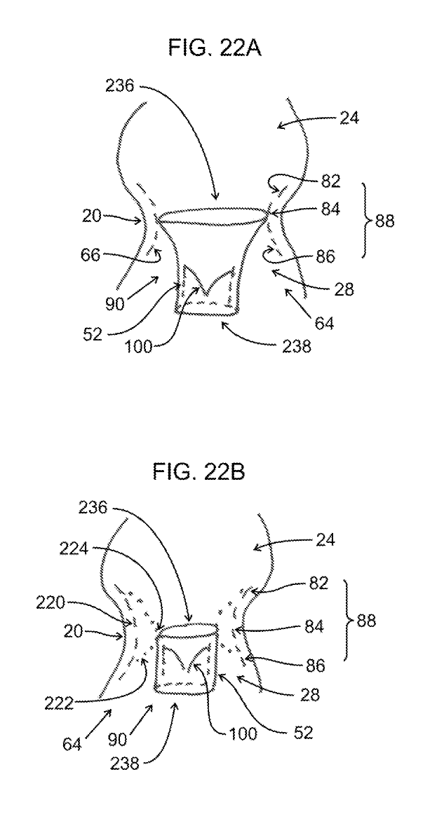

The adapter of the present invention comprises a stabilizing stent as shown in FIGS. 18A-18C and a housing or tract element as shown in FIGS. 19A-20C; the two components are attached together as shown in FIGS. 21 and 22. The stabilizing stent or stabilizing portion, and the housing or tract element, can be formed as a single contiguous structure with metal struts or filaments that form portions of both components, or a contiguous structure which functions as both a stabilizing portion and a tract element, or they can be formed as separate structures and then attached or bonded together.

The stabilizing portion 88 can have a structure that is the same as that described in FIGS. 6A-6C and elsewhere herein, and further in FIG. 18A. Stabilizing portion 88 can be a stent, and may be described herein as the stabilizing stent. Stent 66 as described elsewhere herein, can be the stabilizing stent, incorporating stabilizing portion 88 as described herein. In one embodiment the stabilizing portion 88 has a narrow waist 84 that is placed adjacent to mitral annulus 20 or the material of the native valve leaflets 22 (shown in other figures herein) that are attached directly to the mitral valve annulus. Stabilizing portion 88 also comprises upper bulb 82 located in the left atrium 24 and lower bulb 86 located in the left ventricle 28, both bulbs having a larger diameter than the waist 84 which is located adjacent the mitral valve annulus. The lower bulb 86 pushes outwards against the anterior and posterior mitral valve leaflets to provide anchoring for the stabilizing stent against migration into the left atrium. The upper bulb 82 expands outwards within the LA to a diameter larger than the mitral annulus to prevent migration of the stabilizing stent into the LV and to provide a seal with the tissues of the LA wall and the mitral annulus to prevent leakage of blood around the outside of the stabilizing portion 88. The waist 84 forms a seal with the mitral annulus or native leaflet tissues to prevent leakage of blood around the adapter, between the adapter and the mitral valve and LA tissues. The stabilizing portion 88 can be formed from either a BE material or a SE material known in the stent art, and expanded outwards via a balloon, or a mechanical expansion means, or via self-expansion after release from a constraining sheath. All or part of the stabilizing portion 88 is covered with a covering 102 to prevent blood flow through the stent and to prevent blood flow around the replacement valve located within the stent or housing; the covering can be formed from a polymeric mesh, weave, microporous material, or other material commonly used as a covering for covered stents. Covering 102 can be located on the inside and/or outside surface(s) of at least a part of stent 66, or otherwise incorporated with stent 66. The stabilizing portion 88 of this embodiment shown in FIG. 18A has a waist diameter that is approximately equal to (or approximately 1-3 mm smaller than) the diameter of the mitral valve annulus which ranges from 27-35 mm in most patients and can be over 40 mm for patients with enlarged left ventricles. The upper and lower bulbs can be at least 1-8 mm larger than the waist diameter.

In another embodiment, as shown in FIG. 18B, the stabilizing portion 88 has an outer stent structure 220 and an inner stent structure 222; outer stent structure 220 has an upper bulb 82 located in the LA that is expanded to a diameter that is larger than the mitral annulus 20. The waist 84 of the outer stent structure 220 makes contact with the tissues of the mitral annulus or the native mitral valve leaflets to provide friction or interference that will help prevent migration and also to form a fluid tight seal against the tissues. The lower bulb 86 expands outwards in the LV to a diameter larger than the mitral annulus 20. The stabilizing portion 88 also has an inner stent structure 222 that is either attached to or contiguous with the outer stent structure 220, which narrows to an inner waist 224 which is at least 20% smaller than the diameter of the mitral annulus 20. Inner structure 222 provides an attachment site for the housing that is described later. The waist 84 of outer stent structure 220 has a diameter approximately equal to the diameter of the mitral valve annulus having an average diameter of 31 mm. The inner waist 224 of inner stent structure 222 is at least 20% smaller than the mitral valve annulus and approximately 20% smaller in diameter than waist 84 of outer stent structure 220 with a diameter of approximately 26 mm (range 20 mm-31 mm).

In yet another embodiment the stabilizing portion 88 can be formed with one contiguous component that forms upper bulb 82 and lower bulb 86 as well as forming a central region or waist 84 that has a smaller diameter that is at least 20% smaller than the diameter of the mitral valve annulus (range 10-35% smaller) as shown in FIG. 18C. The diameter of the waist 84 of this stabilizing stent embodiment is approximately 24 mm (range 19-29 mm). The upper and lower bulbs, as described earlier, have diameters that are larger than the mitral valve annulus and larger than the diameter of the waist (bulb diameter ranges from 3-8 mm larger than the waist diameter) to form a seal and to prevent migration of the stabilizing stent. At least a portion of upper bulb 82 contacts the left atrial wall 26, and at least a portion of lower bulb 86 contacts the left ventricular wall 30 and/or native valve leaflets 22 (shown in other figures) at tissue contact portions 226 which serves to anchor stent 66 in position at the annulus. A covering 102 is attached or joined to the stabilizing portion 88 to prevent leakage of blood between the stabilizing stent and the mitral valve annulus.

The adapter of the present invention can have a housing or tract element 90 such as shown in the embodiments of FIGS. 6B-7D. The housing shown in FIG. 7D has a flange or funnel located at its inlet end and a cylindrical portion that joins to the flange as previously described. Tract element 90 for the adapter is not required to contain temporary valve leaflets but can instead function as a valve as described earlier in reference to FIGS. 6A-8E. As shown in FIGS. 19A-C the housing of the adapter can have temporary leaflets 190 contained within its central lumen 240, functioning as a temporary valve. The temporary valve can be a trileaflet valve similar in structure to an aortic valve; the commissures 230 can meet at three of the axial fibers 180 to provide support for the three temporary leaflets 190. Temporary leaflets 190 can be attached to the tract element 90 along a crown-shaped attachment line 232. The leaflet free edges 234 of temporary leaflets 190 can be located near the tract element outlet end 238. A bileaflet valve similar in shape to those found in the native venous system can also be used for a temporary valve. As shown in FIG. 19D, the housing or tract element 90 can be formed without the axial fibers if the housing contains temporary leaflets 190 and has a frame structure provided by a stent-like frame to provide structural support; alternatively, the housing can comprise a more flexible fabric material with axial fiber(s) 180 for structural support plus temporary leaflets 190. A further alternative is that the housing or tract element 90 can be formed from a fabric 182 and axial fiber 180 structure that does not contain temporary leaflets, as described above; in this case the fabric material can move to function as temporary leaflets as described above. The temporary leaflets 190 can be formed from a polymeric material such as polyurethane, ePTFE, PET, or other thin polymeric or woven fabric material that can withstand the forces of left ventricular pressure that can exceed 150 mm Hg. The temporary leaflets 190 are preferably thinner than a standard tissue leaflet to provide a reduced profile during delivery; a wall thickness ranging from 0.0005-0.003 inches is adequate for most polymeric films to provide such strength.

The housing or tract element of this embodiment can have a flange such as that described earlier in reference to FIG. 7D, with a diameter at the inlet end that is approximately 31 mm or more, or a diameter that is similar to that of the mitral valve annulus. This flange diameter should match the diameter of the waist of the stabilizing stent to which it is attached. The diameter of the cylindrical portion of the tract element should be approximately 24 mm (range 19-29 mm) to approximately equal the diameter of the replacement valve 52 (TAVR device or other stent-valve, for example) that is to be implanted within the tract element. The replacement valve 52 is held within the tract element by frictional forces as the stent-valve is expanded into contact with the housing. In one embodiment, tract element 90 has a cylindrical portion with a fixed diameter that can support the outward forces imposed upon it by an implanted TAVR device within the central lumen 240. The tract element will maintain a diameter that is at least 20% smaller than the diameter of the mitral annulus. Such a housing or tract element is comprises a material and structure that is nondistendable, such as woven PET, for example.

In other embodiments, tract element 90 can have comprise a tapered structure as described earlier. Various combinations of the illustrated cylindrical, tapered, and flange shapes for tract element 90 are anticipated.

In an alternate embodiment, the housing or tract element 90 comprises an elastic material such that it is expandable. Upon placement and expansion of a stent-valve within the central lumen of the housing, the housing is able to expand in a radial direction. Radial expansion of the tract element of some embodiments can place the tract element into contact with the stabilizing portion 88 to form a tight frictional fit that resists migration of the stent-valve. The expandable housing can be formed from an elastic material such as polyurethane, silicone, spandex, or other elastic polymer. Spandex provides the character that it can expand a prescribed amount in the radial direction and thereafter it becomes nondistendable.

Temporary valves comprising temporary leaflets found in some embodiments of the present invention can be formed from noncompliant material such a PET, Nylon, ePTFE, or other materials used in medical devices to form strong and thin fibers or films, including biological tissue materials. Such leaflets can be separated at their commissures or at the coaptation of the leaflet free edges to allow expansion of the temporary leaflets in a radial direction to form a larger opening; alternatively, the temporary leaflets can either stretch or alter their coaptation with neighboring leaflets to allow expansion of the temporary valve. Further alternatively, the curved free edges of the leaflets can provide for expansion of the perimeter of the valve. Temporary leaflets can comprise elastic materials such as polyurethane, silicone, thermoplastic elastomers, or composite. The temporary leaflets of the present invention are able to expand outwards from the diameter at deployment to a larger diameter without fracture and without generating excessive radial forces when expanded to a larger diameter by implantation of a stent-graft within the central lumen formed of the tract element.

The adapter can have a housing that is formed with a cylindrical shape such as that shown in FIG. 6B or in FIGS. 20A-20C. As described earlier in FIG. 6A-6C the structure of the housing with the axial fibers allows it to function as a temporary mitral valve. As shown in FIG. 20A a cylindrical housing can contain temporary valve leaflets. The leaflets can form a trileaflet valve or a bileaflet valve as described in FIGS. 19A-19D. While 3 leaflets and 2 leaflets are preferred, other numbers of leaflets can be used, such as 1 or 4 leaflets, to form a functioning temporary valve. The attachment of the temporary leaflets to the housing and the construction of the leaflets is as described for FIGS. 19A-19D. The free edges of the temporary leaflets are located near the outlet end of the housing. The cylindrical housing can contain temporary leaflets without the presence of axial fibers as shown in FIG. 20D. The attachment line 232 is preferably a crown-shaped curve similar to the attachment line of native trileaflet valves such as the aortic valve.

The diameter of the cylindrical housing or tract element of this embodiment is approximately 26 mm (range 20 mm-31 mm) and is approximately equal to the diameter of the TAVR device to be implanted within the housing. The diameter of the housing is approximately equal to the diameter of waist 84 (or diameter of inner waist 224 for the embodiment of FIG. 18B, for example) described earlier. The housing of the adapter can be formed with similar shapes, for example, conical or cylindrical, to the description found in FIGS. 19A-21G. The structure of the housing can be that of a SE stent. The SE stent can have a metallic wall structure of any stent used as a SE stent for vascular support or used in frames of current TAVR devices.

Embodiments of the adapter 64 of the present invention are shown in FIGS. 21A-21G. In FIG. 21A a tract element 90 having a flange is attached to waist 84 of stabilizing portion 88. The larger diameter flange has a large diameter that is similar to the diameter of the mitral annulus 20 and similar to the diameter of the waist 84 of the stabilizing portion 88. The attachment of the housing or tract element 90 to the stent 66 is made via methods described in FIGS. 6A-8D. The attachment can be made by adhesive, thermal bonding, brazing, welding, suturing extending common components from the stabilizing stent to the housing, or other methods. The stabilizing portion 88 and tract element 90 are attached together prior to implantation and are implanted as a single adapter unit. In this embodiment the housing or tract element 90 does not contain temporary leaflets, but instead the housing itself acts to form the function of a temporary mitral valve or mitral valve leaflet system. The housing or tract element 90 can collapse at tract element outlet end 238 along its perimeter as one portion of the housing makes contact with another portion adjacent to it to form a seal and act as a temporary valve; the axial fiber(s) 180 prevent the tract element 90 from everting into the left atrium 24 due to pressure generated by left ventricular contraction. Alternatively, this embodiment can have temporary leaflets 190 contained within the tract element 90 as described in FIGS. 19A-19C.

The embodiment of FIG. 21B shows adapter 64 having a cylindrical housing or tract element 90 having the tract element inlet end 236 attached to the inner waist 224 of the inner stent structure 222. The diameter of the housing is significantly smaller (i.e. at least 20% less) than the diameter of the mitral valve annulus (range 10-35%). This embodiment utilizes tract element 90 to provide temporary valve function without having temporary leaflets attached to the inner surface of the tract element. It is understood that temporary leaflets could have been placed within tract element 90 as shown in FIGS. 20A-20C; alternatively, adapter 64 can comprise replacement leaflets 100 affixed to tract element 90 such that the adapter 64 is a one-step device that provides full mitral valve function upon its implantation.

The embodiment of FIG. 21C shows an adapter 64 having the inlet of a cylindrical tract element 90 attached to a central waist 84 of a stabilizing portion 88 that has a single contiguous structure that extends from upper bulb 82 to lower bulb 86 having diameters larger than the mitral annulus 20, with a central waist 84 having an inner surface that is smaller than the mitral annulus 20 by at least 20% (range 10-35%). In this embodiment, portions of tract element 90 function as temporary leaflets to provide a valvular function and allow only antegrade blood flow from tract element inlet end 236 to tract element outlet end 238. Alternatively, the tract element 90 can have temporary leaflet(s) 190 or permanent valve replacement leaflets 100 attached to the housing as previously described; such leaflets can be, for example, formed from tissue materials such as pericardial or valve tissue. Tract element 90 can comprise axial fibers 180, as previously described, to provide structural assistance in holding the commissures 230 of the temporary leaflets 190. Alternately, as shown in FIG. 6B, the tract element 90 can be formed with axial fibers 180 but without temporary leaflets.

A covering 102 can be attached to the fabric 182 of the housing or tract element 90 of various embodiments of the present invention described herein; covering 102 can attach along the surface of upper bulb 82 of the stabilizing portion 88 or along the entire surface of stent 66. The covering 102 is formed from a polymeric material, for example, that prevents the flow of blood or fluids through the fabric and prevents fluids from flowing around or bypassing the valve portion of the adapter or implanted valve. The tract element in some embodiments can be a fixed diameter housing that allows a stent-valve to be implanted against its inner surface and hold the stent-valve at a diameter that is significantly smaller than the diameter of the mitral valve annulus. In an alternate embodiment, the tract element can be expandable to form a larger diameter housing upon implantation of a stent-valve within its lumen or against its inner surface. The covering can be formed from materials that allow it to be either a fixed diameter or expandable in diameter.

The embodiment of FIG. 21D has the tract element inlet end 236 positioned at the distal end of lower bulb 86. In this embodiment, tract element 90 has a flared upstream end that can aid in holding lower bulb 86 in a larger diameter expanded configuration as illustrated. The tract element outlet end 238 has a smaller diameter (i.e., at least 20% less) than the diameter of the annulus. The tract element 90 can serve to provide a valvular function such as shown in FIG. 21A or it can serve to hold a set of temporary leaflets 190 near its downstream end; a smaller diameter stent-valve or TAVR device can be placed within the tract element to form a permanent valve function.

One embodiment of the adapter 64 of the present invention as shown in FIG. 21E has a tract element 90 that has an outlet end flair 250 or enlarged diameter at tract element outlet end 238. This flair can assist in holding a stent-valve implanted in tract element 90 to prevent it from migrating toward the left atrium due to forces generated by the left ventricle. A stent-valve that is implanted into this housing or tract element can have a similar flair to improve the frictional fit between the stent-valve and the tract element.

Another embodiment of the adapter 64 is shown in FIG. 21F having the tract element inlet end 236 attached to upper bulb 82 of stabilizing portion 88. This configuration allows the tract element 90 to extend to a lesser degree into the left ventricle and to a greater degree into the left atrium instead. The tract element 90 can be formed with a flange or taper as previously described and can have an outlet end flair 250 at tract element outlet end 238 if desired as shown in FIG. 21E.

In further another embodiment of the tract element 90 of adapter 64 as shown in FIG. 21G, a single axial fiber 180 can be attached to fabric 182 of the tract element 90 to hold it from everting. The tract element in this embodiment is intended to serve as a temporary valve while awaiting the implantation of a stent-valve into the tract element. It is understood that two, three, or more fibers can be attached to the housing to form one or more temporary leaflet(s), if desired.

Placement of a replacement valve 52 having replacement leaflets 100, in this case a BE TAVR device or a BE stent-valve device, into the tract element 90 is shown in FIGS. 22A and 22B. In FIG. 22A the BE stent-valve (replacement valve 52) is delivered to the cylindrical portion of tract element 90. It is expanded into place against the inner surface of the tract element 90 and is held there by frictional forces. The tract element 90 in this embodiment is formed from a PET fabric or other fabric that does not expand appreciably under the forces applied by the BE stent-valve (i.e., less than about 5%). As in other embodiments, a covering 102 on tract element 90 serves as a barrier or skirt to prevent leakage of blood through the stent walls of the BE stent-valve. The diameter of the BE stent-valve is matched to the diameter of the cylindrical portion of the tract element 90 and is at least 20% less (range 10-35%) than the diameter of the mitral annulus 20 or the diameter of the waist 84 or the upper bulb 82 or lower bulb 86 of the stabilizing portion 88. In FIG. 22B a BE stent-valve is placed within the cylindrical tract element 90 that is attached to inner waist 224 of inner stent structure 222.

The tract element of one embodiment can have a fixed diameter that is characterized by a housing material that is substantially nondistendable. In an alternate embodiment the tract element can be expandable such that it can expand in the radial dimension upon implantation of a stent-valve within it. The expansion force imposed by the tract element onto the stent-valve must be large enough to prevent migration of the stent-valve.

The embodiment shown in FIG. 22A shows the implanted stent-valve (replacement valve 52) located in the cylindrical portion of the tract element 90 that is located downstream of the mitral annulus 20. As shown in FIG. 22B, the implanted stent-valve can be positioned axially such that it is located within tract element 90 adjacent the wall of the stabilizing portion 88. Upon expansion of the stent-valve, the tract element 90 of one embodiment is able to expand outwards along with temporary leaflets 190 and covering 102 as previously described such that the stent-valve is able to apply radially directed outward force against the stabilizing portion 88 to generate friction to hold the stent-valve from migration and hold tract element 90 at a diameter that is at least 20% smaller than the diameter of the mitral annulus 90 or upper bulb 82 or lower bulb 86.

The present adapter 64 can utilize any of the stabilizing portions 88 with any of the tract elements 90 of the present invention described herein.

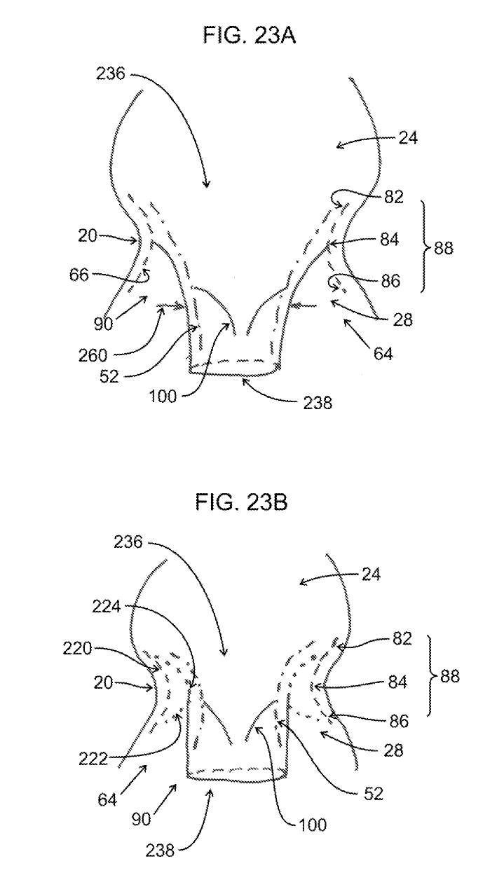

Placement of a replacement valve 52 having replacement leaflets 100, in this case a SE TAVR device or a SE stent-valve device into the tract element 90 is shown in FIGS. 23A and 23B. In FIG. 23A the portion of the SE stent-valve containing replacement leaflets 100 is delivered to the cylindrical portion of the tract element 90 and a portion of the stent-valve can extend across the mitral annulus 20 and can extend into the left atrium 24. The stent-valve is released to allow self-expansion into place against the inner surface of the tract element 90 and is held there by frictional forces. As in other embodiments, a covering 102 of the tract element 90 serves as a barrier or skirt to prevent leakage of blood through the stent walls of the stent-valve and thereby the covering prevents blood flow from bypassing the valve leaflets. The leaflet-containing portion diameter 260 of the stent-valve is matched to the diameter of the cylindrical portion of the tract element and is at least 20% less than the diameter of the mitral valve annulus (range 10-35%). In FIG. 23B a SE stent-valve is placed within the cylindrical tract element 90 that is attached to the inner waist 224 of the inner stent structure 222.

The present adapter 64 can utilize any of the stabilizing portions 88 with any of the tract elements 90 of the present invention described herein to provide an adapter to allow a stent-valve with a significantly smaller diameter than the mitral annulus to be held from migration and without leakage of blood from the LV to the LA.

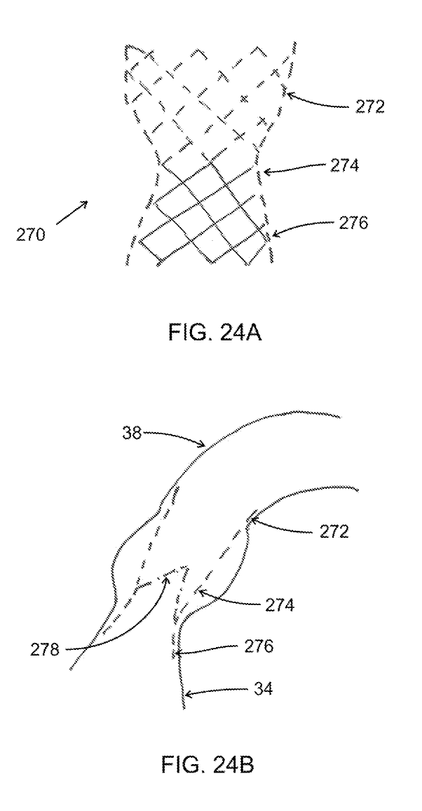

FIGS. 24A and 24B show an example of a SE TAVR device stent 270 having a closed cell structure used, for example, in a current SE TAVR device that is comprised of a connected closed-cell zig-zag structure. Aortic portion 272 of stent 270 placed in the aorta 38, sinus portion 274 is placed in the aortic sinus 44 and LVOT portion 276 is located in the left ventricular outflow tract 34 (LVOT). The LVOT portion 276 expands outward via an elastic expansion force to hold the stent tightly against the LVOT to prevent migration of the stent and to ensure that perivalvular leaks are minimized. Other portions of the stent similarly provide an outward force against native tissues to prevent migration and minimize perivalvular leaks. A covering along a portion of the stent that contains the TAVR device leaflets 278 to assist in preventing perivalvular leaks. The continued outward force supplied by LVOT portion 276 can place excessive forces onto underlying tissues such as nerves and can result in the development of heart block. If device such as the TAVR device just described were placed in the mitral position, a similar continued outward force would be applied to the mitral valve annulus via a SE stent; such continued outward forces could cause unwanted further annular dilation to the patient that has functional mitral regurgitation due to left ventricular dysfunction. The magnitude of this continual outwardly directed force by the stent is reduced by the present invention described below, which can provide zero outward forces in the fully expanded configuration of the stent.

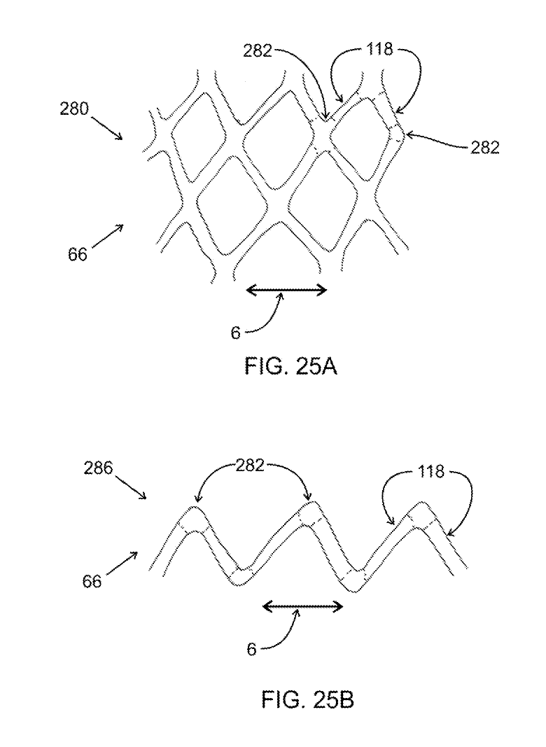

The wall structure of the stent 66 of the present invention can comprise a closed cell structure 280 as shown in FIG. 25A having hinge portions or hinges 282 that undergo much of the expansion deformation and stent struts 118 that connect between the hinges 282. The stent 66 can comprise a SE or BE stent material or structure. Stent 66 can comprise an open cell structure 286 such as the zig-zag structure that can be formed into a ring as partially shown in FIG. 25B; the zig-zag structure depicting the circumferential direction 6 of the ring can be connected to other neighboring zig-zag rings or the zig-zag structure can be non-connected to its neighboring ring. The zig-zag structure is comprised of hinges and struts that are connected in any pattern to form a wall structure for a stent or a stent-graft. Hinges 282 significantly deform as stent 66 is expanded from a small diameter configuration to a large diameter configuration. One hinge 282 is connected to another hinge 282 via a stent strut 118 do not significantly deform as stent 66 is expanded; the stent struts 118 of some embodiments can deform to a lesser degree during expansion deformation, but generally not as much as hinges 282.

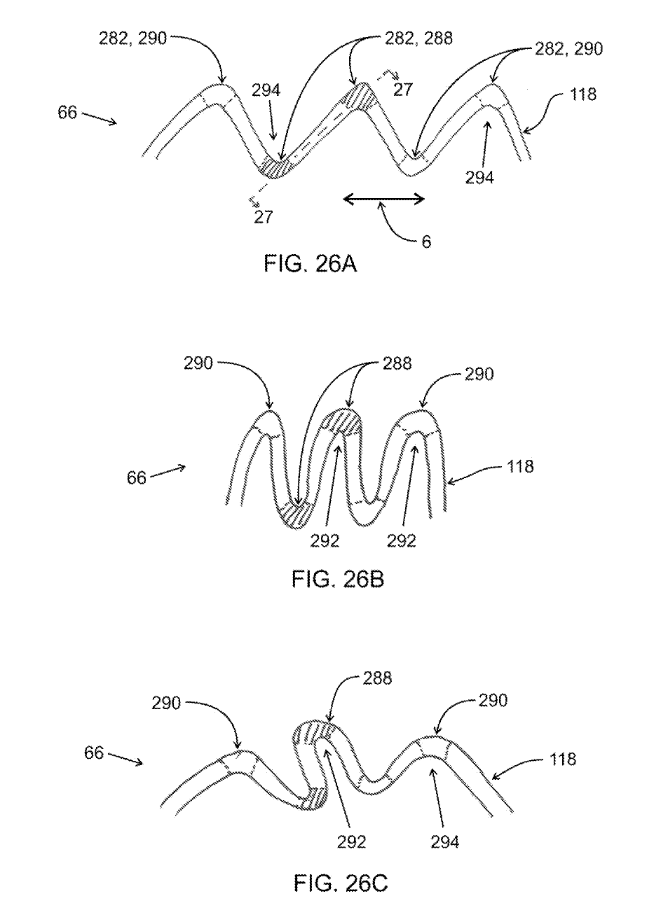

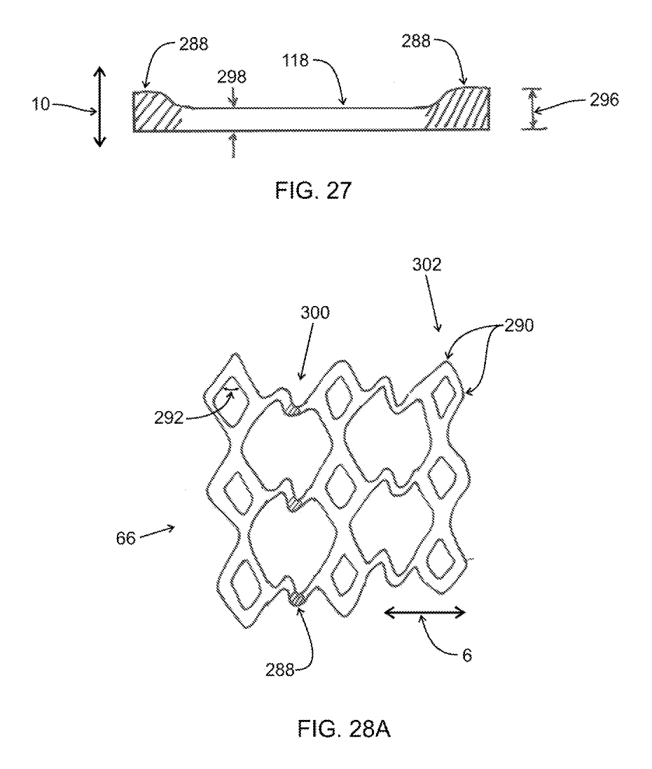

As shown in FIGS. 26A-26C is a combination BE/SE stent wall structure where at least some of the hinges 282 of stent 66 the present invention are BE hinges 288 formed from a BE material or plastically deformable material or material having a BE character that is determined by its shape, and other hinges 282 located in series with the BE hinges 288 are SE hinges 290 formed from a SE material, or elastically deformable material, or material having a SE character that is determined by its shape. Hinges 282 are connected to other hinges 282 via stent struts 118 that can be designed such that they do not undergo as much deformation as the SE hinges 290 during the expansion deformation of the stent. In some embodiments the stent struts 118 are designed to not undergo deformation during the expansion deformation. During compression of stent 66 into the sheath 144 for delivery as shown in FIG. 26B the BE hinges 288 and SE hinges 290 are in a small radius of curvature (RC) configuration 292. Upon release from sheath 144, the SE hinges 290 expand outwards to a large RC configuration 294 as shown in FIG. 26C, but the BE hinges 288 remain in the small RC configuration 292. The stent in this intermediate configuration can be further expanded outwards via a balloon dilation to form a larger configuration as shown in FIG. 26A, in which the BE hinges 288 have been expanded to a larger RC configuration 294.

FIG. 27 shows a cross sectional view of one embodiment of stent strut 118 and two BE hinges 288 as shown, for example, in FIG. 26A. The hinges 288 have a larger hinge height 296 in the radial direction 10 than the strut height 298. The larger hinge height 296 provides a greater strength to the BE hinge 288 so that it can resist bending deformation as stent 66 is compressed in sheath 144 for delivery, and resist bending deformation as stent 66 is released from sheath 144 for deployment. This larger hinge height 296 can be applied also to SE hinges 290.

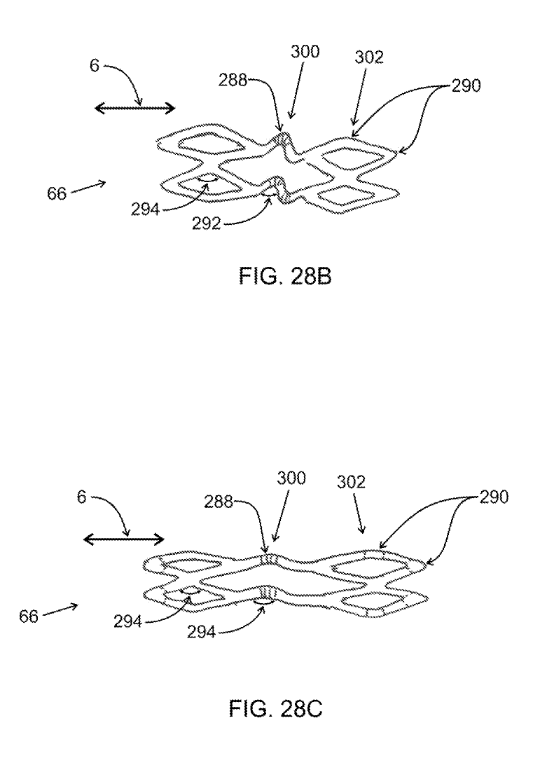

FIGS. 28A-28C show an example of a combination BE/SE wall structure for stent 66 of the present invention having a closed cell design. In this embodiment a SE portion 302 of stent 66 is in series with a BE portion 300 of stent 66. During compression of the stent during placement in a sheath 144, the SE portion 302 is compressed elastically to a small radius of curvature configuration 292 as shown in FIG. 28A; the BE portion 300 is also in its non-deployed configuration having a small RC configuration 292 in sheath 144 as shown in FIG. 28A. The BE portion 300 contains at least one BE hinge 288 that is delivered in a small RC configuration 292. Upon release of stent 66 from the sheath 144 as shown in FIG. 28B, the SE portion 302 of the stent expands outwards elastically in the circumferential direction 6 and makes contact with the vessel wall or other tissue at the implantation site, exerting a low outward force on the vessel wall or other tissue. At least some of the SE hinges 290 change configuration from a small RC configuration 292 to a large RC configuration 294 during the expansion deformation; the BE hinges 288 retain the small radius of curvature configuration 292. If additional outward force is required to attain improved contact of the stent with the vessel wall or other tissue, such as to reduce perivalvular leaks, the BE portion 300 can be further expanded in the circumferential direction 6 via balloon dilation as shown in FIG. 28C to form a large RC configuration 294 for the BE hinge 288.

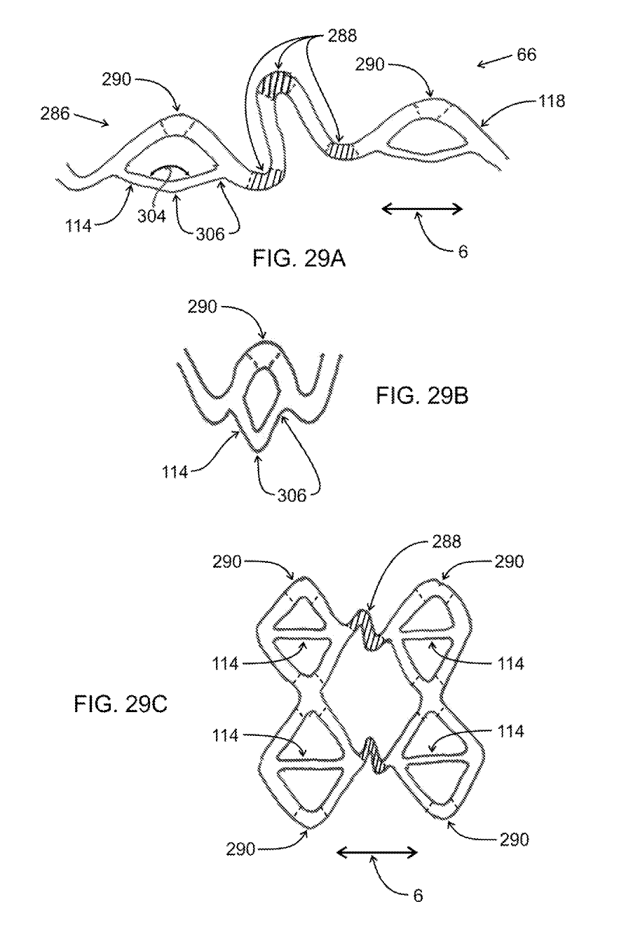

In another embodiment expansion limiters 114 are placed across the SE hinge 290 as shown in FIGS. 29A-29C. Expansion limiters 114 can be placed across the SE hinges 290 of the combination BE/SE stent wall structure described above or they can be placed across some or all of the SE hinges 190 of an entirely SE stent wall structure. The expansion limiter 114 is formed from a metal or polymeric material and is attached to the wall structure of the stent such as a stent strut 118 or hinge 282. The expansion limiter 114 is weaker than the stent strut 118 or hinge 282, such as by forming expansion limiter 114 with a weaker material, or by forming expansion limiter 114 with a smaller cross-sectional area than stent strut 118 or hinge 282. For example, expansion limiter 114 may be formed with a thickness and width ranging from approximately 0.002-0.005 inches, and smaller in diameter, width, and/or thickness than the SE hinges 290 or stent struts 118. Alternatively, expansion limiter 114 may be made weaker by other means (such as by thermal annealing, for example) than the stent struts 118, and with expansion limiter bend sites 306 that are weaker than the SE hinges 290. In order for expansion limiter 114 to easily deform with minimal force so as to not significantly affect the expansion forces of stent 66, expansion limiter 114 can have expansion limiter bend sites 306 can be located at the attachment of the expansion limiter 114 with the stent strut 118, for preferential bending deformation at the expansion limiter bend sites 306; alternatively, expansion limiter 114 can have bending deformation along the length of the expansion limiter 114. The expansion limiters 114 can be contiguous with the material of stent 66 and formed during the initial laser or machining process of the stent wall structure. During use, expansion limiter 114 is easily deformed at bend sites (see FIG. 29B) or throughout the length of the expansion limiter without the need for significant force in comparison to the outward force being exerted by the adjacent SE hinge 290 (which expansion is limited by expansion limiter 114). As stent 66 is released from sheath 144 and the SE hinge 290 is expanded outwards as shown in FIG. 29A or 29C, the expansion limiter 114 is placed into a configuration that is a relatively straight line with an expansion limiter angle 304 of approximately 160 to 180 degrees (ranging from about 140 to 180 degrees). In this way the expansion limiter 114 is placed into tension in the circumferential direction 6 and is able to resist further expansion of the SE hinge 290 due to tension forces on the expansion limiter 114. Expansion limiter 114 can be located in an open cell structure 286 such as that shown in FIG. 29A or a closed cell structure 290 as shown in FIG. 29C.

One method for forming the SE hinges 290 and BE hinges 288 of the present invention is by altering the dimensions of the hinges for the SE hinges 290 relative to the BE hinges 288. A stent wall structure that is laser or mechanically machined from a single metal can thus be formed into a stent of the present invention where some of the hinges are SE and others are BE.

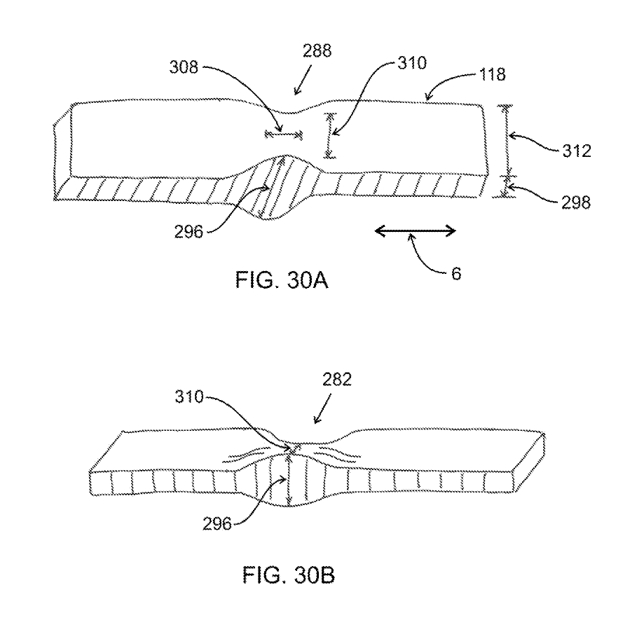

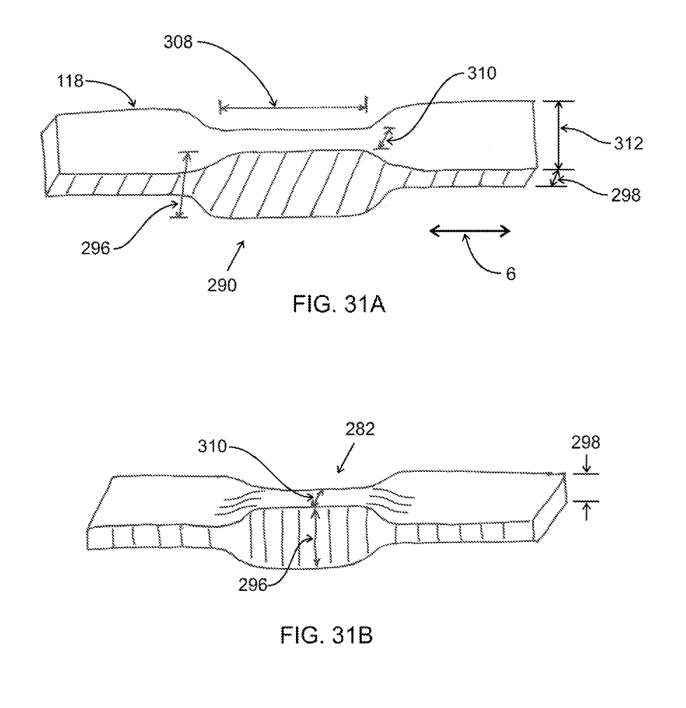

As shown in FIG. 30A a BE hinge can be machined such that it has a narrow hinge width that focuses the deformation over a small hinge length and thereby forces the metal to undergo plastic deformation in the hinge region during expansion deformation to a larger diameter or a larger RC. A long hinge length as shown in FIG. 31A will result in a SE hinge character or elastic character where the hinge will deform elastically during an expansion deformation to a larger RC.

FIGS. 30A and 30B show a top view and a side view of a BE hinge 288 that is formed primarily by machining and is not dependent upon thermal treatment to attain the characteristics of a BE hinge for either an elastic metal such as NiTi or for a less elastic material such as stainless steel. As shown in FIGS. 30A and 30B the hinge length 308 for a BE hinge must be short in comparison to the hinge length for a SE hinge (see FIGS. 31A and 31B for comparison) by at least a factor of two. The hinge height 296 (in the radial direction 10) is adjusted to provide the necessary outward force required by the hinge. For the BE hinge 288 the hinge height 296 is larger than the strut height 298 to provide the plastically deformable hinge with enough strength to resist bending during its compression within the sheath 144. In one embodiment the large hinge height is at least two times the strut height. The hinge width 310 of BE hinge 288 is less than the strut width 312 to provide the BE hinge 288 with the necessary weakness to concentrate the bending deformation during expansion of the stent 66 as the stent strut 118 does not bend significantly during the expansion deformation. Such a hinge design will provide plastic deformation character to an elastic metal such as NiTi. Other hinges 282 formed by an elastic metal that are not formed with such hinge dimensions can undergo standard SE hinge character. The BE hinge 288 can also be formed from stainless steel to form such BE hinge plastic deformation character.

FIGS. 31A and 31B show a hinge that has a SE character due to the long hinge length 308 and lack of focusing of the expansion deformation at hinge 282, which allows the metal of the hinge to retain its elastic character. As shown in FIGS. 8A and 8B the hinge length 308 for the SE hinge 290 is long in comparison to the hinge length 308 for the BE hinge 288 by at least factor of two such that during expansion deformation, the SE hinge 290 deforms elastically. The hinge height 296 in one embodiment is larger than the strut height 298; in this embodiment the outward elastic expansion force of the SE hinge 290 is enhanced and controlled to any desired level by increasing the hinge height 296. The SE hinge 290 can have a large hinge height 296 in the radial direction 10 to provide the magnitude of force necessary to hold the native valve leaflets 22 outwards in a TAVR stent application. The hinge width 310 of the SE hinge 290 is less than the strut width 312 such that the elastic deformation of the SE hinge 290 occurs without significant deformation of stent strut 118.

Thus a stent can be formed from BE hinges 288 formed from a BE hinge design as shown in FIGS. 30A and 30B and SE hinges 290 formed from a SE hinge design for other hinges as shown in FIGS. 31A and 31B. The material of construction can be an elastic metal or polymer or it can be a plastic metal or polymer; the design of the hinge determines whether it behaves as a BE or SE hinge.

An alternate embodiment of the present invention contains both SE hinges 290 and BE hinges 288 having a hinge height 296 that is similar to each other and also similar to the strut height 298 (not shown). This embodiment uses an alternate method for forming the SE hinges 290 and BE hinges 288 that requires thermal processing of metals such as NiTi, stainless steel, or other metals or polymers. Heat treatment of the NiTi or stainless steel is used to cause specific hinges or other component of the structure to be SE and adjacent parts to be BE; careful thermal shielding must be employed during the heating, quenching or rapid cooling operations.

One method of providing the required shielding is to fabricate a fixture that holds the stent in precise alignment such that the BE hinges 288, for example, are held at a specific location in space. A small tube (or other shaped fixture) of a highly thermally conductive material (including but not limited to copper or aluminum) runs in the specific locations to provide contact with the BE hinge 288 or BE component where BE response is desired. A hot fluid is pumped through the tubing, which prevents the immediate location of the BE hinge portion of the stent in contact with the tubing or other shaped fixture from being quenched, while the surrounding NiTi is quenched in a cold-water bath to provide the SE character to SE hinges or other components of the stent. In other methods, local heating such as with brief laser exposure at specific sites is used to provide local variations in heating, cooling, or quenching, to achieve the differing SE and BE properties in regions of stent 66. Masks and/or heat sinks can be utilized to help control the local heating and cooling.

The critical parameter that is controlled to provide BE character by the process for a NiTi alloy is the amount of time the NiTi soaks at the precise heat-treat temperature; the tubing or shaped fixture must have a very low thermal mass to permit the stent to reach the critical temperature as quickly and repeatably as possible. At the exact time that the fixture is quenched or plunged into ice water, the hot fluid is simultaneously circulated through the fixture to prevent the BE segments from being quenched along with the SE segments.

Another means of obtaining the desired SE/BE segments in a single stent includes heating small elements of the stent locally and sequentially with a laser, for example, thereby allowing the neighboring NiTi to quench the tiny locally heated zone. The BE segments would be created by allowing the laser to dwell at those locations of the BE hinge, for example, for a longer period of time.

Yet another means of obtaining the desired local formation of specific BE hinges is to heat-treat and quench the entire stent; then in a secondary operation, expose the desired BE segments or hinges with a local heating method (including but not limited to laser, hot probe, or induction) that allows the BE segments to cool slowly.

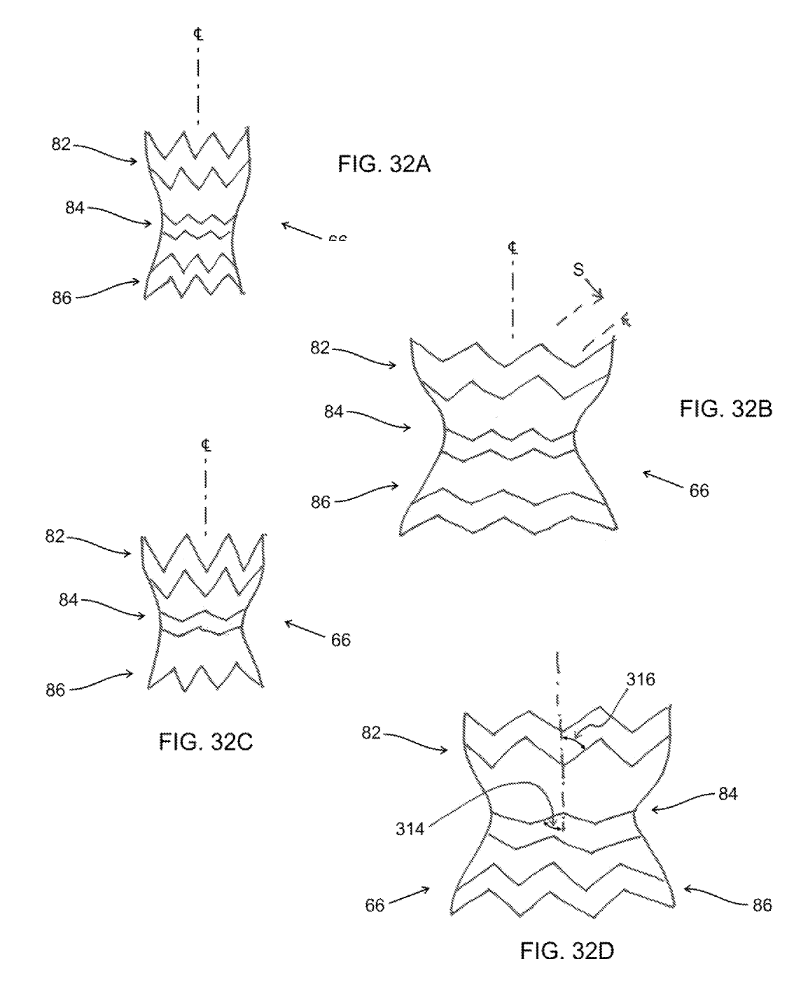

FIGS. 32A-32D show an embodiment for a stent 66 with a stabilizing portion 88 that can be used to stabilize a TMVR device or an adapter 64 of the present invention as described previously herein. The stabilizing stent can provide a housing or tract element 90 for temporary leaflets 190 such as those described in previous embodiments into which a second implant of a smaller diameter stent-valve can be placed; alternatively, a stent-valve having a stented housing and permanently attached leaflets can be attached to the stabilizing stent in a manner described earlier. The stabilizing stent is intended to attach to the tissues above (in the left atrium) and below (in the left atrium) the mitral annulus, and also attach or position itself in an axial direction adjacent to the mitral annulus. Some embodiments make full contact of the waist of the stabilizing portion of the stent with the mitral valve annulus; other embodiments are adjacent but not required to touch the mitral annulus along its entire perimeter. The stabilizing stent of one embodiment is formed from a series of zig zag rings or ringlets 122 that are attached to other axially adjacent rings via connectors 116 as described in various embodiments herein. The stabilizing stent has a covering on at least a portion of its surface to prevent blood flow from crossing from the left atrium to the left ventricle and bypassing the valve leaflets (as described in various embodiments herein). It is understood that the stabilizing stent can have a structure that is similar to stents currently used in the coronary or peripheral vasculature, used in the aorta, or used as frame structures for current TAVR devices, either SE or BE. Alternatively, the stabilizing stent can incorporate the combination BE/SE features as described above, and can incorporate expansion limiters as described above.

In the embodiments of FIGS. 32A-32D the upper bulb 82 and lower bulb 86 are formed from a SE material such as NiTi and have a diameter that is larger than the mitral annulus 20 (i.e., 3.5 cm, range 2.5-4.5 cm). The waist 84 of a SE or BE is designed to expand outwards to make contact with the mitral leaflets (native valve leaflets 22) and provide outward force to eventually expand outwards (minutes to hours) to meet the mitral annulus 20. The stabilizing stent (stent 66 comprising stabilizing portion 88) of FIGS. 32A (unexpanded configuration) and 32B (expanded configuration) is formed having strut lengths S in the waist 84 that are shorter than the strut lengths in the upper bulb 82 and lower bulb 86; the shorter strut length in the waist 84 are not able to expand outwards as far as the longer bulb struts resulting in an expanded stent as shown in FIG. 32B that has a waist 84 with a smaller diameter than the upper bulb and lower bulb. The waist 84 centers the stabilizing stent across the mitral annulus 20; the upper bulb 82 prevents the stabilizing stent from migrating or embolizing toward the left ventricle; the lower bulb 86 prevents embolization or migration of the stabilizing stent toward the left atrium. The stabilizing stent is formed, for example, of NiTi or stainless steel using laser machining of a metal tube. The NiTi stent would then be thermally treated using standard NiTi thermal processing to form the final equilibrium shape found in FIG. 32B. The stent is then collapsed or compressed to form a smaller diameter configuration as shown in FIG. 32A for delivery to the patient via a sheath 144. In one embodiment of a SE stent the equilibrium (upon free expansion) waist diameter 96 (which is 2-8 mm smaller than the bulb diameter) can be entirely processed into the stent shape or structure via thermal processing of the NiTi even though the strut lengths are the same or only slightly different between the waist and the bulbs. Some elements just described are not shown in FIGS. 32A-32D but are shown in other figures herein for clarity of illustration.

The embodiment of FIGS. 32C and 32D show a stabilizing stent having a waist 84 with similar length of waist struts to the length of the bulb struts but the bulb strut angle 316 with respect to the axis is less than the waist strut angle 314. Upon expansion of the stent from its unexpanded configuration shown in FIG. 32C to its expanded configuration shown in FIG. 32D the waist strut angle 314 becomes almost 90 degrees with respect to the axis and the waist struts are unable to expand further, thereby providing the waist with a smaller diameter than the bulb diameter.

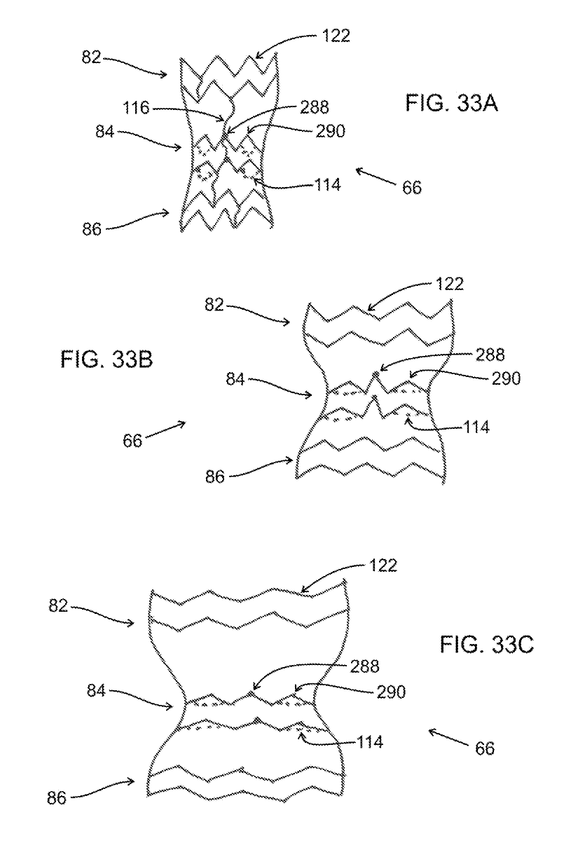

The embodiment shown in FIGS. 33A-33C show a stabilizing stent having a SE upper bulb 82 and SE lower bulb 86 and a combination BE/SE waist 84 having BE hinges 288 in series with SE hinges 290 as described in earlier embodiments. Expansion limiters 114 are located across SE hinges 290 to prevent the SE hinges 290 from expansion beyond the limit provided by the expansion limiter 114 as described in earlier embodiments. In its unexpanded configuration shown in FIG. 33A the expansion limiter 114 has a curved or slack configuration that would allow further expansion of the SE hinge 290. Located adjacent to the SE hinge 290 and in series with the SE hinge 290 are BE hinges 288. The BE hinges 288 have strength to resist deformation bending within the sheath 144 due to forces generated by the compression of the SE hinges 290. Stent 66 is delivered within a delivery sheath (sheath 144) that holds the stabilizing stent in its unexpanded configuration for delivery to the site of mitral valve implant. Stent 66 is released from the sheath 144 using a pusher rod in a manner consistent with delivery of most SE stents or stent-graft systems; alternate delivery systems can be used to deploy the stent-valve. Connectors 116 can be used to connect zig zag rings or ringlets of the stent 66. Connectors 116 can be curved or nonlinear metallic elongated elements that are easily bent during expansion deformation and can be contiguous with the wall structure of the ringlets 318 connecting one ringlet 318 to a neighboring ringlet 318. Upon release from the sheath 144, the stabilizing portion 99 of stent 66 assumes an intermediate configuration as shown in FIG. 33B. The SE hinges 290 have expanded outwards to their fullest extent and are limited for further expansion by the expansion limiters 114. The diameter of the waist 84 is significantly smaller (i.e., by at least 20%) than the diameter of upper bulb 82 and lower bulb 86. Stent 66 can be left in place at this point with the waist 84 located adjacent mitral annulus 20 (i.e., with the same axial position with the mitral annulus), the upper bulb 82 located in the left atrium 24 just above mitral annulus 20, and the lower bulb 86 located adjacent the native valve leaflets 22, pushing them outwards. Alternatively, the stabilizing portion 88 can be further dilated or post dilated using a balloon or other mechanical dilation means. The waist 84 is able to expand further to a larger diameter shown in FIG. 33C as the BE hinges 288 expand outwards under the dilating force of the dilation balloon. It is understood that the upper bulb 82 and lower bulb 84 portions of the stabilizing portion 88 can be formed with the same combination BE/SE stent wall structure as found in waist 84 if desired.

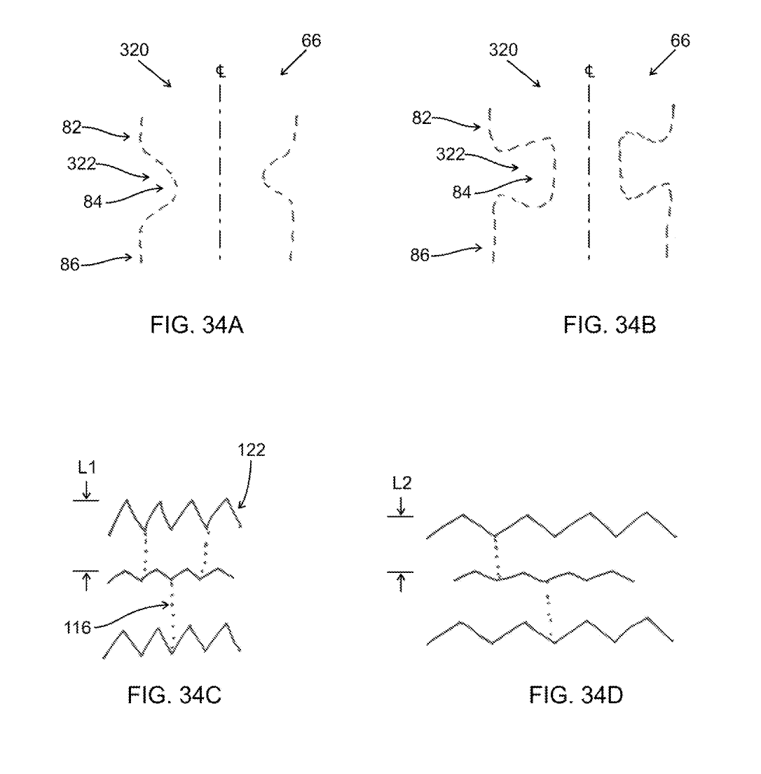

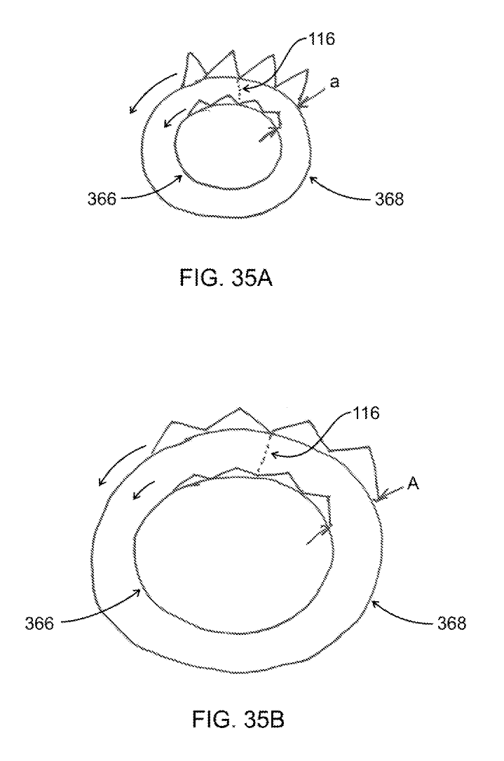

One embodiment of a stent 66 of the present invention is pinch stent 320 shown in FIGS. 34A-35B; pinch stent 320 can be formed from SE or BE materials. For a SE stent formed from NiTi or other elastic metal the shape of the smaller diameter waist can be thermally processed into the stent such that it tends to elastically return to the fully expanded diameter having the pinched shape. As shown in FIG. 34A, waist 84 has a significantly smaller diameter (i.e., at least 20% smaller diameter) than the upper bulb 82 and lower bulb 86 when it is not fully deployed. As the pinch stent 320 is deployed further to a larger diameter, upper bulb 82 moves toward lower bulb 86 in an axial direction to form a pinch region 322 where the axial distance between the upper bulb 82 and lower bulb 86 or the waist length 334 at the outer perimeter of pinch stent 320 is reduced in axial length; this reduced length can be less than the annulus length 336. Thus the pinch stent 320 applies a force onto mitral annulus 20 to compress the mitral annulus in an axial direction. This axial compression does not apply significant outward force onto mitral annulus 20 that could generate further mitral dilation. The pinch region 322 is intended to be placed across the mitral valve annulus such that upper bulb 82 is located in the left atrium and lower bulb 86 is located in the left ventricle adjacent the native leaflets. To accomplish the axial movement of the upper bulb 92 and lower bulb 86, individual zig zag ringlets 318 of the bulbs are connected to axially adjacent ringlets 318 of the waist via connectors 116 as shown in FIGS. 34C and 34D. The connectors are metallic elements that are flexible and contiguous with the stent wall structure; connectors 116 join one ringlet 318 with a neighboring ringlet 318. The zig zag ringlets 318 of the waist 84, for example, have smaller length struts than the those of upper bulb 82 and lower bulb 86. As the stabilizing stent is expanded in diameter, the connectors 116 force the ringlets 318 of the upper bulb 82 and lower bulb 86 to move nearer to the ringlets 318 of waist 84 as shown in FIG. 34D. The axial distance between ringlets of the bulb and waist is reduced from distance L1 to distance L2. Further examination of the stabilizing stent from an end view is shown in FIGS. 35A and 35B. Only a few zig zags of waist ringlet 366 and bulb ringlet 368 are shown, with circles indicating the pattern continues around. The stent in its non-fully expanded configuration is shown in FIG. 35A having a radial distance between a waist ringlet 366 and a bulb ringlet 368 of distance, a. As the pinch stent 320 is expanded in diameter, the connectors 116 increase in radial length (distance in the radial direction) to a distance of, A; this increase in radial distance corresponds to a reduction in axial distance from L1 to L2 which results in a pinching of waist 84 and reduced distance between upper bulb 82 and lower bulb 86 (waist length 334).

Release and deployment of pinch stent 320 is as follows. Pinch stent 320 is introduced using a delivery catheter. Partial release of pinch stent 320 allows waist portion 84 to expand outward to meet mitral annulus 20. Further release of upper bulb 82 and lower bulb 86 of pinch stent 320 allows upper bulb 82 and lower bulb 86 to expand somewhat, to a diameter larger than that of waist 84 to allow the stent to form a pinching shape with the narrower waist 84 aligned with mitral annulus 20. At this stage, pinch stent 320 can be repositioned in an axial direction more distally or proximally if necessary. Upon complete release of upper bulb 82 and lower bulb 86, the stent expands further, causing the pinching action as described above. Thus, pinch stent 320 is anchored at mitral annulus 20, and can provide a stable structure for placement of replacement valve 52.

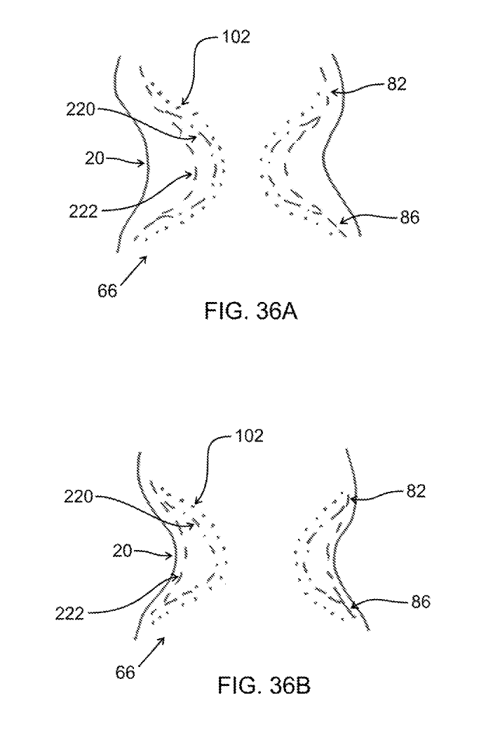

FIG. 36A-36B shows another embodiment for the stabilizing stent of the present invention. Stent 66 comprises inner stent structure 222 and an outer stent structure 220 along with upper bulb 82 and lower bulb 84. Stent 66 can be used as a component of adapter 64 that allows placement of a smaller diameter stent-graft or TAVR device within the inner stent structure 222; adapter 64 can contain temporary leaflets 190 made of a polymer and having a thin wall thickness and low profile; alternatively, adapter 64 can comprise a cylinder-like wall structure having axial fibers 180 as described above such as in FIG. 21A. Alternatively, the stent 66 can be firmly attached to a housing or tract element 90 that contains permanent replacement leaflets 100 similar to those found in current BE or SE TAVR devices having trileaflet valves formed from tissue sources.

The embodiment of FIGS. 36A and 36B show a stabilizing stent having an inner stent structure 222 that is SE and an outer stent structure 220 that is formed from a combination BE/SE structure as described above. Placement of stent 66 across mitral annulus 20 can be accomplished using a sheath 144 that contains stent 66 in a smaller diameter configuration. Upon release of stent 66 from sheath 144 stent 66 expands outwards such that the SE upper bulb 82 and lower bulb 86 expand into contact with the left atrium and with the mitral leaflets mitral in the left ventricle; waist 84 is positioned adjacent to the mitral annulus 20 as shown in FIG. 36A. Waist 84 of the outer stent structure 220 in this embodiment is not required to come into direct contact with mitral annulus 20 along its entire perimeter upon initial release of the stent 66 from sheath 144. If additional dilation of stabilizing portion 88 of stent 66 is needed to ensure direct contact of the outer stent structure 220 with mitral annulus 20 (as shown in FIG. 36B), stent 66 can be post dilated with a balloon catheter or other mechanical expansion mechanism to cause the outer stent structure 220 to expand further via a BE plastic deformation of BE hinges 288 of outer stent structure 220. Inner stent structure 222 will expand outwards elastically during balloon expansion but will return or compress in diameter to the nominal diameter of SE inner stent structure 222 determined by its equilibrium diameter. This embodiment provides the advantage that outer stent structure 220 does not provide an continued outward force against the mitral annulus; this advantage is particularly important for treating those patients who suffer from functional mitral regurgitation which results from expansion of the ventricular myocardium and causes dilation of the mitral annulus. Further expansion force against the mitral annulus can result in further dilation of the mitral annulus leading to potential for perivalvular leaks, centro-valvular leaks, and poor coaptation of the implanted replacement valve leaflets leading to mitral regurgitation, premature leaflet failure, or possible stent-valve migration.

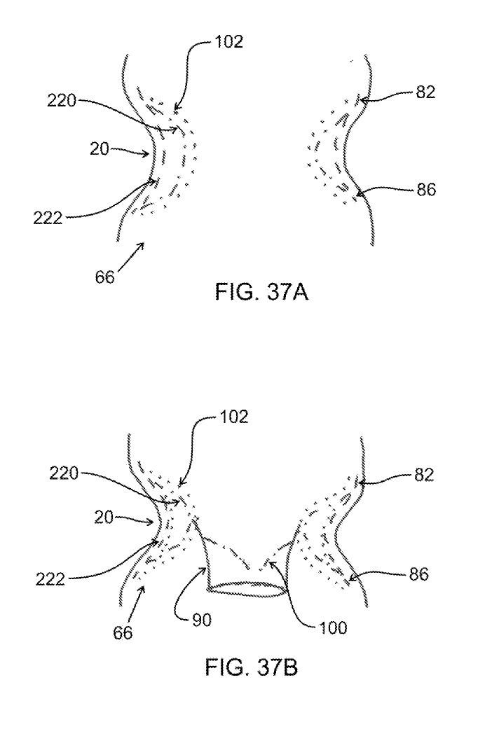

FIGS. 37A and 37B show another embodiment of a stent 66 that comprises inner stent structure 222 and outer stent structure 220. In this embodiment outer stent structure 220 is a SE stent and the inner stent structure is a combination BE/SE stent with expansion limiters 114 or a SE stent with expansion limiters 114. Due to the SE components of both the inner stent structure 222 and outer stent structure 220, the present embodiment can be delivered via release from sheath 144, similar to the approach presently used for many SE stent and stent-graft systems. Contiguous with outer stent structure 220 are SE upper bulb 82 and SE lower bulb 86 of stabilizing portion 88. Covering 102 is also attached to inner stent structure 222, outer stent structure 220, or both; covering 102 also can be applied to upper bulb 82 and lower bulb 86 to ensure that perivalvular leakage is minimized. Upon release of stent 66 from sheath 144, the outer stent structure 220 expands outwards to contact the mitral annulus and form a tight seal to prevent blood leakage. Inner stent structure 222 is allowed to expand to an equilibrium diameter that is significantly smaller than the mitral annulus diameter (at least 20% less in diameter). The upper bulb 82 and lower bulb 86 expand outwards to a diameter that is larger than the mitral annulus diameter. The lower bulb 86 holds native valve leaflets 22 outwards and prevents migration of stent 66 retrogradely toward the left atrium; upper bulb 82 expands against the wall of the left atrium to make a seal against blood leakage and to ensure the device does not migrate antegrade. Attached to stent 66 is a housing or tract element 90 as shown in FIG. 37B. The housing can be formed from fabric 182 as described above. Alternatively, the housing can be formed from a stent wall structure having a cylindrical or conical shape as described above. The stent wall structure can be a SE stent wall structure or it can be a SE wall structure having expansion limiters 114 as described above. Temporary leaflets 190 can be located within the housing for embodiments of adapter 64. Alternatively, permanent replacement leaflets 100 can be located and attached within tract element 90 to allow this structure to function as a one-step TMVR device.

The embodiments shown in FIGS. 36A-37 having an inner stent structure 222 and outer stent structure 220 can be formed from several methods. For example, additive manufacturing can be applied to deposit NiTi or other metal or material in a 3D manner to form the hinges 282 and stent struts 118 of stent 66; the junctions of the inner stent structure 222 and outer stent structure 220 can, for example, be made contiguously in a manner that is not subject to potential fracture or fatigue over time. Alternatively, known processing methods can be used to join the inner stent structure 222 and outer stent structure 220 together such as by welding, brazing, bonding, suture attachment or other methods used in the medical device industry.

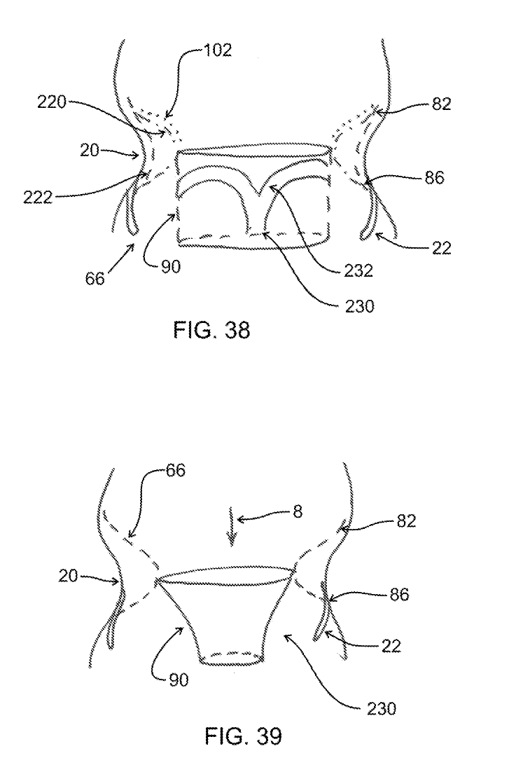

One embodiment for the present TMVR system having a housing or tract element 90 attached to stabilizing portion 88 of stent 66 as described is shown in FIG. 38. One concern for a TMVR system is the formation of thrombus in regions of blood stagnation; such regions can occur between native valve leaflets 22 and the tract element 90 or between the native valve leaflets 22 and left ventricular lateral wall 32. Such thrombus formation can embolize over time and cause a stroke or CVA in the patient. One of the key aspects of all embodiments of the present invention described herein is that the diameter of the housing or tract element 90 that holds the replacement leaflets 100 is significantly smaller than the diameter of the mitral valve annulus (i.e., at least 20% smaller than the diameter of the mitral annulus). The smaller diameter provides space between tract element 90 and left ventricular lateral wall 32; this space allows blood to continually flow past and cleanse the surfaces of the myocardium and the outer wall of the native leaflets to reduce thrombus formation in this area. To ensure that the inner surface of the native leaflets are similarly cleansed without stagnation regions the housing of one embodiment of the present invention contains open space without a covering over the tract element except where the replacement leaflets are attached to the tract element along a crown-shaped attachment line and continuous to where the tract element attaches to the stabilizing portion. Blood is thereby able to move through the open spaces in these regions and structures to ensure that the inner surface of the native leaflets are cleansed by blood flow without significant stagnation area. Thus significant thrombus formation is reduced or avoided at these structures and regions.

FIG. 39 shows another embodiment of the tract element that holds the replacement leaflets. In this embodiment the housing or tract element (see prior embodiments) is formed with a conical shape where the distal opening located downstream and within the left ventricle has a smaller diameter than the housing or tract portion of the stent located upstream. This conical shape provides a hemodynamic shape that favors the direct contact of the native leaflets against the outside of the housing or tract element during systole. The outside surface of the native valve leaflets are provided maximal space for cleansing the tissues and nearby structures while minimizing stagnation regions. The inner surface of the native valve leaflets are held against the outside surface of the tract element so that healing can occur over time to hold the native leaflets firmly against the outside surface of the housing. The conical shape for the tract element also provides a stable hemodynamic flow pattern for antegrade blood flow through the housing and replacement leaflets in the TMVR device (replacement valve 52).

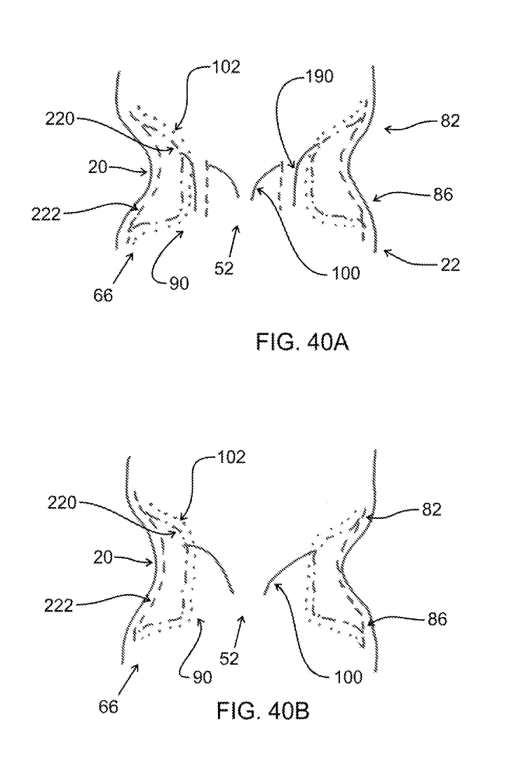

FIGS. 40A and 40B show an embodiment for stent 66 that has an inner stent structure 222 with a cylindrical portion that is long enough in axial length to accommodate temporary leaflets 190 (approximately 0.75 inches, range 0.5-1.5 inches). The embodiment of FIG. 40A is an adapter 64 that accommodates the secondary implantation of replacement valve 52 such as a stent-valve, TAVR device tract stent, or modified TAVR device (i.e., modified TAVR stent frame or housing diameter, TAVR frame length, presence or lack of a full skirt) within inner stent structure 220 of stent 66. The TAVR device or secondarily-implanted stent-valve device can comprise SE or BE material for the tract element 90. The inner stent structure 220 of the present embodiment is a combination BE/SE stent or a SE stent with expansion limiters 114 across the SE hinges 290 as described above; the inner stent structure 220 has a cylindrical region to serve as a landing zone for a stent-valve or valve leaflets; the outer stent structure 222 is a SE stent. Temporary leaflets 190 are located within the inner stent structure 220 and are attached to inner stent structure 220 along a crown-shaped attachment line 232 similar to that described above for other embodiments. Covering 102 is located on the surface of outer stent structure 222 and continues onto upper bulb 82 and lower bulb 86 of stabilizing portion 88 of stent 66. After a first step of delivering stent 66 and tract element 90 with temporary leaflets 190, a smaller diameter TAVR device or smaller diameter stent-valve device is delivered as a second step. For a balloon-expandable TAVR device or small diameter stent-valve, it can be expanded into direct contact with inner stent structure 222 of the stabilizing stent. The TAVR device or small diameter stent-valve will make contact with the inner combination BE/SE stent and cause it to expand as necessary to fit the diameter of the TAVR device or small diameter stent-valve to make a tight seal that will prevent leakage and prevent migration. The use of adapter 64 will reduce the profile of the overall system because placement of replacement valve 52 occurs in two steps, with each component of the implant having a smaller profile than a combined device having both the stabilizing stent and the stent-valve. The profile and wall thickness of the temporary leaflets 190 can be significantly smaller than that of a typical tissue valve leaflet, such as half a thick (i.e., 0.0003-0.001 inch).

Tract element 90 can comprise a fabric that is a woven, knitted, or film material as described earlier. The fabric, which is serving as a housing that provides the landing zone for implanting a stent-valve can be of a fixed diameter formed from a nondistendable material and serve to hold the stent-valve at a specific diameter of expansion that is significantly smaller (i.e., at least 20% smaller) than the diameter of the mitral valve annulus. Alternatively, the covering or housing can be an expandable covering or housing and can expand in diameter as the stent-valve is expanded within it. In this embodiment, the implanted stent-graft is pushed outwards to apply a radial force through the housing and against the inner stent structure to form a frictional force that holds the stent-graft from migration.

FIG. 40B is similar to FIG. 40A except that replacement leaflets 100 are attached to the inner stent structure 222. The attachment of the leaflets is similar to that described earlier; the attachment follows a crown-shaped attachment line 232 with the leaflet commissures located at the outlet end of the cylindrical portion of the inner stent structure 222. The inner stent structure 222 is formed from a combination BE/SE stent configuration with expansion limiters 114 or with a SE stent that has expansion limiters 114 across the SE hinges 290. The outer stent structure 220 is a SE stent. Covering 102 is located on the surface of the inner stent structure 222 and upper bulb 82 and lower bulb 86; covering 102 can also be placed on the surface of the inner stent structure 222. In this embodiment the entire device including the stent 66 and replacement leaflets 100 are delivered in one step by release from sheath 144.

Embodiments of the present invention include devices that have a stent that forms a seal with the mitral annulus and the tissues upstream and downstream of the mitral annulus. In some embodiments, the invention is an adapter with a stabilizing portion and a tract element, placed in a first step, that allows a second step of placing a TAVR device, a modified TAVR device, or a small diameter stent-valve within the adapter. In other embodiments, replacement leaflets are already attached to tract element 90 which is in turn already attached to stent 66 and the implantation is performed as one step. Each embodiment presented can be used with or combined with features of other embodiments of the present invention to provide an implanted intravascular mitral valve of the present invention.

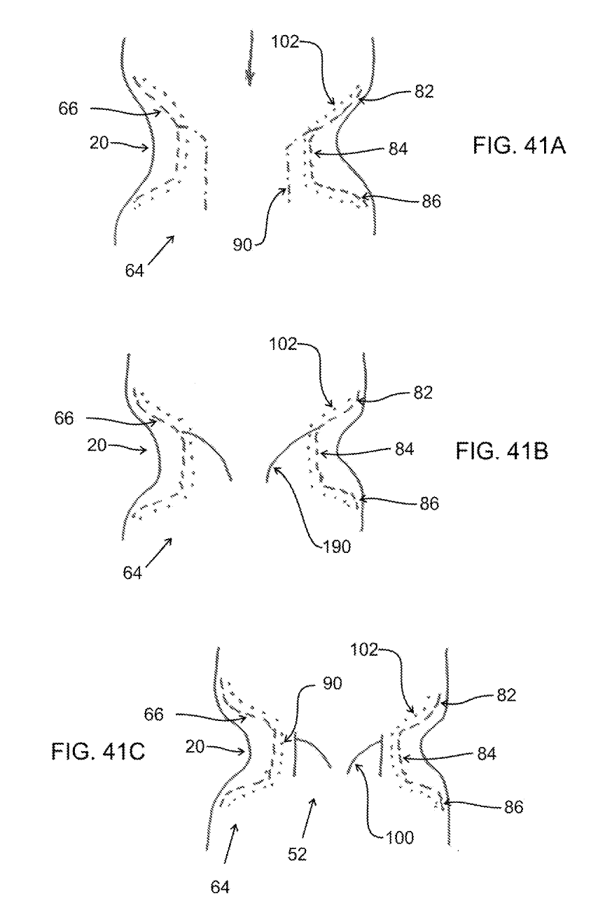

FIGS. 41A-41C show an additional embodiment for adapter 64 of the present invention; adapter 64 is intended to provide a low-profile initial implant across a dysfunctional native mitral valve and provide the necessary structure to allow a second implant within the adapter of a TAVR device or an implanted stent-valve having a significantly smaller diameter (i.e., at least 20% less) than the diameter of the mitral valve annulus. In this embodiment stent 66 comprises stabilizing portion 88 which comprises waist 84, upper bulb 82, and lower bulb 86, and is positioned across the mitral annulus such that waist 84 is adjacent the mitral annulus 20. The diameter of waist 84 of stabilizing portion 88 is significantly smaller (i.e., at least 20% less) than the diameter of the mitral valve annulus, and significantly smaller than the diameter of the upper bulb 82 and lower bulb 86. Waist 84 has a cylindrical shape for at least a portion (at least 5 mm) of the waist; the waist extends from 5-30 mm in axial length.

Upper bulb 82 and lower bulb 86 of stabilizing portion 88 are formed from a SE or elastic material such as NiTi. Waist 84 is formed from a SE material that has expansion limiters 114 as described above; the SE material allows stabilizing portion 88 of stent 66 of adapter 64 to be delivered via release from sheath 144 as described above. Expansion limiters 114 allow the secondary implantation of a stent-valve within waist 84 of adapter 64 such that waist 84 of adapter 64 expand only a certain desired amount, retaining waist 84 at a diameter that is significantly smaller (i.e., at least 20% smaller) than the diameter of the mitral valve annulus. Waist 84 of the stabilizing portion 88 of adapter 64 can alternatively be formed from a combination structure having both SE elements and BE elements in series as described above, such that waist 84 of adapter 64 can expand further by undergoing plastic deformation of the BE elements to hold frictional force against the secondary stent-graft to prevent migration of the stent-graft and prevent perivalvular leaks; waist 84 of the combination SE/BE stent can have expansion limiters 114.

Waist 84 can have a cylindrical shape for an axial distance of 5-30 mm to provide a landing zone for the delivery of the secondary TAVR device or secondary stent-valve. Upper bulb 82 has a conical or tapered shape which extends from the smaller diameter waist 84 (i.e., at least 20% smaller than the upper bulb end) to a larger diameter end of upper bulb 82. Lower bulb 86 is tapered to extend from the smaller diameter waist 84 to a larger diameter that holds the native valve leaflets 22 outward and helps prevent migration of adapter 64 into the left atrium.

Covering 102 is attached to the surface of upper bulb 82 and lower bulb 86 to prevent blood leakage past the closed valve from the LV to the LA; covering 102 can also extend throughout the axial length of waist 84. Covering 102 as shown in the embodiment of FIGS. 41A and 41B is attached to tract element 90 which provides the site for implantation of a TAVR device or stent-valve in a second step.

Tract element 90 of the embodiment of FIG. 41A extends downstream through waist 84 at a diameter that is at least 20% smaller than the mitral valve annulus; the tract element 90 can extend downstream beyond the downstream end of waist 84. Tract element 90 can be attached to waist 84; alternatively tract element 90 can be a separate cylindrical tubular structure that is attached at its inlet end to stent 66 or to covering 102. Tract element 90 extends in an axial direction for a length ranging from 5-40 mm. The tract element 90 of this embodiment can be comprised of a fabric 182 and can have axial fibers 180 extending along its perimeter as shown in FIGS. 7A-7D and 21A-21B. In this embodiment tract element 90 can serve as a temporary valve prior to subsequent implantation of a stent-valve. The tract element in one embodiment is an expandable structure formed from an elastomeric material such as NiTi, for example, and with an expandable covering, for example, as described above. Upon implantation of the stent-valve, such as a BE TAVR device, the tract element 90 expands in diameter until the tract element makes contact with waist 84 and the stent-valve is restricted from further expansion by the diameter of waist 84.

In an alternate embodiment also shown in FIG. 41A, the tract element 90 can be formed from a nondistendable material such as PET and can retain a diameter that is significantly smaller (i.e., at least 20% smaller than) the diameter of the mitral valve annulus. Upon implantation of the stent-valve within the waist 84 of adapter 64, waist 84 holds the stent-valve from expansion beyond a diameter that is significantly smaller than the mitral valve annulus.

A further embodiment for covering 102 and tract element 90 is shown in FIG. 41B. In this embodiment covering 102 is attached to upper bulb 82 and lower bulb 84 of stabilizing portion 88 of stent 66. Tract element 90 has a cylindrical landing zone formed from fabric 182 that prevents blood leakage through its walls and thereby prevents perivalvular leaks. Fabric 182 of tract element 90 is attached to covering 102 for upper bulb 82 and lower bulb 86; covering 102 and fabric 182 can be contiguous materials and can be materials such as those described above for covering 102 or for fabric 182. In this embodiment temporary leaflets 190 as described above are located within attached to the inner surface of tract element 90. In one embodiment tract element 90 is expandable such that upon subsequent implantation of the stent-valve, the tract element is able to expand outwards to a larger diameter and the underlying waist 84 restricts further expansion of the stent-valve thereby preventing migration of the stent-valve and also preventing perivalvular leaks.

As shown in FIG. 41C replacement valve 52, such as a stent-valve or TAVR device, has been implanted within the lumen of adapter 64 adjacent the inner surface of waist 84. The tract element 90 as described in this embodiment is expandable and hence is able to expand outwards in a radial direction and make contact with waist 84. In some embodiments, temporary leaflets within tract element 90 are also able to extend in diameter while still maintaining coaptation with neighboring leaflets. In one embodiment tract element 90 is attached to waist 84 of stabilizing portion 88 of stent 66. Waist 84 prevents excessive expansion of the stent-graft such that it is of a significantly smaller diameter (i.e., at least 20% less) than the diameter of the mitral annulus.

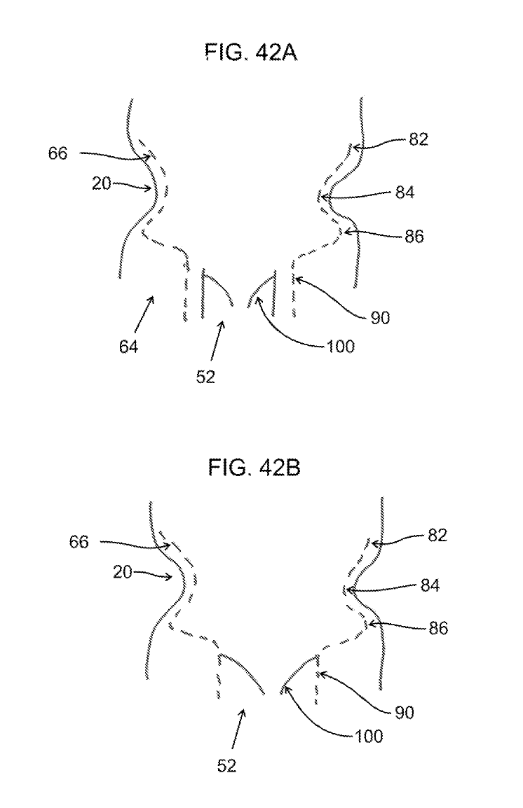

An additional embodiment for the present invention is shown in FIG. 42A; this embodiment provides an adapter 64 to be implanted at the mitral annulus to allow a replacement valve 52 which in this case is a stent-valve to be implanted within adapter 64 and provide a structure to hold the stent-valve in position across the mitral valve. The adapter 64 comprises a stent 66 with stabilizing portion 88 having upper bulb 82, lower bulb 86 and waist 84; upper bulb 82 is placed in the left atrium, waist 84 is placed adjacent the mitral annulus, and lower bulb 86 extends into the left ventricle and in contact with the native valve leaflets 22 of the mitral valve. Waist 84 of stabilizing portion 88 is 3-8 mm smaller in diameter than upper bulb 82 or lower bulb 86. Attached to lower bulb 86 is tract element 90; tract element 90 can be contiguous with stabilizing portion 88 or it can be bonded, welded, or otherwise attached to stabilizing portion 88. Tract element 90 has a covering 102 (not shown) attached to its surface; other portions of stent 66 can also have covering 102. Tract element 90 provides a housing for holding a stent-valve; temporary leaflets in tract element 90 provide short-term function prior to delivery of the stent-valve that is implanted within the tract element 90. Tract element 90 can have a cylindrical portion or cylindrical housing that is of a significantly smaller diameter than waist 84 (at least 20% lower diameter) to hold a stent-valve that is expanded outward and into contact with the cylindrical portion of tract element 90.

Stabilizing portion 88 has at least some SE character such that it can be released from sheath 144 and can expand into place and locate the waist 84 adjacent to the mitral annulus 20. Upper bulb 82 and lower bulb 86 can be formed from an elastic metal such as NiTi, elgiloy, or other material having self-expanding character. Waist 84 can be formed from a SE metal and can have expansion limiters 114 that extend across at least some of SE hinges 290 and attach to stent struts 118 of stent 66 as described above. The presence of expansion limiters 114 allows waist 84 to be expanded via a balloon as a post dilation step to ensure that waist 84 is fully deployed yet will not expand in an unrestricted manner to exert excessive outward forces against mitral annulus 20 on an ongoing basis. Waist 84 can also be formed with a combination of SE and BE structure with at least some SE hinges 290 and BE hinges 288 located in series as described above. This combination structure allows waist 84 to be post dilated via a balloon, for example, to further expand waist 84 into contact with mitral annulus 20.

Tract element 90 of stent 66 can be formed with a metal that exhibits at least some SE character such that the tract element 90 opens up to provide function of a temporary valve upon delivery from sheath 144 as described above. Temporary leaflets 190 provide function of a temporary valve; temporary leaflets 190 are formed from a thin polymeric film or other thin material that is strong enough to support the forces applied during systole. Tract element 90 can be formed from a SE metal such as NiTi or other elastomeric metal. Tract element 90 can comprise SE hinges 290 with expansion limiters 114 that extend across at least some of the SE hinges 290; expansion limiters 114 serve to limit the expanded diameter of the tract element 90 within which replacement valve 52 such as a stent-valve is being implanted. Thus the stent-valve can expand outwards into contact with tract element 90 and be held by a tract element 90 that is restricted from further expansion. Tract element 90 or at least a cylindrical portion of tract element 90 can alternatively comprise a combination of SE and BE hinges located in series as described above. Upon delivery of replacement valve 52 (such as a TAVR device or other stent-valve) to tract element 90, the stent-valve can be post dilated, expanding tract element 90 further, but further expansion of tract element 90 is limited by expansion limiters 114 located across SE hinges 290 of the stent structure.

It is noted herein that the expansion limiters 114 can themselves comprise a BE material such that expansion of stent 66 comprising expansion limiters 114 via a post dilation can allow further expansion of stent 66 to a controlled extent; inherent expansion forces provided by self-expanding aspects of the stent itself can be terminated by the use of such expansion limiters.

FIG. 42B is a TMVR device having a stent 66 that is similar to in structure to the embodiment of FIG. 42A. Here tract element 90 comprises a cylindrical portion that is significantly smaller than waist 84 as described above in the embodiment of FIG. 42A. Tract element 90 of this embodiment contains permanent mitral valve replacement leaflets 100 attached to the tract element 90. The device is implanted across the mitral annulus 20 via release from sheath 144 and begins operating upon delivery. The TMVR device can be post dilated to provide better approximation of stabilizing portion 88 with mitral annulus 20 and to ensure that native valve leaflets 22 are pushed to the side to allow proper functioning of replacement leaflets 100.

As shown in FIG. 42B, as for any of the SE embodiments of the present invention, both the one-step TMVR devices and the adapter 64 for placement of a stent-valve in a second step, are able to be repositioned after initial deployment in a position across the mitral valve if it is determined that the device is not properly positioned with waist 84 adjacent to mitral annulus 20. The upstream end of the stent 66 can be attached to an attachment means such as an attachment cord or wire or other attachment device for aid in retracting the device into sheath 144 for such repositioning. If the axial positioning of the stabilizing stent is not located properly, the stent can thereby be pulled back into sheath 144 from which it was deployed and the device can be repositioned across the mitral valve; alternately, the stent of the present invention can be pulled entirely back within the delivery sheath and can be removed from the patient if desired.

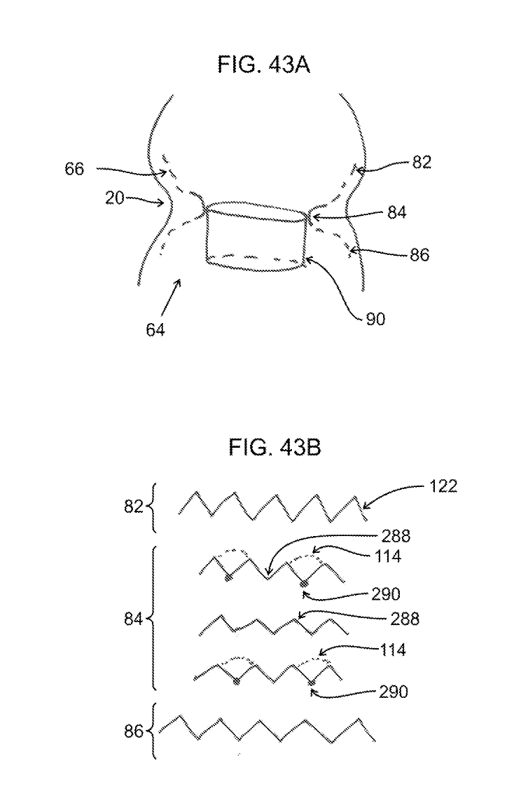

Another embodiment of the adapter of the present invention is shown in FIGS. 43A and 43B. Adapter 64 comprises a stabilizing stent 66 to which a housing (tract element 90) is attached. The housing can be a fabric 182 with axial fibers 180 or it can be a stent-like frame; the housing can have temporary leaflets 190 attached to the housing or the housing itself can perform the function of a temporary valve as described above. As shown in FIG. 43A, for example, the housing is a cylindrical housing that is attached to waist 84 of stabilizing portion 88 of stent 66; other configurations for the housing and stabilizing stent and their attachment to each other are anticipated. The stent structure for the stabilizing stent of this embodiment provides both a significantly smaller diameter (i.e., at least 20% smaller than the mitral annulus diameter) to which the housing is attached as well as providing direct contact of a portion of the waist with the mitral annulus; this direct contact will ensure that the axis of adapter 64 is well aligned with the axis of the mitral annulus and also ensures that the adapter is not able to migrate after it has been deployed.

The structure for upper bulb 82 and lower bulb 86 are comprised of a SE stent material such that they expand on the upstream and downstream sides of the mitral annulus upon deployment and provide for early functioning of the temporary valve located within or in conjunction with the housing of the adapter. Waist 84 of the stabilizing stent is comprised of two different portions of stent structure. A portion of the waist is formed from a SE material that forms a diameter for the waist that is significantly smaller than the diameter of the mitral annulus. This SE portion can be formed from one or more SE rings of zig zag NiTi material, for example. Another portion of the waist is formed from a combination BE/SE material that has BE hinges in series with SE hinges 290. The SE hinges 290 can have expansion limiters 114 located across the SE hinge 290 to restrict the SE hinges 290 from expansion beyond a specified limit. The combination BE/SE stent ringlets 318 can be configured such that they make direct contact with the mitral annulus at the upstream end of the mitral annulus and at the downstream end of the mitral annulus; the SE stent ring can be located at the center of the mitral annulus. The BE hinges 288 remain in a contracted condition that can support the forces imposed upon them during containment of the stent within sheath 144. Note that the structures shown in FIGS. 43B, 44B, and 45B cam also comprise connectors, to connect the separate ringlets 122 which may not be in this small illustrative section.

Upon retraction of sheath 144, the stabilizing portion 88 of stent 66 will expand outwards into position across the mitral annulus 20. Further expansion of waist 84 via a balloon dilation, will allow the BE portion of the waist 84 to expand further and place a portion of waist 84 into contact with mitral annulus 20. The SE portion of waist 84 will rebound back to a smaller diameter that is significantly (at least 20% smaller) smaller than mitral annulus 20.

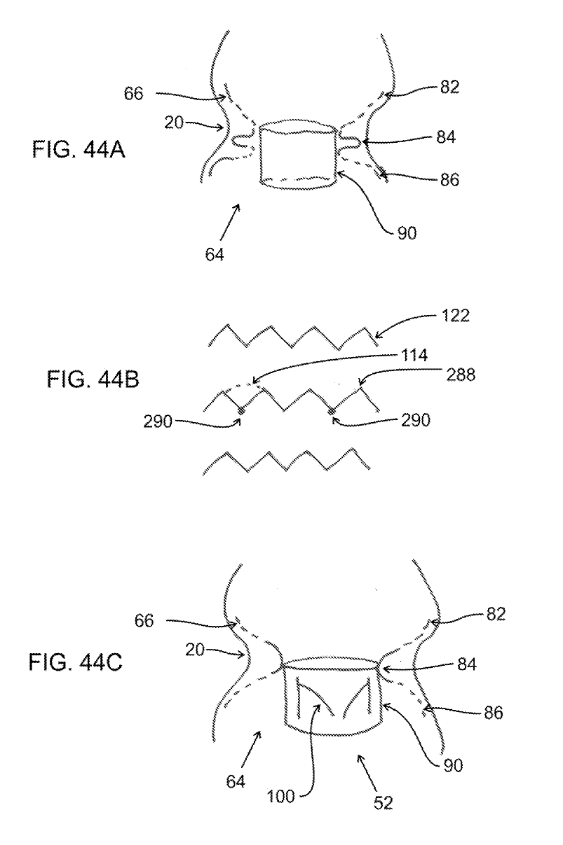

Alternate configurations for waist 84 can be employed as shown in FIGS. 44A and 44B. For example, one can place a combination BE/SE stent ringlet 318 with expansion limiters 114, for example, in the center of waist 84 such that the center of the waist 84 makes direct contact with the mitral annulus 20. SE stent rings can provide waist 84 with a smaller diameter that is significantly smaller than mitral annulus 20.

Following placement of adapter 64, a TAVR device or a smaller diameter stent-valve can be placed within the tract element 90 of adapter 64 as shown in FIG. 44C; the small diameter stent-valve can be a SE or BE device. Alternatively, instead of the present device being an adapter 64, the device can contain permanent replacement leaflets 100 attached to tract element 90 as described above.

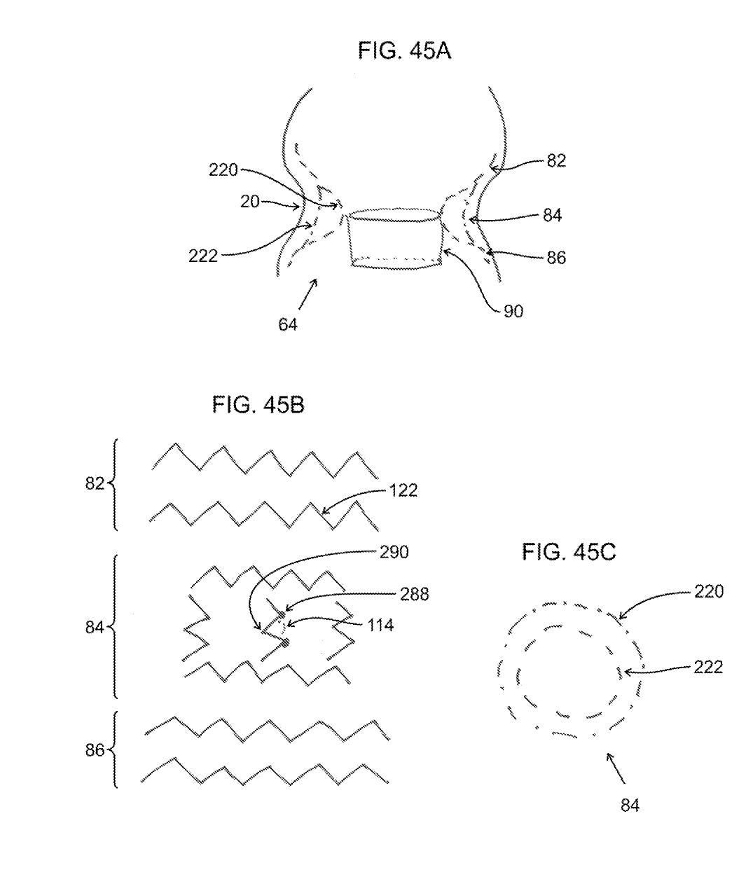

Another embodiment for an adapter 64 that is similar to that described in FIGS. 43A-44C for providing both direct contact of waist 84 of the stabilizing portion 88 of stent 66 with mitral annulus 20 while also providing a significantly (i.e., at least 20% smaller) smaller diameter of stent than the diameter of mitral annulus 20 is shown in FIGS. 45A and 45B. In this embodiment stabilizing portion 88 comprises inner stent structure 222 and outer stent structure. Inner stent structure 222 has SE stent elements; outer stent structure 220 has combination BE/SE elements. In this embodiment waist 84 has axially oriented zig zag elements; some elements are combination BE/SE elements that can have expansion limiters and others are SE elements. Upon delivery to the site of mitral annulus 20 and release from sheath 144 the stabilizing portion 88 of stent 66 expands outwards to locate itself across mitral annulus 20. Further expansion of the device via a balloon expansion will place the combination BE/SE elements into direct contact with mitral annulus 20. The SE elements will rebound and provide a smaller diameter frame to which the significantly smaller diameter housing (i.e. at least 20% smaller than the mitral annulus) can be attached.

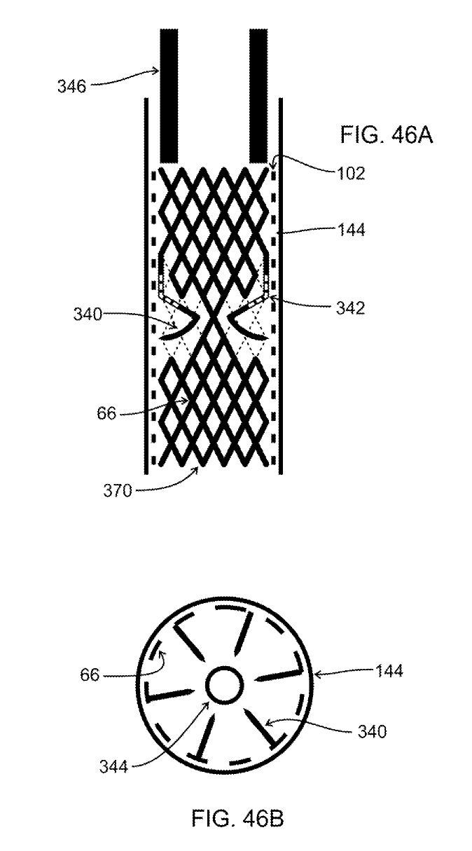

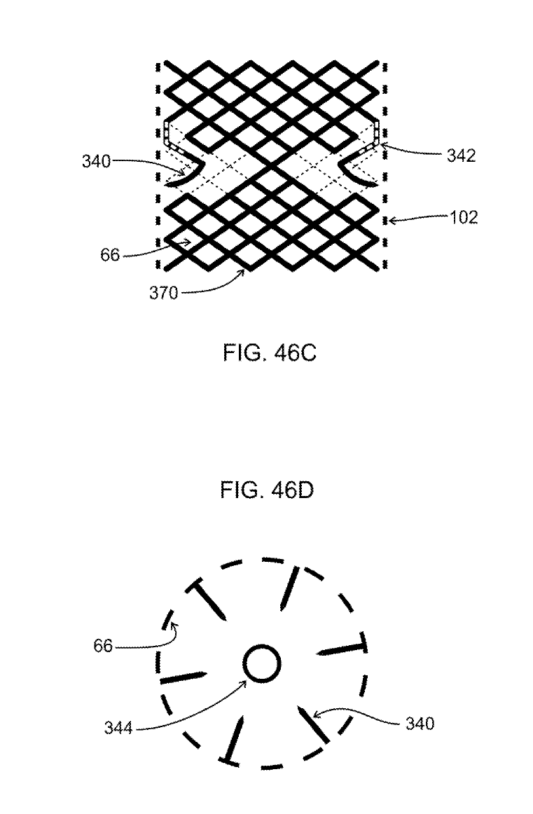

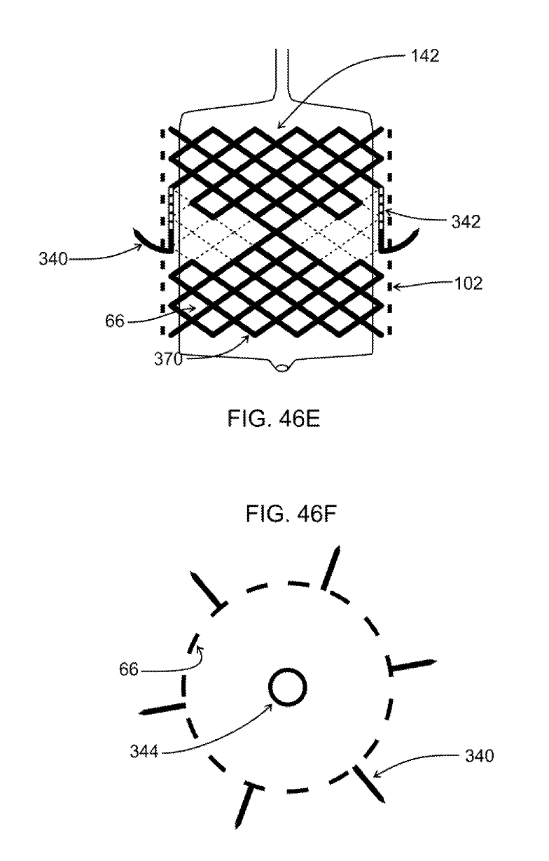

FIGS. 46A-46E show one embodiment for stent 66 with stabilizing portion 88 comprising barbs 340 located at waist 84 of the stent. The stent of this embodiment has a SE structure or a combination BE and SE hinge structure as described above. In FIGS. 46A and 46B sheath 144 holds stent 66 in a non-expanded configuration. Approximately 3-6 barbs (range 2-12 barbs) are retracted such that the barb does not extend beyond the generally cylindrical stent body 370; barbs 340 occupies much of the space located within the stent lumen as illustrated in FIG. 46B. Barb 340 is attached to stent 66 via barb hinge 342, barb hinge 342 can be a BE hinge formed via thermal methods or via geometric methods as described above. A central guidewire tube 344 is located along the central axis of the stent. Upon expansion release of stent 66 upon release from the sheath utilizing pusher tube 346 or other holding tube, stent 66 expands outwards as shown in FIGS. 46C and 46D. Stent 66 comes into contact with surrounding tissues such as mitral annulus 20 and native valve leaflets 22. Barbs 340 remain in a configuration that does not extend radially past the generally cylindrical body of stent 66 to avoid premature attachment to tissue and also to allow stent 66 to be recaptured by drawing stent 66 back into sheath 144 if needed. Covering 102 can be located as shown, for example, around the stent 66 (FIG. 46C), while allowing barbs 340 to extend through covering 102 when barbs 340 are deployed.

A dilation balloon such as balloon 142 can then be introduced within the lumen of stent 66 and expanded outwards to cause barbs 340 to extend outwards into the tissue of mitral annulus 20 or native valve leaflets 22 located adjacent the perimeter of stent 66 as shown in FIGS. 46E and 46F. For embodiments in which stent 66 is a combination stent with BE hinges 288 and SE hinges 290, the balloon 142 can also cause BE hinges 288 to deform to further expand stent 66 outwards and place stent 66 into further contact with the surrounding mitral annulus 20 and native valve leaflets 22. The expansion of BE hinges 288 confers to stent 66 a BE character that reduces the amount of outward force on mitral annulus applied over time that could contribute to unwanted annular dilation, especially annular dilation that occurs late over a period of weeks or months after implantation. Covering 102 could be located, for example, on the luminal side of the barb, if desired, such that the barb does not have to protrude through the covering, as shown in FIG. 46E.

The barbs 340 of this configuration (FIGS. 46A-46F) are directed primarily to hinge outwards upon extending outwards to prevent migration of the stent toward the LA during systole. It is understood that the barbs can be directed in the opposite direction to prevent migration towards the LV, or they can be directed in both directions to prevent movement of the stent in either direction as indicated in FIG. 46G. In another embodiment, stent 66 comprises fixation means comprising gripper elements 348 as shown in FIG. 46H which are attached and deployed in a manner similar to barbs 340 but which act to pinch tissue of mitral annulus 20 and/or native valve leaflets 22 rather than puncturing into the tissue. Fixation means comprising combinations of barbs 340, and gripper elements 348, in various combinations and orientations, are envisioned.

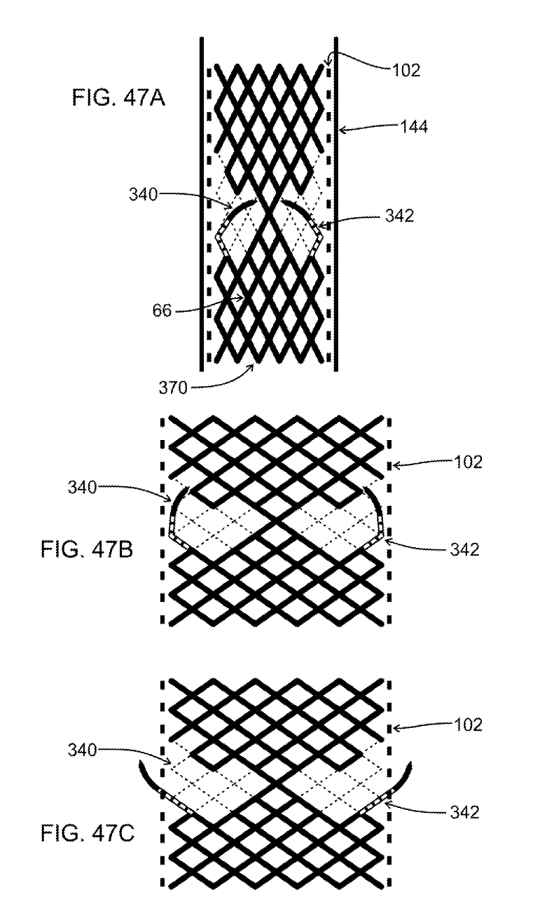

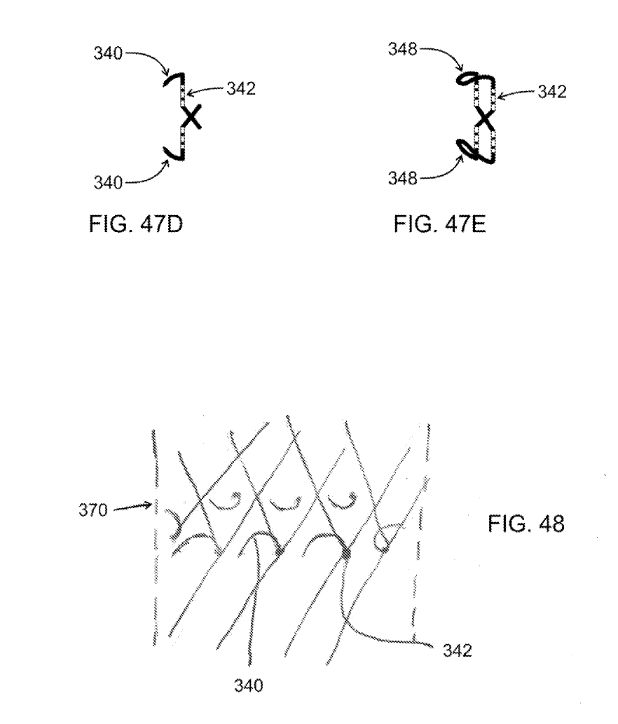

Another configuration for barb 340 of the present invention is shown in FIGS. 47A-47C. In this embodiment as shown in FIG. 46A, barbs 340 are again folded inward into the luminal space contained within the lumen of stent 66. Barbs can be attached, for example, to waist 84 of stabilizing portion 88 of stent 66. Alternatively, barbs 340 can be attached to lower bulb 86 and face upwards as shown in FIG. 47A; a similar set of barbs can be attached to upper bulb 82 and face downwards. If barbs 340 face the annulus from both the upper bulb and from the lower bulb, they pinch and capture tissue of mitral annulus 20 to prevent migration of stent 66 either toward the LA or towards the LV. Barbs can be formed from a NiTi alloy, stainless steel, or other material that can provide a sharp puncture of the annular or valvular tissue. Opposed orientation barbs as shown in FIG. 47D can be used to stabilize stent 66 and prevent movement either towards the left atrium or the left ventricle. Alternatively, a gripper elements 348 or non-sharp barb can be deployed to grab the annulus from each side rather than puncturing into the tissue as shown in FIG. 47D.

Although not shown on FIGS. 46A-47C for ease and clarity of illustration, stent 66 comprises upper bulb 82, waist 84, and lower bulb 86 as described above.

An alternate configuration for barbs 340 is shown in FIG. 48A-48B. In this embodiment the barbs are oriented around the perimeter of the SE or combination BE/SE stabilizing stent such that their main orientation is along the circumference of the stent. Barbs 340 are attached to hinges or struts of the stabilizing stent. Barbs 340 are attached to the stabilizing stent via BE barb hinges 342. Upon release of the stabilizing stent from sheath 144, barbs 340 do not extend outwardly past the generally cylindrical stent body. After dilating stent 66 with a balloon, barbs 340 expand outwards with a generally circumferential and radial direction to penetrate into the annulus or mitral valve tissue. A rotation can be provided by deformation of hinges 282 and/or stent struts 118 in the vicinity of barbs 340 that aids in driving barbs 340 into adjacent tissues. FIG. 48B illustrates a corresponding end view.

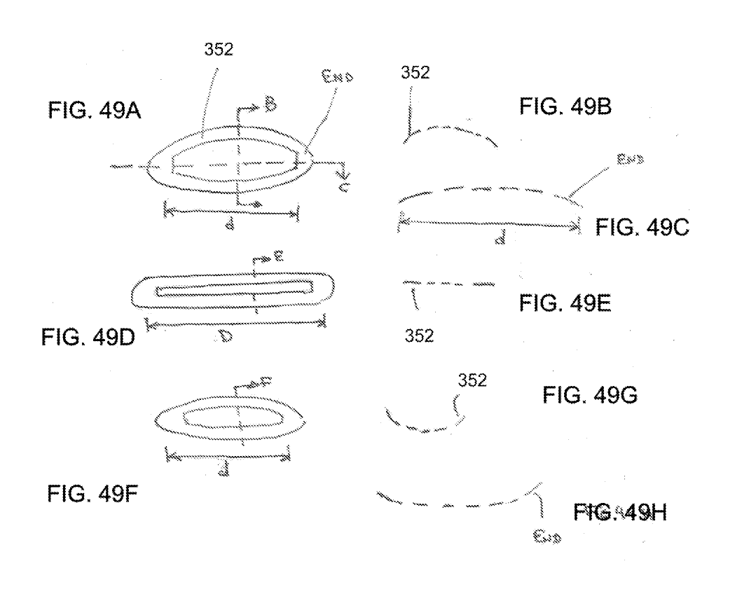

Barbs 340 of the present invention can be formed in part from a bistable geometry such that barb 340 will not extend outwards into adjacent tissue until the pressure applied by the dilation balloon exceeds a specified level such a above 0.5-3 atm (range 0.25-6 atm). As shown in FIGS. 49A-49B a bistable geometry can comprise a generally spherical segment or section formed from two thin-walled strips 352 of metal such as NiTi, SS, or other metal. The ends are separated by distance, d. Upon moving the ends to a larger distance, D, as shown in FIGS. 49D-49E, the bistable metal undergoes shear and expansion strain that store energy in the metal. Continuation of the movement causes the bistable portion 350 of barb 240 to assume a configuration as shown in FIGS. 49F-49H; here the metal bistable portion 350 of barb 340 has shifted its configuration from an upwards bend as shown in FIGS. 49B and 49C to a downwards bend as shown in FIGS. 49G and 49H. This bistable geometry can be used to provide a barb 340 that is contained within the generally tubular stent body during stent expansion after release from sheath 144 and after low-pressure dilation of stent 66 below a specified pressure. Above the specified pressure the bistable barb 340 will flip outwards and extend into the tissue surrounding stent 66 to provide fixation of stent 66 to mitral annulus 20 or native valve leaflets 22.

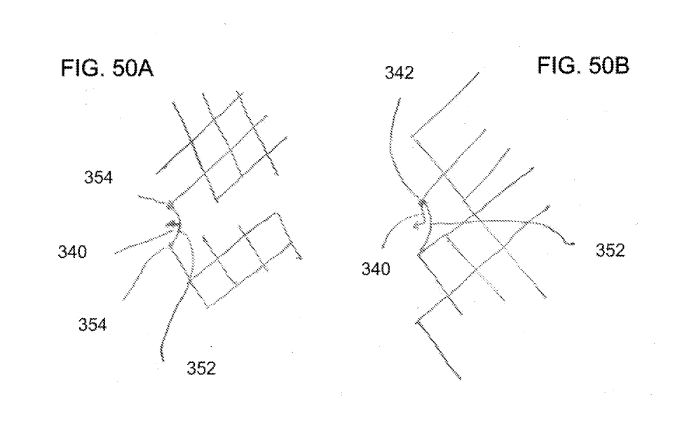

In one embodiment as shown in FIG. 50A a bistable metal strip 352 is located between two fixed points of the stabilizing stent. Each fixed points can be located along a stent strut 118 of stent 66 or it can be located between two struts of the stent. Attached to the metal strip 352 and directed outwards is a metal barb 340. Upon dilation of stent 66 via a dilation balloon above a specified pressure, the bistable metal strip 352 will invert and cause the metal barb 340 to extend outwards beyond the stent body and into adjacent tissue of mitral annulus 20 or native valve leaflets 22.

In an alternate embodiment as shown in FIG. 50B, strip 352 can be attached to the stabilizing stent waist 84 or upper bulb 82 or lower bulb 84 via a BE barb hinge 342. Upon expansion of stent 66 via a dilation balloon, the bistable strip 352 will invert and make contact with barb 340 causing it to pivot outwards and into the tissue as described above. Barbs as described herein can be incorporated into other embodiments of the present invention described above. Structures comprising barbs 340, including embodiments comprising bistable portions 350 and/or bistable strips 352 can be incorporated into SE, BE, and combination BE and SE stabilizing stent configurations described above. The BE stent can be delivered at a balloon dilation pressure that is below that which is required to activate the bistable barbs; once the placement of stent 66 is deemed acceptable, further balloon dilation to activate the bistable barbs is performed.

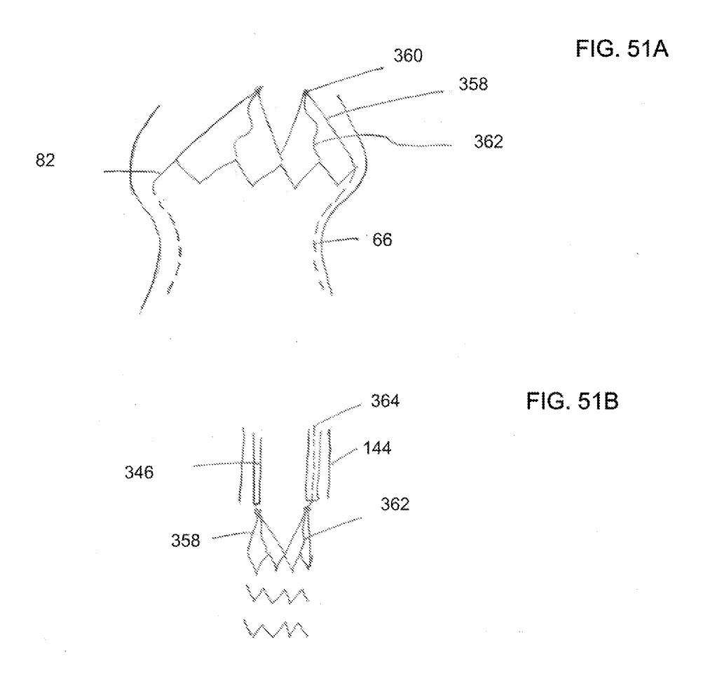

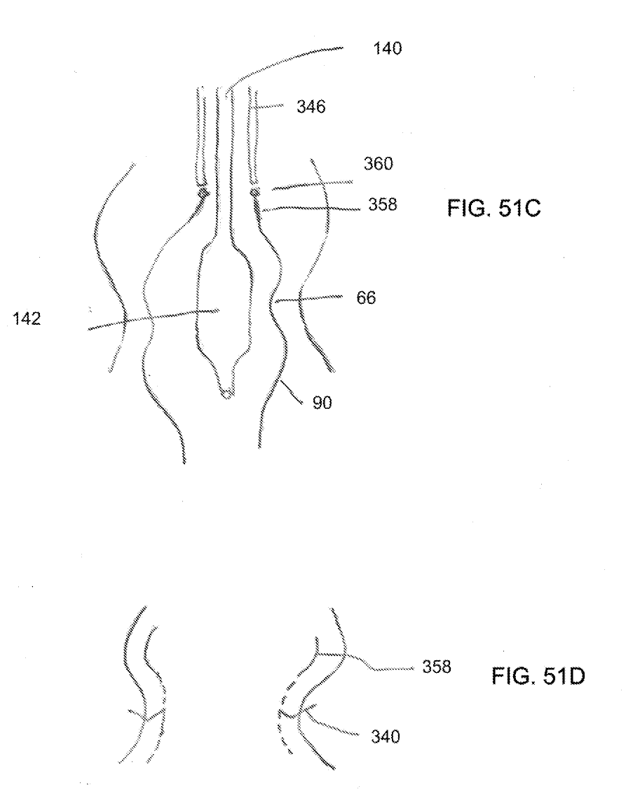

The delivery of stent 66 into the exact location across the mitral annulus 20 is critical to ensure that perivalvular leak does not occur and to ensure that stent migration prevented. The mitral stent-valve and adapters of all of the SE and combination SE and BE embodiments described herein are intended to be repositionable across the annulus after initial release from sheath 144 and be retrievable back into sheath 144 if necessary. To provide the capability of retrieving or repositioning stent 66, the stabilizing stent has recapture elements 356 such as recapture struts 358 or filaments that extend upstream from the stabilizing stent; these recapture struts 358 (see FIG. 51A) can be the upstream struts of the proximal bulb of the stabilizing stent. The recapture struts 358 exert a smaller outward force than the stent struts 118 found in waist 84 of stent 66. The smaller outward force can be obtained by providing thinner walls for the recapture struts 358, providing a lower number of recapture struts 358 than that of waist 84, and/or by providing a longer lever arm or strut length for the recapture struts 358. Attached to the proximal end of recapture struts 358 are attachment sites 360. If a lower number of attachment sites 360 and recapture struts 358 are utilized than found in adjacent stent rings then a recapture connector strut 362 can be included to ensure that the upstream hinge portions of any zig zag stent ring do not prohibit entry of stent 66 back into sheath 144 for retrieval. The recapture connector strut 362 is generally of a thinner dimension and lower strength but serves only to direct the upstream hinge portions of the upstream ring of stent 66 back into sheath 144 for retrieval.

Attachment sites 360 are attached to a release filament 364 that runs along or through the walls of the pusher tube 346 as shown in FIG. 51B. The release filament 364 is releasably attached to attachment sites 360 via one of several mechanisms; for example, release filament 364 can pass through a hole at attachment site 360 and provide a releasable attachment; alternatively the release filament 364 can have be a metal shaft that has a threaded connection to the attachment site 360. Other release mechanisms are anticipated including a clasp mechanism, a thermal release mechanism, an electrical release, etc. Pusher tube 346 is a hollow tube; it serves to hold stent 66 in position while sheath 144 is retracted during release of stent 66 from sheath 144. Pusher tube 346 also serves to allow stent 66 to be repositioned across mitral annulus 20 to ensure that waist 84 of the stent 66 is located adjacent mitral annulus 20.

Once the replacement valve 52 is positioned across mitral annulus 20 a dilation balloon can be passed though pusher tube 346. With the recapture struts 358 being held by pusher tube 346, the balloon can be advanced without concern for movement or migration of stent 66. Dilation of the balloon causes barbs 340 to be pushed outwards into the surrounding tissue of mitral annulus 20 and/or native valve leaflets as shown in FIG. 51D. For stents 66 that are combination BE and SE stents as described above, then the balloon dilation will cause stent 66 to further deform plastically and seat better across mitral annulus 20; such dilation of the BE portions of the combination stent provide the benefits of a BE stent for avoiding continued outward forces against the mitral annulus 20 but provide the benefit of a SE stent by providing immediate blood flow through replacement valve 52 upon release from sheath 144. Following activation of barbs 340 outwards to attach stent 66 to mitral annulus 20 and/or native valve leaflets 22, the attachment sites 360 can be released to fully release stent 66 across mitral annulus 20.

It is understood that the embodiments presented in this application contain features that can be used with other embodiments. For example, the stabilizing stent can have an inner and outer stent, but it can also be comprised of a single waist stent that has a smaller diameter waist region. This stabilizing stent can be used as an adapter, for example, to receive and hold a TAVR device or it can be a one-step implant that has replacement leaflets contained within. BE and SE hinges, open-cell and closed-cell structures, attachment barbs, coverings, and so forth described in one embodiment may be utilized with other embodiments. Other combinations of the described embodiments are also anticipated.

Various modifications can be made to the present invention without departing from the apparent scope thereof.

It is claimed:

1. An adapter for transcatheter implantation of a replacement mitral valve at a mitral annulus, comprising:
a stent with a stabilization portion having a narrowed waist no larger in diameter than the mitral annulus, an enlarged upper bulb significantly larger in diameter than said waist, and an enlarged lower bulb significantly larger in diameter than said waist;
a tract element significantly smaller in diameter than said waist; and,
wherein said tract element is attached to said stabilization portion.

2. The adapter of claim 1, further comprising:
said stent having a stent body;
an attachment element with a plastically deformable hinge deployable by internal dilation;
said attachment element being no larger than said adjacent stent body during delivery; and,
said attachment element extending outward farther than said adjacent stent body to anchor adjacent tissue.

3. The adapter of claim 1, further comprising:
said stent having balloon expandable hinges in series with self expanding hinges;
an expansion limiter adjacent to at least one of said self expanding hinges;
expansion of at least one of said self expanding hinges being limited by said adjacent expansion limiter; and,
the limiting of expansion of at least one of said self expanding hinges by said adjacent expansion limiter forming a narrow waist in said stent when deployed.

4. The adapter of claim 1, further comprising:
said tract element having flexible fabric and axial fibers providing movement of at least a portion of said flexible fabric to function as a temporary valve.

5. The adapter of claim 1, further comprising:
replacement leaflets located in said tract element.

6. The adapter of claim 1, wherein said tract element is located adjacent said waist.

7. The adapter of claim 1, wherein said tract element comprises a tapered portion.

8. Mitral valve replacement apparatus for transcatheter implantation at a mitral annulus, comprising:
a stent with a stabilization portion having a narrowed waist no larger in diameter than the mitral annulus, an enlarged upper bulb significantly larger in diameter than said waist, and an enlarged lower bulb significantly larger in diameter than said waist;
a tract element significantly smaller in diameter than said waist;
wherein said tract element is attached to said stabilization portion;
replacement leaflets located in said tract element; and,
a covering on at least a portion of the stent and the tract element.

9. The mitral valve replacement apparatus of claim 8, further comprising:
said stent having a stent body;
an attachment element with a plastically deformable hinge deployable by internal dilation;
said attachment element being no larger than said adjacent stent body during delivery; and,
said attachment element extending outward farther than said adjacent stent body to anchor adjacent tissue.

10. The mitral valve replacement apparatus of claim 8, further comprising:
said stent having balloon expandable hinges in series with self expanding hinges;
an expansion limiter adjacent to at least one of said self expanding hinges;
expansion of at least one of said self expanding hinges being limited by said adjacent expansion limiter; and,
the limiting of expansion of at least one of said self expanding hinges by said adjacent expansion limiter forming a narrow waist in said stent when deployed.

11. A stent having at least one self expanding hinge that is limited in the extent of expansion by an adjacent expansion limiter.

12. The stent of claim 11, further comprising:
at least one balloon expandable hinge.

13. The stent of claim 11, further comprising:
at least one balloon expandable hinge in series with the at least one self expanding hinge that is limited in the extent of expansion by an adjacent expansion limiter.
